(19) United States
(12) Patent Application Publication
(10) Pub. No.: US 2025/0099598 A1
(43) Pub. Date: Mar. 27, 2025

(54) Analgesic Therapeutics Based on Conjugation of Biocompatible Polymers to Ion Channel Modulators

(71) Applicant: Relevium Medical, Galway (IE)

(72) Inventors: Alison Liddy, Galway (IE); Christophe Labriere, Galway (IE); Mehnaz Ferdousi, Galway (IE); Sahar Avazzadeh, Galway (IE); Simona Cacciapuoti, Galway (IE)

(21) Appl. No.: 18/899,230

(22) Filed: Sep. 27, 2024

Related U.S. Application Data
(63) Continuation-in-part of application No. 18/475,687, filed on Sep. 27, 2023.

(51) Int. Cl. A61K 47/61; A61K 9/00; A61P 19/02; A61P 29/02

(52) U.S. Cl. CPC A61K 47/61; A61K 9/0019; A61P 19/02; A61P 29/02

(57) ABSTRACT

The disclosure is directed towards chemical compositions and methods for treatment of pain, including osteoarthritic pain. The compositions comprise a group of TRPV1 ion channel modulator derivatives, including capsaicinoid derivatives, which are capable of covalently linking to a polymer. The disclosed TRPV1 ion channel modulator derivatives are covalently linked to a polymer and alternatively or additionally said ion channel modulator derivatives are mixed with the polymer. Also disclosed are methods for treatment of pain using the disclosed compounds via injection to a location of the pain or to a vicinity of the location of pain. The disclosed compositions and methods are advantageous because they exhibit sustained release of TRPV1 ion channel modulators, attenuation of initial acute pain associated with TRPV1 modulators, and an ability to be used in high concentrations, thereby resulting in efficacious treatment of pain, including osteoarthritic knee joint pain.

### FIELD OF INVENTION

This invention relates to compositions and methods related to the modulation of excitable tissue activity. In particular these tissues include nerves, and the compositions and methods are specifically for use in conditions including pain or other cases where there is inappropriate nerve activity. The invention also relates to methods of manufacture the compositions.

### BACKGROUND OF INVENTION

Pain has several different classifications including those based on origin, and those based on duration. The origin of pain may for example be nociceptive or neuropathic. Nociceptive pain is a type of pain caused by damage to body tissue, which can be caused by external injury or a condition. Neuropathic pain is caused by damage or dysfunction of the nervous system. The duration of pain may be classified as acute or chronic. Acute pain comes on quickly but lasts a short time (typically less than three months). Chronic pain is persistent and long-lasting.

In all cases the sensation of pain at some level is caused by relaying of electrical signals along different pain sensing neurons to the brain where the signal is interpreted. It follows that suppression of activity of these pain sensing neurons should result in pain relief.

An important condition featuring many different types of pain is osteoarthritis. Osteoarthritis (OA) is an incurable complex disease impacting 350 million people worldwide, including 40 million in Europe. OA is a degenerative joint disease where progressive degradation of bone and cartilage in the affected joint results in severe, typically chronic pain, reduced joint function, and stiffness. Overtime the pain, reduced function, and stiffness gets increasingly worse leading to ever greater patient debilitation.

Osteoarthritis most commonly affects the knee joint, representing 45% of cases (18 million Europeans). Knee OA is the 4th leading cause of disability worldwide, with rapid increasing prevalence due to an ageing population and the growing obesity epidemic. Knee osteoarthritis affects more people globally each year than cancer and heart disease. There is no means to stop disease progression, and no long-lasting effective pain relief treatment. Osteoarthritis pain is particularly severe impacting patients' sleep, mood, and everyday life. Patients with osteoarthritis suffer from chronic pain for an average of 28 years from initial diagnosis, as well as suffering immobility. Inadequate management of chronic pain is directly related to quality of life with one third of people with osteoarthritis over 45 suffering with high levels of depression and sleep deprivation.

Knee osteoarthritis is a painful, debilitating, and progressive disease where no current treatment offers patients effective pain relief without side effects. Current treatment options for knee osteoarthritis patients fall into four categories, with the patient pathway typically progressing through these options with disease progression:
Oral Medications: These include non-steroidal anti-inflammatory drugs (NSAIDs) and opioids. Both these classes of drugs manage the symptoms but are short acting usually requiring daily dosing and have serious side-effect such as organ damage with NSAIDs and dependency with opioids. Furthermore, as no agent stops progression of osteoarthritis, stronger doses are needed as the disease progresses and naturally there is a limit on the safety of increasing the oral doses. Steroid Injectables: Steroids are injected directly into the intra-articular space of the joint to give pain relief. However, the effect lasts for an average of only 2-4 weeks and irreplaceable cartilage is destroyed each time steroids are used leading to limitations on use.
Hyaluronic Acid (HA) Injectables: HA is a major component of the fluid in a normal healthy joint, acting as a lubricant and shock absorber. Commercial HA injectables replace HA in the damaged joint and while safe, provide mild pain relief through a barrier effect. While HA injections are still used by some clinicians, major bodies such as The American Academy of Orthopaedic Surgeons (AAOS) and NICE (UK) no longer recommend them due to insufficient evidence of significant pain relief for osteoarthritis.
Stem Cell and Platelet Injectables: Although currently an area of active research, these products have failed do demonstrate that they are effective in relieving OA pain.
Knee Replacement Surgery: Approximately 5.6% of patients every year opt for knee replacement surgery. This is an end-stage treatment where the damaged knee is replaced with an artificial metal implant. This is an expensive highly invasive procedure, that is often associated with a high failure rate and need for revision. Hence, surgery is considered a last-resort treatment for patients with terminally damaged knee joints and not responding to other treatment options. Of relevance and note are the challenges in managing acute pain post-knee replacement surgery. Knee replacement surgery is associated with severe acute pain after surgery, and a high incidence of chronic pain. Currently a multimodal approach to treating the acute pain involving opioids, NSAIDs, and local anaesthetic nerve blocks is often used. Despite these measures to manage the acute pain, many patients are discharged after surgery with pain that can persist for weeks, months, or years with this chronic pain difficult to treat.

It is noteworthy that the current established treatments do not include an option primarily targeting modulation of afferent pain nerve activity. Suppression of inappropriate pain sensing nerve activity would be of clear benefit in osteoarthritis where there is such pain.

In addition to application in OA conditions (including post-knee replacement surgery), it is recognised that agents that can be used to modulate the activity of pain sensing neurons may also be useful for other painful conditions such as those featuring neuropathic pain and/or inflammatory pain. These conditions include painful bladder syndrome, vaginal atrophy, and many others.

Further, in all cases it would be desirable that a therapy offers immediate, long-lasting, and safe modulation of nerve cell activity, and only in the specific nerve cell type—for example pain sensing neurons.

In addressing the above-mentioned issues, the inventors here focused on pharmacologically active agents selectively targeting pain sensing neurons. In particular, the inventors explored the modulation of ion channels in the membranes of peripheral pain sensing neurons such as the transient receptor potential cation channel subfamily V member 1 (TRPV1) channels and voltage gated sodium (Nav) by ion channel modulators. These channels are critical to the transmission of pain signals to the brain, with suppression of their overactivity leading to pain relief.

The use of ion channel modulators has been reported in certain limited conditions: The classic local anaesthetic class of drugs (e.g., lidocaine) act on the intracellular part of the Nav channel as ion channel modulators. While effective at pain suppression, local anaesthetics typically suffer from low potency, short half-life, poor bioavailability, and lack of selectivity with associated adverse effects. These factors limit their utility in cases where there is a need for sustained pain relief. Hence, these agents have some limited topical uses but are more often given by injection for indications where there is a need for transient pain suppression, for example before surgical procedures. Attempts have been made to prolong their duration of action using technologies such as liposomes extending effects from hours to 2-3 days. However, the inherent low potency of these agents results in the need for large volumes to realise this increase in duration of action. For example, bupivacaine is usually administered at a strength of 0.25-0.75% (i.e., 25 mg-75 mg in a 10 mL volume), effect lasting for approximately 6 hours. The long-acting liposomal product Exparel® has a maximal dosage of 266 mg/20 mL with the SPC often indicating a recommendation to dilute depending on the indication. Exparel® has a duration of action of up to 72 hours. It is noted that intra-articular injection volumes into the knee as an example are of the order of 2-6 mL volume. High injection volumes are prohibitive in that the amount of active therapeutic is limited and high injection volumes may result in further complications. In addition, the ubiquitous presence of Nav channels on many different nerve types and other excitable tissues can lead to side effects. For example, while local anaesthetics preferentially act on small fibre nerves such as those transmitting pain, they can also suppress the activity of larger nerves such as those involved in proprioception and motor function. While administration to the knee joint in the case of knee OA should limit systemic adverse effects a further limitation to use there is the chondrotoxic potential of these agents. Consequently, there is a need for compositions and methods to treat conditions such as OA, that provide longer lasting effects, and that allow administration of higher concentrations of anaesthetics in low volumes.

The TRPV1 channel is found mainly on small to medium fibres—i.e., mainly on pain sensing fibres. Importantly, these channels are upregulated and sensitised in pain conditions, such as OA. These features make them an attractive target for candidate pain relieving drugs. However, attempts to target TRPV1 to date has been met with limited success. Candidate TRPV1 antagonists (a type of ion channel modulator) for example have persistent issues with side effects such as hyperthermia and alteration of heat sensation. TRPV1 agonists such as capsaicin and resiniferatoxin paradoxically show more translation potential. TRPV1 agonists initially activate TRPV1 channels, which leads to a temporary increase in pain perception due to the excitation of pain-sensing neurons. This initial acute pain response is often considered a major obstacle in developing TRPV1 agonists for clinical use, including OA. However, after sustained or high-dose exposure, these agonists lead to long-term desensitization of TRPV1 channels, reducing their sensitivity to painful stimuli. This desensitization holds significant therapeutic potential for managing chronic pain.

Clinical trials involving the injection of 1 mg of capsaicin showed both Injection Site Pain shortly after administration (intense for some patients) and duration of initial pain lasting a few hours to a day. Pain management was achieved via local anaesthetics and cold compresses. In certain cases, pre-administration of lidocaine or other topical anaesthetics can be used to help minimize the injection pain. While promising, the acute pain sensitisation requires significant levels of pre-treatment to control. In addition, the effect may begin to wane towards the end of the dosing interval leading to breakthrough pain before the next application.

Hence, there is a need for new treatments for pain in conditions such as knee OA which are efficacious, safe, and long-lasting. Ion channel modulators (including some neurotoxins) acting on pain sensing neurons hold promise for use in these conditions but have challenges in their current form.

This disclosure provides solutions to the above-mentioned issues of the current art by addressing the issues of how to provide an ion channel modulator in a formula enabling a slower release, allowing administration in higher concentrations and low volumes and minimizing the initial pain.

### SUMMARY OF THE INVENTION

The core concept of this invention is the conjugation and alternatively or additionally mixing of ion channel modulator derivatives to a biocompatible polymer base to overcome the above-described challenges. The conjugation and the optional mixing results in a depot of ion channel modulator which would be released over an extended period of time in vivo. This ensures a controlled release profile that maintains concentrations within the therapeutic range for a prolonged duration, thereby minimizing breakthrough pain and reducing the need for frequent re-administration.

According to one aspect of the present invention, there is provided a composition comprising a polymer in a carrier and
i. At least one ion channel modulator derivative covalently bound to the polymer; and alternatively, or additionally
ii. At least one ion channel modulator derivative, mixed with the polymer.

According to another aspect of the present invention, there is provided methods of localised modulation of nerve cell activity in a subject, the method comprising locally administering an ion channel modulator comprising a polymer in a carrier and
i. At least one ion channel modulator derivative covalently bound to the polymer; and alternatively, or additionally
ii. At least one ion channel modulator derivative, mixed with the polymer.

According to one aspect of the composition for treatment of pain is provided, wherein the composition comprises a TRPV1 ion channel modulator derivative, capable of covalently linking to a polymer, wherein the ion channel modulator derivative is covalently linked to a polymer, and alternatively or additionally the ion channel modulator derivative is mixed with the polymer.

Advantageously, the present invention provides ion channel modulator derivatives, that are covalently bound to the polymer base and are capable of modulating ion flow into nerve cells and to reduce the ability of these cells to generate action potentials. This reduction in action potential translates into long-lasting pain relief when applied to pain sensing neurons (which has been demonstrated both in vitro and in vivo.)

Furthermore, in the case of ion channel modulators of the TRPV1 agonist class when covalently bound to and alternatively or additionally mixed with a polymer base does modulate ion flow into nerve cells and reduce the ability of cells to generate action potentials and reduce the initial excitation phase characteristic of these modulators. This reduction in action potentials with a reduction in initial excitation translates into pain relief with a reduced initial acute period of sensitisation when applied to pain sensing neurons (which is demonstrated both in vitro and in vivo).

The present invention provides a solution, where covalent binding and alternatively or additionally mixing of ion channel modulator derivatives to the polymer offers a long-lasting effect when used for the modulation of nerve cells to provide for pain relief. Moreover, the present invention provides a solution to reduce toxicity, acute pain response, and safety signals related to the use of ion channel modulators.

According to certain embodiments the composition for treatment of pain comprises one or more novel TRPV1 modulator derivatives of TRPV1 agonists selected from the group consisting of (E)-capsaicin, (Z)-capsaicin, dihydrocapsaicin, capsaicin USP, olvanil, palvanil, nonivamide, N-(4-Hydroxy-3-methoxybenzyl)-4-(thiophen-2-yl) butanamide (MSP-3), resiniferatoxin (RTX), tinyatoxin and more generally natural or synthetic capsaicinoids, capsiate, dihydrocapsiate and more generally capsinoids, C18-N-acylethanolamines (e.g., anandamide), vanillotoxin, piperine, zingerone, N-Arachidonoyl Dopamine (NADA), gingerol, shogaol, 6-paradol, evodiamine, scutigeral, cinnamaldehyde, allicin, eugenol and polygodial. Capsaicin USP is defined as a mixture of (E)-capsaicin and dihydrocapsaicin, where dihydrocapsaicin constitutes approximately 30-40% of the total capsaicinoid content.

According to certain embodiments the composition for treatment of pain comprises a TRPV1 ion channel modulator derivative, covalently linked to a polymer, and alternatively or additionally the TRPV1 ion channel modulator derivative mixed with the polymer, wherein the TRPV1 ion channel modulator derivative is a capsaicinoid.

According to certain embodiments the composition for treatment of pain comprises a capsaicin derivative as a TRPV1 ion channel modulator derivative, wherein the capsaicin derivative is characterized by a capsaicinoid modified to allow covalent linking of the capsaicinoid to a polymer via ester bonds, amide bonds, carbamate bonds, carbonate bonds, or cleavable ether bonds with building blocks selected from aliphatic or aromatic dicarboxylic acids preferably succinic acid, adipic acid, or glutaric acid; hydroxy acids preferably lactic acid, glycolic acid, or citric acid; amino-acids peptides preferably beta-alanine, glycine, lysine, or glutamic acid; oligopeptides or polypeptides; diamines preferably 1,6-diaminohexane or ethylene diamine; aminoalcohols preferably serinol or 1-amino-2-propanol; diols preferably ethylene glycol, 1,6-hexanediol, or glycerol; alkyloxycarbonyloxymethyl; and/or N-alkyl-N-alkyloxycarbonylaminomethyl.

According to certain embodiments the composition for treatment of pain comprises capsaicin derivative as a TRPV1 ion channel modulator derivative covalently linked to a polymer and alternatively or additionally an ion channel modulator derivative mixed with the polymer, wherein the TRPV1 ion channel modulator derivative is selected from the group consisting of: (E)-2-methoxy-4-[(8-methylnon-6-enamido)methyl]phenyl glycinate; (E)-2-methoxy-4-[(8-methylnon-6-enamido)methyl]phenyl 3-aminopropanoate; (E)-2-methoxy-4-[(8-methylnon-6-enamido)methyl]phenyl 2-[2-(2 aminoethoxy)ethoxy]acetate; (E)-2-methoxy-4-[(8-methylnon-6-enamido)methyl]phenyl 3-(2-aminoethoxy)propanoate; (E)-2-methoxy-4-[(8-methylnon-6-enamido)methyl]phenyl 2-(2-aminoethoxy)acetate; (E)-2-methoxy-4-[(8-methylnon-6-enamido)methyl]phenyl (5-aminopentyl)carbamate; (E)-2-methoxy-4-[(8-methylnon-6-enamido)methyl]phenyl [2-(2-aminoethoxy)ethyl]carbamate; 2-methoxy-4-{[(E)-8-methylnon-6-enamido]methyl]phenyl trans-4-aminocyclohexane-1-carboxylate; (E)-2-methoxy-4-[(8-methylnon-6-enamido)methyl]phenyl 3-aminopyrrolidine-1-carboxylate; (E)-2-methoxy-4-[(8-methylnon-6-enamido)methyl]phenyl (2-monoethyl)(isopropyl)carbamate; 2-methoxy-4-{[(E)-8-methylnon-6-enamido]methyl}phenyl L-isoleucinate; 2-methoxy-4-{[(E)-8-methylnon-6-enamido]methyl}phenyl trans-4-(2-aminoacetamido)cyclohexane-1-carboxylate; 2-methoxy-4-{[(E)-8-methylnon-6-enamido]methyl}phenyl trans-4-(2-aminopropanamido)cyclohexane-1-carboxylate; (E)-2-methoxy-4-[(8-methylnon-6-enamido)methyl]phenyl (3-aminopropanoyl)-L-prolinate; (E)-2-methoxy-4-[(8-methylnon-6-enamido)methyl]phenyl piperidine-2-carboxylate; (E)-2-methoxy-4-[(8-methylnon-6-enamido)methyl]phenyl glycyl-L-prolinate; (E)-2-methoxy-4-[(8-methylnon-6-enamido)methyl]phenyl 1-glycylpiperidine-2-carboxylate; (E)-2-methoxy-4-[(8-methylnon-6-enamido)methyl]phenyl 1-(3-aminopropanoyl) piperidine-2-carboxylate; (E)-2-methoxy-4-[(8-methylnon-6-enamido)methyl]phenyl 2-[2-(2-aminoethoxy)ethoxy]acetate; (E)-2-methoxy-4-[(8-methylnon-6-enamido)methyl]phenyl L-valinate; (E)-2-methoxy-4-[(8-methylnon-6-enamido)methyl]phenyl (3-aminopropanoyl)-L-valinate; 2-methoxy-4-{[(E)-8-methylnon-6-enamido]methyl}phenyl (1S,2R)-2-aminocyclopentane-1-carboxylate; (E)-2-methoxy-4-[(8-methylnon-6-enamido)methyl]phenyl (3-aminopropanoyl)-D-prolinate; and (E)-2-methoxy-4-[(8-methylnon-6-enamido)methyl]phenyl (3-aminopropanoyl)-DL-prolinate; as their salts or free forms.

According to certain embodiments the composition for treatment of pain comprises capsaicinoid derivative as a TRPV1 ion channel modulator derivative covalently linked to a polymer and alternatively or additionally an ion channel modulator derivative mixed with the polymer, wherein the (E)-capsaicin derivative described in the previous embodiment is replaced by: (Z)-capsaicin.

According to certain embodiments the composition for treatment of pain comprises capsaicinoid derivative as a TRPV1 ion channel modulator derivative covalently linked to a polymer and alternatively or additionally an ion channel modulator derivative mixed with the polymer, wherein the (E)-capsaicin derivative described in the previous embodiment is replaced by: dihydrocapsaicin.

According to certain embodiments the composition for treatment of pain comprises capsaicinoid derivative as a TRPV1 ion channel modulator derivative covalently linked to a polymer and alternatively or additionally an ion channel modulator derivative mixed with the polymer, wherein the (E)-capsaicin derivative described in the previous embodiment is replaced by: capsaicin USP.

According to certain embodiments the composition for treatment of pain comprises capsaicinoid derivative as a TRPV1 ion channel modulator derivative covalently linked to a polymer and alternatively or additionally an ion channel modulator derivative mixed with the polymer, wherein the (E)-capsaicin derivative described in the previous embodiment is replaced by: resiniferatoxin.

According to certain embodiments the composition for treatment of pain comprises capsaicinoid derivative as a TRPV1 ion channel modulator derivative covalently linked to a polymer and alternatively or additionally an ion channel modulator derivative mixed with the polymer, wherein the (E)-capsaicin derivative described in the previous embodiment is replaced by: tinyatoxin.

According to certain aspects the composition for treatment of pain comprises one or more of (E)-2-methoxy-4-[(8-methylnon-6-enamido)methyl]phenyl glycinate-introduced sodium hyaluronate; (E)-2-methoxy-4-[(8-methylnon-6-enamido)methyl]phenyl 3-aminopropanoate-introduced sodium hyaluronate; (E)-2-methoxy-4-[(8-methylnon-6-enamido)methyl]phenyl 3-aminopropanoate-introduced sodium hyaluronate; (E)-2-methoxy-4-[(8-methylnon-6-enamido)methyl]phenyl 3-(2-aminoethoxy)propanoate-introduced sodium hyaluronate; (E)-2-methoxy-4-[(8-methylnon-6-enamido)methyl]phenyl 3-aminopropanoate-introduced sodium hyaluronate; (E)-2-methoxy-4-[(8-methylnon-6-enamido)methyl]phenyl 3-aminopropanoate-introduced sodium hyaluronate; (E)-2-methoxy-4-[(8-methylnon-6-enamido)methyl]phenyl 3-(2-aminoethoxy)propanoate-introduced sodium hyaluronate; (E)-2-methoxy-4-[(8-methylnon-6-enamido)methyl]phenyl 3-aminopropanoate-introduced sodium hyaluronate; (E)-2-methoxy-4-[(8-methylnon-6-enamido)methyl]phenyl 3-(2-aminoethoxy)propanoate-introduced sodium hyaluronate; (E)-2-methoxy-4-[(8-methylnon-6-enamido)methyl]phenyl 3-(2-aminoethoxy)propanoate-introduced sodium hyaluronate; (E)-2-methoxy-4-[(8-methylnon-6-enamido)methyl]phenyl 3-(2-aminoethoxy)propanoate-introduced sodium hyaluronate; (E)-2-methoxy-4-[(8-methylnon-6-enamido)methyl]phenyl [2-(2-aminoethoxy)ethyl]carbamate introduced sodium hyaluronate; (E)-2-methoxy-4-[(8-methylnon-6-enamido)methyl]phenyl (5-aminopentyl)carbamate-introduced sodium hyaluronate; (E)-2-methoxy-4-[(8-methylnon-6-enamido)methyl]phenyl 3-aminopyrrolidine-1-carboxylate-introduced sodium hyaluronate; 2-methoxy-4-{[(E)-8-methylnon-6-enamido]methyl]phenyl trans-4-aminocyclohexane-1-carboxylate-introduced sodium hyaluronate; 2-methoxy-4-{[(E)-8-methylnon-6-enamido]methyl}phenyl L-isoleucinate-introduced sodium hyaluronate; (E)-2-methoxy-4-[(8-methylnon-6-enamido)methyl]phenyl (2-aminoethyl)(isopropyl)carbamate-introduced sodium hyaluronate; 2-methoxy-4-{[(E)-8-methylnon-6-enamido]methyl}phenyl trans-4-(2-aminoacetamido)cyclohexane-1-carboxylate-introduced sodium hyaluronate; 2-methoxy-4-{[(E)-8-methylnon-6-enamido]methyl}phenyl trans-4-(2-aminopropanamido)cyclohexane-1-carboxylate-introduced sodium hyaluronate; (E)-2-methoxy-4-[(8-methylnon-6-enamido)methyl]phenyl piperidine-2-carboxylate-introduced sodium hyaluronate; (E)-2-methoxy-4-[(8-methylnon-6-enamido)methyl]phenyl (3-aminopropanoyl)-L-prolinate-introduced sodium hyaluronate; (E)-2-methoxy-4-[(8-methylnon-6-enamido)methyl]phenyl glycyl-L-prolinate-introduced sodium hyaluronate; (E)-2-methoxy-4-[(8-methylnon-6-enamido)methyl]phenyl 1-glycylpiperidine-2-carboxylate-introduced sodium hyaluronate; (E)-2-methoxy-4-[(8-methylnon-6-enamido)methyl]phenyl 1-(3 aminopropanoyl) piperidine-2-carboxylate-introduced sodium hyaluronate; (E)-2-methoxy-4-[(8-methylnon-6-enamido)methyl]phenyl 2-[2-(2-aminoethoxy)ethoxy]acetate-introduced sodium hyaluronate; (E)-2-methoxy-4-[(8-methylnon-6-enamido)methyl]phenyl glycinate-introduced sodium hyaluronate; (E)-2-methoxy-4-[(8-methylnon-6-enamido)methyl]phenyl L-valinate-introduced sodium hyaluronate; (E)-2-methoxy-4-[(8-methylnon-6-enamido)methyl]phenyl (3-aminopropanoyl)-L-valinate-introduced sodium hyaluronate; 2-methoxy-4-{[(E)-8-methylnon-6-enamido]methyl}phenyl (1S,2R)-2-aminocyclopentane-1-carboxylate-introduced sodium hyaluronate; (E)-2-methoxy-4-[(8-methylnon-6-enamido)methyl]phenyl (3-aminopropanoyl)-D-prolinate-introduced sodium hyaluronate; and (E)-2-methoxy-4-[(8-methylnon-6-enamido)methyl]phenyl (3-aminopropanoyl)-DL-prolinate-introduced sodium hyaluronate.

According to certain aspects the composition for treatment of pain comprises one or more capsaicinoid derivative-introduced sodium hyaluronate wherein the (E)-capsaicin derivative described in the previous embodiment is replaced by: (Z)-capsaicin.

According to certain aspects the composition for treatment of pain comprises one or more capsaicinoid derivative-introduced sodium hyaluronate wherein the (E)-capsaicin derivative described in the previous embodiment is replaced by: dihydrocapsaicin.

According to certain aspects the composition for treatment of pain comprises one or more capsaicinoid derivative-introduced sodium hyaluronate wherein the (E)-capsaicin derivative described in the previous embodiment is replaced by: capsaicin USP.

According to certain aspects the composition for treatment of pain comprises one or more capsaicinoid derivative-introduced sodium hyaluronate wherein the (E)-capsaicin derivative described in the previous embodiment is replaced by: resiniferatoxin.

According to certain aspects the composition for treatment of pain comprises one or more capsaicinoid derivative-introduced sodium hyaluronate wherein the (E)-capsaicin derivative described in the previous embodiment is replaced by: tinyatoxin.

(E)-capsaicin, (Z)-capsaicin, dihydrocapsaicin, RTX and tinyatoxin are represented by the following formulas, capsaicin USP being by a mixture of (E)-capsaicin and dihydrocapsaicin, where dihydrocapsaicin constitutes approximately 30-40% of the total capsaicinoid content.

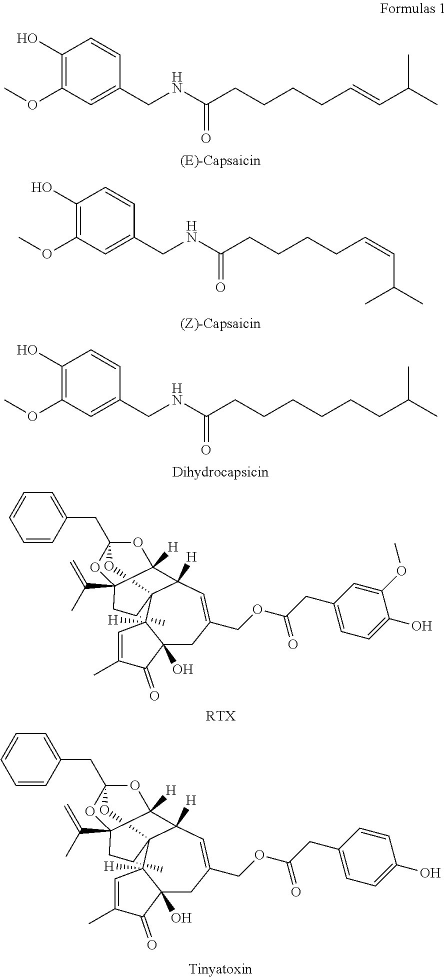

The introduced sodium hyaluronate compounds of the invention are represented by the following formulas:

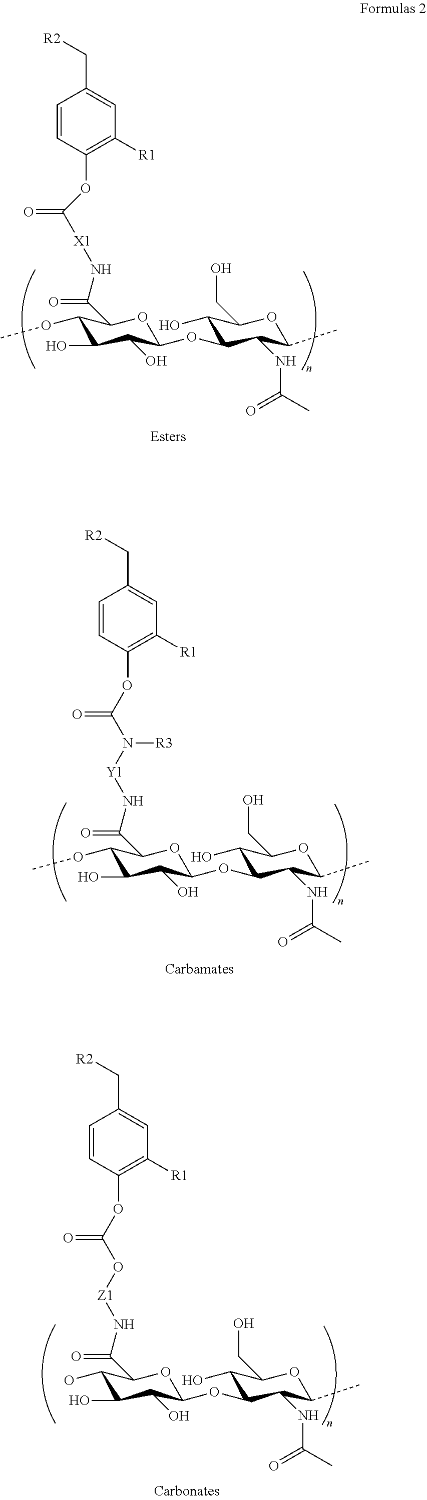

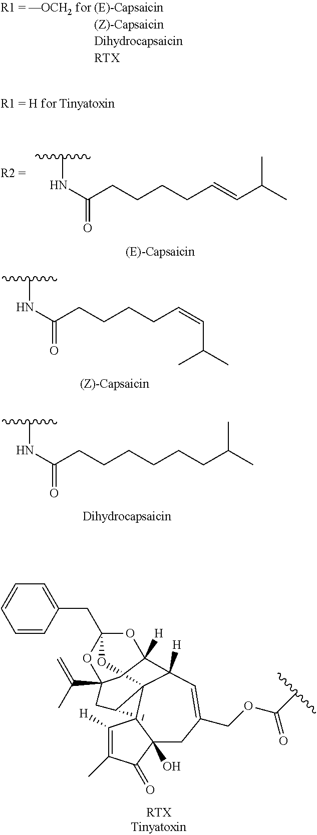

where —NH—X1-CO—, —NH—Y1-NR3-, —NH—Z1-O— are chosen among aliphatic or aromatic dicarboxylic acids preferably succinic acid, adipic acid, or glutaric acid; hydroxy acids preferably lactic acid, glycolic acid, or citric acid; amino-acids peptides preferably beta-alanine, glycine, lysine, or glutamic acid; oligopeptides or polypeptides; diamines preferably 1,6-diaminohexane or ethylene diamine; aminoalcohols preferably serinol or 1-amino-2-propanol; diols preferably ethylene glycol, 1,6-hexanediol, or glycerol; and where R3 is an alkyl or aryl group including optionally heteroatoms.

The introduced sodium hyaluronate compounds of the invention are also represented by the following formulas:

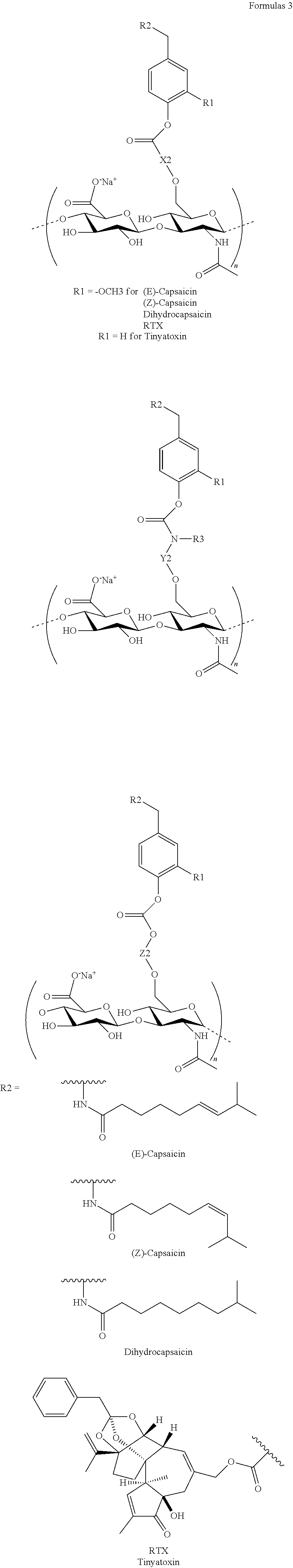

where —NH—X2-CO—, —NH—Y2-NR3-, —NH—Z2-O— are chosen among aliphatic or aromatic dicarboxylic acids preferably succinic acid, adipic acid, or glutaric acid; hydroxy acids preferably lactic acid, glycolic acid, or citric acid; amino-acids peptides preferably beta-alanine, glycine, lysine, or glutamic acid; oligopeptides or polypeptides; diamines preferably 1,6-diaminohexane or ethylene diamine; aminoalcohols preferably serinol or 1-amino-2-propanol; diols preferably ethylene glycol, 1,6-hexanediol, or glycerol; and where R3 is an alkyl or aryl group including optionally heteroatoms.

According to certain embodiments the composition for treatment of pain comprises an ion channel modulator derivative, preferably a TRPV1 modulator derivative and more preferably a capsaicin derivative covalently linked to a polymer, and alternatively or additionally said ion channel modulator derivative is mixed with the polymer, wherein the polymer is a biocompatible polymer and is selected from the group consisting of chitosan, alginic acid and alginate, cellulose derivatives (e.g., methylcellulose, hydroxypropyl methylcellulose), hyaluronic acid (HA) and its derivatives, sodium hyaluronate and its derivatives, starch and modified starch polymers, chondroitin sulfate, xanthan gum, fucoidan, dextran, carrageenan, pectin and more generally polysaccharides; polyethylene glycol (PEG), poly(lactic-co-glycolic acid) (PLGA), polylactic acid (PLA), polyglycolide (PGA), polycaprolactone (PCL), polyanhydrides, polyvinyl alcohol (PVA), poly(hydroxybutyrate) (PHB), poly(ester-amides), poly(ethylene glycol) diacrylate (PEGDA), dendrimer, gelatin, poly(aspartic acid), polyglutamic acid (PGA), poly(glycerol sebacate) (PGS), silk fibroin, fibrin, poly(ethylene-co-vinyl acetate) (PEVA), poly(L-lysine), poly(2-oxazoline), poly(β-amino esters), polyhydroxyalkanoates (PHA); or combinations thereof. Preferably the polymer is selected from the group consisting of hyaluronic acid (HA); derivatives of hyaluronic acid (HA); sodium hyaluronate; derivatives of sodium hyaluronate; and any combination thereof. Preferably the molecular weight of the polymer is between 50 and 3,000 kDa, more preferably between 500 and 3000 kDa The polymer may be cross linked.

According to certain embodiments, the composition for treatment of pain comprises a TRPV1 modulator derivative covalently linked to a polymer, and alternatively or additionally mixed with the polymer, and the ion channel modulator is a capsaicinoid derivatized before being covalently linked to the polymer. The covalent links between the polymer and the capsaicin derivative may be selected from the group consisting of ester bonds, amide bonds, carbamate bonds, carbonate bonds, and cleavable ether bonds. The derivatization of the ion channel modulator may be achieved by coupling a building block from the group of aliphatic or aromatic dicarboxylic acids (such as succinic acid, adipic acid, and glutaric acid), hydroxy acids (such as lactic acid, glycolic acid, and citric acid), amino-acids, peptides (such as beta-alanine, glycine, lysine, or glutamic acid), oligopeptides, polypeptides, diamines (such as 1,6-diaminohexane or ethylene diamine), aminoalcohols (such as serinol or 1-amino-2-propanol), diols (such as ethylene glycol, 1,6-hexanediol, or glycerol), alkyloxycarbonyloxymethyl or N-alkyl-N-alkyloxycarbonylaminomethyl. The amino acids and peptides may be incorporated in their natural configuration, unnatural configuration, or maybe be incorporated as racemic mixtures.

According to certain embodiments, the composition for the treatment of pain comprises a TRPV1 modulator derivative covalently linked to a polymer, and alternatively or additionally said ion channel modulator derivative mixed with the polymer, wherein the polymer is covalently linked to the ion channel modulator derivative in a mass:mass ratio in a range of 1:0.001 to 1:0.5 polymer to ion channel modulator derivative, more preferably between 1:0.01 to 1:5, and even more preferably between 1:0.01 to 1:0.3.

It is an object of the invention to provide a composition for pain relief comprising a carrier; a polymer selected from the group consisting of hyaluronic acid (HA); derivatives of hyaluronic acid (HA); sodium hyaluronate; derivatives of sodium hyaluronate; and any combination thereof; at least one TRPV1 modulator, preferably a capsaicin derivative covalently linked to the polymer from 1% to 50% w/w ratio, preferably 10-15% w/w ratio, and the composition has a sustained release of the ion channel modulator. Preferably the ion channel modulator release takes 0-12 months. The composition is suitable for administration as an injection.

It is an object of this invention to provide a method to release at least one ion channel modulator in a sustained and prolonged manner at a location of pain, by injecting a composition for treatment of pain, wherein the composition comprises an ion channel modulator derivative, covalently linked to a polymer, and alternatively or additionally said ion channel modulator derivative mixed with the polymer, wherein said ion channel modulator derivative is a TRPV1 channel modulator derivative, preferably a capsaicinoid derivative, injected at the site of pain or into a vicinity of the location of the pain. The injection volume may be 1-10 mL, preferably 1-5 mL, and most preferably 2-4 mL, such as 3 mL. One injection may comprise from 1 μg to 100 mg of ion channel modulator derivative which preferably is a capsaicinoid derivative.

### The Polymer

The polymer may be natural or synthetic. The polymer may be biocompatible. In a preferred embodiment, the polymer is a biodegradable polymer. Preferable polymers are non-toxic to mammalian subjects at therapeutically acceptable levels.

In one embodiment, the polymer is selected from one or more of chitosan, alginic acid and alginate, cellulose derivatives (e.g., methylcellulose, hydroxypropyl methylcellulose), hyaluronic acid (HA) and its derivatives, sodium hyaluronate and its derivatives, starch and modified starch polymers, chondroitin sulfate, xanthan gum, fucoidan, dextran, carrageenan, pectin and more generally polysaccharides; polyethylene glycol (PEG), poly(lactic-co-glycolic acid) (PLGA), polylactic acid (PLA), polyglycolide (PGA), polycaprolactone (PCL), polyanhydrides, polyvinyl alcohol (PVA), poly(hydroxybutyrate) (PHB), poly(ester-amides), poly(ethylene glycol) diacrylate (PEGDA), dendrimer, gelatin, poly(aspartic acid), polyglutamic acid (PGA), poly(glycerol sebacate) (PGS), silk fibroin, fibrin, poly(ethylene-co-vinyl acetate) (PEVA), poly(L-lysine), poly(2-oxazoline), poly(β-amino esters), polyhydroxyalkanoates (PHA); or combinations thereof.

The polymer may comprise or consist of glycosaminoglycans. In one embodiment, the polymer comprises, or consists of, hyaluronic acid (HA) or sodium hyaluronate. Hyaluronic acid (HA) may also be known as hyaluronan.

In another embodiment the polymer is a derivative of HA, such as a cross-linked HA, or HA with functional groups designed to favourably adjust the physicochemical properties or facilitate drug loading.

In another embodiment, the polymer may comprise or consist of a carbohydrate polymer such as dextrans, cyclodextrins, xanthan, alginate, gellan, pullulan, mannan, chitin, chitosan, agarose, or guar.

The polymer may have a molecular weight sufficient to form a gel under normal environmental conditions, for example at ambient room temperature (i.e., about 24° C.). In another embodiment, the polymer may have a molecular weight sufficient to form a solution or a colloid with a carrier (for example an aqueous carrier) under normal environmental conditions, for example at room temperature (i.e., about 24° C.). The polymer may be at a sufficient concentration and have a sufficient molecular weight to form a solution or a colloid or a gel with the carrier, under normal environmental conditions for example at room temperature (i.e., about 24° C.).

The polymer may have a molecular weight of at least 5 kDa and up to 3000 kDa. In one embodiment, the polymer has a molecular weight of between about 5 kDa and about 3000 kDa. In another embodiment, the polymer may have a molecular weight of between about 10 kDa and about 3000 kDa. In one embodiment, the polymer has a molecular weight of between about 20 kDa and about 3000 kDa. In certain embodiments, the polymer may have a molecular weight of at least 50 kDa and up to 3000 kDa. In a preferred embodiment, the polymer may have a narrower range between about 50 kDa and about 2500 kDa, or between about 50 kDa and about 2400 kDa. In an alternative embodiment, the polymer may have a molecular weight between about 50 kDa and about 2200 kDa. In other embodiments, the polymer may have a narrower molecular weight range between about 500 kDa and about 2400 kDa. In an alternative embodiment, the polymer may exhibit a molecular weight between 2200 kDa and 2400 kDa.

In an embodiment where the polymer is hyaluronic acid or sodium hyaluronate (HA), HA may have a molecular weight of at least 5 kDa and up to 3000 kDa. In one embodiment, HA has a molecular weight of between about 5 kDa and about 3000 kDa. In another embodiment, HA may have a molecular weight of between about 10 kDa and about 3000 kDa. In one embodiment, HA has a molecular weight of between about 20 kDa and about 3000 kDa. In certain embodiments, HA may have a molecular weight of at least 50 kDa and up to 3000 kDa. In a preferred embodiment, HA may have a narrower range between about 50 kDa and about 2500 kDa, or between about 50 kDa and about 2400 kDa. In an alternative embodiment, HA may have a molecular weight between about 50 kDa and about 2200 kDa. In other embodiments, HA may have a narrower molecular weight range between about 500 kDa and about 2400 kDa. In an alternative embodiment, HA may exhibit a molecular weight between 2200 kDa and 2400 kDa.

The polymer may be cross-linked, for example to form a gel, or in another embodiment, the polymer may not require cross-linking. The polymer cross-linking may be covalent cross-linking or ionic or a blend of both, for example via charged functional moieties bound to the polymer.

The polymer, such as hyaluronic acid, may be provided in a concentration of up to 30% w/v in the carrier. In another embodiment, the polymer, such as hyaluronic acid, may be provided in a concentration of between 5 mg/mL and 20 mg/mL.

### Derivatization and Conjugation to the Polymer

In one embodiment the ion channel modulator derivative(s) are directly covalently linked to the polymer, such as HA. In one embodiment, the ion channel modulator is derivatized before being covalently linked to the polymer, such as HA. The covalent links between the polymer and the ion channel modulator derivative may be selected from the group consisting of ester bonds, amide bonds, carbamate bonds, carbonate bonds, and cleavable ether bonds.

The bonds may exhibit varying stability under different environmental conditions. By incorporating chemically labile bonds, bioresponsive materials can be engineered and they can be disassembled upon certain conditions.

The derivatization of the ion channel modulator may be achieved by coupling a building block from the group of aliphatic or aromatic dicarboxylic acids (such as succinic acid, adipic acid, and glutaric acid), hydroxy acids (such as lactic acid, glycolic acid, and citric acid), amino-acids, peptides (such as beta-alanine, glycine, lysine, or glutamic acid), oligopeptides, polypeptides, diamines (such as 1,6-diaminohexane or ethylene diamine), aminoalcohols (such as serinol or 1-amino-2-propanol), diols (such as ethylene glycol, 1,6-hexanediol, or glycerol), alkyloxycarbonyloxymethyl or N-alkyl-N-alkyloxycarbonylaminomethyl. The amino acids and peptides may be incorporated in their natural configuration, unnatural configuration, or maybe be incorporated as racemic mixtures.

A key advantage of covalently binding or optionally mixing the ion channel modulator derivative(s) with the polymer is its ability to remain in situ, such as within a joint, for extended periods, thereby providing long-lasting effects like pain relief. Without polymer conjugation, the ion channel modulator derivative is more rapidly cleared from the joint. By contrast, the covalent binding or mixing with the polymer can result in a therapy that supports a clinically acceptable injection frequency, such as 2-4 times per year. Conjugation to the polymer may also reduce degradation and limit the escape of the ion channel modulator(s) through trans-synovial flux. Additionally, a chemically modified HA polymer backbone may be further protected from hyaluronidase degradation and mechanical wear caused by joint compression. The specific distribution of the ion channel modulator(s) can avoid non-targeted biodistribution. Furthermore, the ion channel modulator derivative(s) conjugated to the polymer may provide the benefit of charge maintenance, preventing the loss of electrostatic charge within the joint.

Furthermore, higher doses of ion channel modulator(s) may pose safety concerns as the dose approaches or exceeds the usual therapeutic window, or when the ion channel modulator(s) operates within a very narrow therapeutic window. Advantageously, generating a conjugate by covalently attaching the ion channel modulator(s) or its derivatives to a polymer can provide a safer therapy, as the toxic properties of the ion channel modulator are removed through covalent binding and conjugation.

### The Ion-Channel Modulator

The ion-channel modulator is understood to mean a molecule which interacts pharmacologically with ion-channel(s) on excitable tissues, in particular on nerves.

In a preferred embodiment, the ion channel modulator may target transient receptor potential cation channel subfamily V (TRPV) channels. In one embodiment the ion-channel modulator is a non-specific TRPV antagonist. In another embodiment the ion-channel modulator is a specific TRPV antagonist of one (or more) of the recognised members of the TRPV channel family, such as TRPV1 channels. In another embodiment the ion-channel modulator is an antagonist for specific members of the TRPV1 channel family but with varying affinities. In another embodiment the ion-channel modulator is a mixed agonist/antagonist for the TRPV1 channel (non-specific, specific, or mixed affinities). In another embodiment the ion-channel modulator is an agonist for the TRPV1 channel (non-specific, specific, or mixed). In one embodiment the ion-channel modulator interacts with the TRPV1 channel with reversible binding. In another embodiment the ion-channel modulator interacts with the TRPV1 channel with irreversible binding. In one embodiment the ion-channel modulator interacts with the TRPV1 channel through an extracellular domain. In another embodiment the ion-channel modulator interacts with the TRPV1 channel through an intracellular domain.

In one embodiment, the ion channel modulator is a TRPV1 agonist selected from (E)-capsaicin, (Z)-capsaicin, dihydrocapsaicin, capsaicin USP, olvanil, palvanil, nonivamide, N-(4-Hydroxy-3-methoxybenzyl)-4-(thiophen-2-yl) butanamide (MSP-3), resiniferatoxin (RTX), tinyatoxin and more generally a natural or synthetic capsaicinoid, capsiate, dihydrocapsiate and more generally a capsinoid. In further embodiments the TRPV1 agonist is selected from C18-N-acylethanolamines (e.g., anandamide), vanillotoxin, piperine, zingerone, N-Arachidonoyl Dopamine (NADA), gingerol, shogaol, 6-paradol, evodiamine, scutigeral, cinnamaldehyde, allicin, eugenol and polygodial. In a preferred embodiment, the TRPV1 agonist is selected from (E)-capsaicin, (Z)-capsaicin, dihydrocapsaicin, capsaicin USP, resiniferatoxin (RTX) and tinyatoxin.

According to certain embodiments, the ion channel modulator (E)-capsaicin is derivatized to form an ester bond, amide bond, carbamate bond, carbonate bond, or cleavable ether bond with a building block selected from the group consisting of aliphatic or aromatic dicarboxylic acids (such as succinic acid, adipic acid, and glutaric acid), hydroxy acids (such as lactic acid, glycolic acid, and citric acid), amino-acids, peptides (such as beta-alanine, glycine, lysine, or glutamic acid), oligopeptides, polypeptides, diamines (such as 1,6-diaminohexane or ethylene diamine), aminoalcohols (such as serinol or 1-amino-2-propanol), diols (such as ethylene glycol, 1,6-hexanediol, or glycerol), alkyloxycarbonyloxymethyl or N-alkyl-N-alkyloxycarbonylaminomethyl. The amino-acids and peptides may be incorporated in their natural configuration, unnatural configuration, or maybe be incorporated as racemic mixtures.

According to certain embodiments, the ion channel modulator (Z)-capsaicin is derivatized to form an ester bond, amide bond, carbamate bond, carbonate bond, or cleavable ether bond with a building block selected from the group consisting of aliphatic or aromatic dicarboxylic acids (such as succinic acid, adipic acid, and glutaric acid), hydroxy acids (such as lactic acid, glycolic acid, and citric acid), amino-acids, peptides (such as beta-alanine, glycine, lysine, or glutamic acid), oligopeptides, polypeptides, diamines (such as 1,6-diaminohexane or ethylene diamine), aminoalcohols (such as serinol or 1-amino-2-propanol), diols (such as ethylene glycol, 1,6-hexanediol, or glycerol), alkyloxycarbonyloxymethyl or N-alkyl-N-alkyloxycarbonylaminomethyl. The amino-acids and peptides may be incorporated in their natural configuration, unnatural configuration, or maybe be incorporated as racemic mixtures.

According to certain embodiments, the ion channel modulator dihydrocapsaicin is derivatized to form an ester bond, amide bond, carbamate bond, carbonate bond, or cleavable ether bond with a building block selected from the group consisting of aliphatic or aromatic dicarboxylic acids (such as succinic acid, adipic acid, and glutaric acid), hydroxy acids (such as lactic acid, glycolic acid, and citric acid), amino-acids, peptides (such as beta-alanine, glycine, lysine, or glutamic acid), oligopeptides, polypeptides, diamines (such as 1,6-diaminohexane or ethylene diamine), aminoalcohols (such as serinol or 1-amino-2-propanol), diols (such as ethylene glycol, 1,6-hexanediol, or glycerol), alkyloxycarbonyloxymethyl or N-alkyl-N-alkyloxycarbonylaminomethyl. The amino-acids and peptides may be incorporated in their natural configuration, unnatural configuration, or maybe be incorporated as racemic mixtures.

According to certain embodiments, the ion channel modulator capsaicin USP is derivatized to form an ester bond, amide bond, carbamate bond, carbonate bond, or cleavable ether bond with a building block selected from the group consisting of aliphatic or aromatic dicarboxylic acids (such as succinic acid, adipic acid, and glutaric acid), hydroxy acids (such as lactic acid, glycolic acid, and citric acid), amino-acids, peptides (such as beta-alanine, glycine, lysine, or glutamic acid), oligopeptides, polypeptides, diamines (such as 1,6-diaminohexane or ethylene diamine), aminoalcohols (such as serinol or 1-amino-2-propanol), diols (such as ethylene glycol, 1,6-hexanediol, or glycerol), alkyloxycarbonyloxymethyl or N-alkyl-N-alkyloxycarbonylaminomethyl. The amino-acids and peptides may be incorporated in their natural configuration, unnatural configuration, or maybe be incorporated as racemic mixtures.

According to certain embodiments, the ion channel modulator resiniferatoxin is derivatized to form an ester bond, amide bond, carbamate bond, carbonate bond, or cleavable ether bond with a building block selected from the group consisting of aliphatic or aromatic dicarboxylic acids (such as succinic acid, adipic acid, and glutaric acid), hydroxy acids (such as lactic acid, glycolic acid, and citric acid), amino-acids, peptides (such as beta-alanine, glycine, lysine, or glutamic acid), oligopeptides, polypeptides, diamines (such as 1,6-diaminohexane or ethylene diamine), aminoalcohols (such as serinol or 1-amino-2-propanol), diols (such as ethylene glycol, 1,6-hexanediol, or glycerol), alkyloxycarbonyloxymethyl or N-alkyl-N-alkyloxycarbonylaminomethyl. The amino-acids and peptides may be incorporated in their natural configuration, unnatural configuration, or maybe be incorporated as racemic mixtures.

According to certain embodiments, the ion channel modulator tinyatoxin is derivatized to form an ester bond, amide bond, carbamate bond, carbonate bond, or cleavable ether bond with a building block selected from the group consisting of aliphatic or aromatic dicarboxylic acids (such as succinic acid, adipic acid, and glutaric acid), hydroxy acids (such as lactic acid, glycolic acid, and citric acid), amino-acids, peptides (such as beta-alanine, glycine, lysine, or glutamic acid), oligopeptides, polypeptides, diamines (such as 1,6-diaminohexane or ethylene diamine), aminoalcohols (such as serinol or 1-amino-2-propanol), diols (such as ethylene glycol, 1,6-hexanediol, or glycerol), alkyloxycarbonyloxymethyl or N-alkyl-N-alkyloxycarbonylaminomethyl. The amino-acids and peptides may be incorporated in their natural configuration, unnatural configuration, or maybe be incorporated as racemic mixtures.

Alternatively, the composition may comprise a range of different ion channel modulators, each targeting different ion channels and/or with different patterns of binding. In addition, the proportion of different ion channel modulators can vary. In one embodiment, the TRPV channel modulators comprise the majority.

In one embodiment, the composition may comprise a polymer of a single molecular weight. In another embodiment, the polymer used may have a range of molecular weights. In a further embodiment, a blend of polymers is used, either of the same molecular weight or across a range of molecular weights. Additionally, in another embodiment, a blend of conjugated polymers (where an ion channel modulator derivative is attached) and pristine polymers (unmodified polymers) may be used, either with the same molecular weight or across a range of molecular weights.

In one embodiment, the composition contains a polymer component with a molecular weight between 500 kDa and 3,000 kDa. In a further embodiment, the polymer component has a molecular weight ranging from 500 kDa up to a maximum molecular weight of approximately 10 MDa. In another embodiment, the polymer component has a molecular weight between 2.2 and 2.4 MDa. In one embodiment, the polymer is hyaluronic acid or sodium hyaluronate (HA). In another embodiment, the polymer is a different biocompatible polymer. In another embodiment, the polymer is a blend of different biocompatible polymers.

In one embodiment, the composition comprises or consists of hyaluronic acid or sodium hyaluronate, covalently linked to (E)-capsaicin or a derivative, (Z)-capsaicin or a derivative, dihydrocapsaicin or a derivative, capsaicin USP or a derivative, resiniferatoxin or a derivative, tinyatoxin or a derivative, or combinations thereof. In another embodiment, the composition comprises (E)-capsaicin or a derivative, (Z)-capsaicin or a derivative, dihydrocapsaicin or a derivative, capsaicin USP or a derivative, resiniferatoxin or a derivative, tinyatoxin or a derivative, or combinations thereof, covalently linked and optionally additionally mixed with hyaluronic acid or sodium hyaluronate. In one embodiment, the composition comprises hyaluronic acid or sodium hyaluronate, non-covalently bound and mixed with (E)-capsaicin or a derivative, (Z)-capsaicin or a derivative, dihydrocapsaicin or a derivative, capsaicin USP or a derivative, resiniferatoxin or a derivative, tinyatoxin or a derivative, or combinations thereof.

In one embodiment, the polymer is covalently linked to the ion channel modulator derivative in a mass:mass ratio of about 1:0.2 polymer to ion channel modulator derivative. In another embodiment, the polymer is covalently linked to the ion channel modulator derivative in a mass:mass ratio of about 1:0.03 polymer to ion channel modulator derivative. In one embodiment, the polymer comprises HA covalently linked to a capsaicin or dihydrocapsaisin derivative in a mass:mass ratio of about 1:0.2 polymer to ion channel modulator derivative. In another embodiment, the polymer comprises HA covalently linked to a resiniferatoxin derivative in a mass:mass ratio of about 1:0.03 polymer to ion channel modulator derivative. In another embodiment, the polymer comprises or consists of HA covalently linked to the ion channel modulator derivative, wherein the ion channel modulator comprises or consists of a capsaicin or dihydrocapsaisin derivative or a resiniferatoxin derivative in a mass:mass ratio of between about 1:0.001 and about 1:0.5 polymer to ion channel modulator derivative. In another embodiment, the polymer(s) is/are covalently linked to the ion channel modulator derivative(s) in a mass:mass ratio of between about 1:0.001 and about 1:0.5 polymer(s) to ion channel modulator derivative(s).

In one embodiment, the polymer is mixed with the ion channel modulator derivative in a mass:mass ratio of about 1:0.2 polymer to ion channel modulator derivative. In another embodiment, the polymer is mixed with the ion channel modulator derivative in a mass:mass ratio of about 1:0.03 polymer to ion channel modulator derivative. In one embodiment, the polymer comprises HA mixed with a capsaicin or dihydrocapsaisin derivative in a mass:mass ratio of about 1:0.2 polymer to ion channel modulator derivative. In another embodiment, the polymer comprises HA mixed with a resiniferatoxin derivative in a mass:mass ratio of about 1:0.03 polymer to ion channel modulator derivative. In another embodiment, the polymer comprises or consists of HA mixed with the ion channel modulator derivative, wherein the ion channel modulator comprises or consists of a capsaicin or dihydrocapsaisin derivative or a resiniferatoxin derivative in a mass:mass ratio of between about 1:0.001 and about 1:0.5 polymer to ion channel modulator derivative. In another embodiment, the composition may comprise polymer(s) mixed with the ion channel modulator derivative(s) in a mass:mass ratio of between about 1:0.001 and about 1:0.5 polymer(s) to ion channel modulator derivative(s).

In another embodiment, the polymer is mixed (non-covalently bound) with and covalently linked to the ion channel modulator derivative in a mass:mass ratio of about 1:0.2 polymer to ion channel modulator derivative. In another embodiment, the polymer is mixed (non-covalently bound) with and covalently linked to the ion channel modulator derivative in a mass:mass ratio of about 1:0.03 polymer to ion channel modulator derivative. In one embodiment, the polymer comprises HA mixed (non-covalently bound) with and covalently linked to a capsaicin or dihydrocapsaisin derivative in a mass:mass ratio of about 1:0.2 polymer to ion channel modulator derivative. In another embodiment, the polymer comprises HA mixed (non-covalently bound) with and covalently linked to a resiniferatoxin derivative in a mass:mass ratio of about 1:0.03 polymer to ion channel modulator derivative. In another embodiment, the polymer comprises or consists of HA mixed (non-covalently bound) with and covalently linked to the ion channel modulator, wherein the ion channel modulator comprises or consists of a capsaicin or dihydrocapsaisin derivative or a resiniferatoxin derivative in a mass:mass ratio of between about 1:0.001 and about 1:0.5 polymer to ion channel modulator derivative. In another embodiment, the composition may comprise polymer(s) mixed (non-covalently bound) with and covalently linked to the ion channel modulator derivative(s) in a mass:mass ratio of between about 1:0.001 and about 1:0.5 polymer(s) to ion channel modulator derivative(s).

### Composition Properties

The composition may be in the form of a gel, colloid, suspension, or solution. In one embodiment, the composition is a gel, for example as formed by the polymer.

The composition may have a zeta-potential in the range of −50 mV to +50 mV. The gel may have a viscosity of between 1 and 1,000,000 mPa·s.

Advantageously, the provision of the composition in the form of a gel may provide the additional benefit of providing similar properties, such as viscosity and rheology to natural joint synovial fluid and HA, which can contribute to joint function, joint pain relief and sustained pain relief, where the molecule takes longer to be removed and broken down from the joint.

### The Carrier and Other Additives

The carrier may be a pharmaceutically acceptable carrier. It may be a liquid carrier, which could be aqueous, organic, or multiphasic, or it may be solid in nature. The carrier can also take the form of a gel or a paste. In one embodiment, the carrier is an aqueous carrier comprising water or saline or buffer or buffered saline. Nonlimiting examples of the carrier are phosphate buffered saline PBS, 2-(N-morpholino) ethanesulfonic acid (MES) buffer, phosphate buffer, histidine buffer, acetate buffer, or combinations thereof and aqueous solutions including cosolvents such as polyethylene glycol (PEG) of varying sizes. A person having skill in the art would be able to choose other buffers for use as carrier as well. Generally, a pH range of 4.0-7.4 is preferable. Additionally, the carrier may be formulated to achieve an osmolality that is physiologically compatible, typically in the range of 280-320 mOsm/kg. The carrier may further comprise a further active agent. The active agent may be a therapeutically, prophylactically or diagnostically active substance. The active agent may be a bioactive substance. The active agent may be selected from the group comprising a drug, pro-drug, peptide, protein, and nucleic acid, or combinations thereof. The active agent may comprise or consist of a biomolecule.

The composition may be formulated for administration by a pharmaceutically or cosmetically acceptable route such as by injection or by topical application. The composition may further comprise pharmaceutically acceptable excipients. The composition may further comprise excipient(s) selected from the group consisting of pharmaceutically acceptable salts, polysaccharides, peptides, proteins, amino acids, synthetic polymers, natural polymers, and surfactants.

### The Sterilisation

The conjugate may be sterilized using pharmaceutically acceptable sterilization techniques to ensure safety and efficacy for clinical or cosmetic use. In one embodiment, the conjugate is sterilized by filtration, a widely accepted method for removing contaminants, including bacteria and particulate matter. This is achieved by passing the conjugate solution through a 0.22 μm filter.

In another embodiment, the conjugate may be sterilized by autoclaving. Autoclaving involves exposing the conjugate to steam under pressure at temperatures between 121° C. and 134° C. for a specified period. Gamma irradiation is another sterilization method that may be employed, particularly for some of our formulations that are not amenable to heat-based sterilization techniques. Gamma irradiation offers the advantage of sterilizing both liquid and solid forms of the conjugate without the need for high temperatures. In another embodiment, the conjugate may be freeze-dried (lyophilized) and reconstituted in a sterile environment before use. This approach helps preserve the conjugate's stability during long-term storage while allowing for easy sterilization of the reconstitution solution before administration. In certain embodiments, the sterilization process may also involve aseptic manufacturing techniques. Aseptic processing ensures that the conjugate remains sterile throughout its preparation and packaging, minimizing the risk of contamination during production.

### The Biological Target

In one embodiment, the composition is for localised modulation of nerve cell activity. Nerve cells may comprise cortical neurons, efferent neurons such as motor neurons, or afferent neurons such as pain-sensing nerves cells including dorsal root ganglion neurons (DRGs).

### The Subject

The subject may be a mammal. The subject may be human. The subject may have abnormal physiology/anatomy including tissue damage or tissue degeneration. The subject may have a bone fracture or degeneration. In one embodiment, the subject has osteoarthritis, for example of a joint, such as the knee, hip, shoulder, elbow or the joints between the various (meta) carpal/(meta) tarsal and phalangeal bones.

The subject may be suffering from increased neural activity selected from pain, hypertension, hyperhidrosis, and involuntary muscle spasms including dystonia. The subject may be suffering from spasticity, migraine, dystonia or essential tremor. The subject may be suffering from a pain, which is not neuropathic pain. The subject may be suffering from nociceptive pain. The subject may be suffering from a condition that requires modulation of excitable tissues such as neural and/or muscle cells including cardiac arrhythmias or skeletal muscle overactivity.

The subject may be an individual having knee replacement surgery or another orthopaedic surgery on a joint or bone. The subject may be an individual with post soft tissue surgery.

The subject may be suffering from pain from neuropathic pain, painful bladder syndrome, cystitis, or vaginal atrophy.

In another embodiment, the subject may have normal physiology/anatomy which it is desirable to modify, for example if undergoing or in need of cosmetic treatment.

### Administration

In one embodiment, the composition is, or is arranged to be, administered to subject by local injection, for example at the site of a pain, or infection, such as in a joint or tissue or at close vicinity of the site of pain or infection.

The administration may be targeted to a specifically targeted nerve or muscle tissue. The target nerve may be an afferent nerve associated with a target site, the target site and associated afferent nerve being selected from:
Knee Joint-Genicular nerve or branches thereof;
Hip Joint-Obturator nerve or branches thereof; Shoulder Joint-Suprascapular nerve or branches thereof (most common for shoulder pain);
Elbow Joint-Radial nerve and Ulnar nerve or branches thereof; and Finger Joints-Superficial radial nerve; or combinations thereof.

The nerves may be located in any tissue. Any suitable nerve or nerve fibres may be targeted for pain relief and/or muscle/motor neuron inhibition and/or modulation of function. In one embodiment, the targeted nerve may be a peripheral nerve. The nerve may be a sensory neuron. In addition, any suitable muscle may be targeted for modulation of function, for example cardiomyocytes.

The composition may be administered by injection, with an injection volume of about 1-10 mL in humans, for example when administered to the intra-articular space of the knee joint. Preferably the injection volume is 1-5 mL, and more preferably 2-4 mL, such as 3 mL. Additionally, or alternatively the composition may be administered at a dose in the range 1 mg/mL-30 mg/ml with respect to concentration of polymer. In one embodiment, the composition is administered with a dose of about 1 μg to 100 mg with respect to dose of ion channel modulator derivative(s).

According to another aspect of the invention, there is provided a method of manufacturing the composition according to the invention, the method comprising the steps of covalently bonding ion channel modulator derivative(s) to the polymer and forming a gel, colloid, or solution in a carrier.

According to another aspect of the invention, there is provided a method of manufacturing the composition according to the invention, the method comprising the steps of preparing a derivative of the ion channel modulator(s), covalently bonding the previously prepared derivative to the polymer and forming a gel, colloid, or solution in a carrier.

According to another aspect of the present invention, there is provided a composition suitable in a method of treatment of a subject for pain or a motor neuron disorder selected from spasticity, migraine, dystonia or essential tremor, the method comprising the local administration of the composition to a subject at the site of the pain or motor neuron disorder, wherein the composition comprises a polymer in a carrier, wherein the polymer comprises one or more ion channel modulator derivative(s) covalently bound to the polymer and optionally additionally ion channel modulator derivative(s) mixed with the polymer.

The composition may be administered to the site of infection using local delivery systems including via injection, via instillation, or by topical routes. The composition may also be administered as a prophylactic treatment to prevent pain or other undesirable outcome from the inappropriate activity of excitable tissues; e.g., peri-surgical and post-surgical use.

In one embodiment, the pain is any pain associated with the peripheral nervous system (i.e., not the central nervous system). In one embodiment, the pain may a pain from neuropathic pain, nociceptive pain, painful bladder syndrome, cystitis, or vaginal atrophy.

The pain may be joint pain, such as from osteoarthritis, or tissue pain, for example from tissue damage, disease, or infection. The pain may typically be chronic pain but may also be acute pain.

The composition may be provided locally in a treatment, for example by injection at the site of the pain or motor neuron to be modulated. Migraine pain may be treated by localised injection into the forehead and/or temple of a subject.

According to another aspect of the present invention, there is provided the use of the composition according to the invention for pain relief or motor neuron modulation in a subject.

According to another aspect of the present invention, there is provided the use of the composition according to the invention for modulation of nerve cell activity, in a subject. The subject may be afflicted with a disorder associated with nerve cell activity.

According to another aspect of the present invention, there is provided a kit comprising the composition according to the invention, and a syringe. The composition may be pre-loaded in the syringe. The kit may further comprise instructions for local administration of the composition to a site of pain, such as a joint, or a site for motor neuron blockade/modulation.

### Ion Channel Modulator Dose Treatment

According to an aspect of the invention, there is provided the use of the composition comprising one or more ion channel modulators, such as TRPV1 modulators, for the localised modulation of nerve cell activity, in a subject, wherein the treatment comprises the administration of the ion channel modulator derivative(s) to the subject by localised injection into a tissue or joint at the site of the pain, and optionally wherein the ion channel modulator derivative(s) are administered at a dose of 1 μg to 100 mg in a 1 to 10 mL volume. The administration may be local administration.

The ion channel modulator(s) may act on any of the relevant ion channels in excitable tissues, such as TRPV. These ion channel modulators are therapeutically effective at specific concentrations; however, the therapeutic window can be narrow. At higher concentrations, they may exhibit safety signals, including toxicity and other adverse events—for example, cardiac toxicity with Nav and Cav blockers, muscle weakness with Cav blockers, and acute pain with TRPV1 agonists. More specifically, TRPV1 agonists can cause acute pain, burning sensations, and discomfort at higher concentrations due to the initial activation of the channel, which triggers pain perception before desensitization occurs. Through a sustained and monitored release mechanism, the present invention establishes a safe and effective concentration of the ion channel modulator in vivo to modulate excitable cell activity without causing unacceptable adverse events. The conjugation of the ion channel modulator or a derivative to the polymer results in a depot of the modulator that is released over time in vivo, maintaining a profile within the therapeutic range. This approach reduces the safety issues associated with bolus dosing and provides an extended duration of action.

The present invention allows for the use of higher doses or concentrations of ion channel modulators compared to previous therapeutics, due to the conjugation of the modulators to a biocompatible polymer. This conjugation stabilizes the modulator, reduces its systemic toxicity, and enables a sustained release profile, preventing the rapid spikes in concentration that can lead to adverse events. As a result, the system allows for higher therapeutic concentrations while maintaining safety, offering extended duration of action and improved control over the modulator's bioavailability.

### BRIEF DESCRIPTION OF THE DRAWINGS

FIGS. 1 (a) and 1 (b). Zeta-potential is measure of the effective electric charge on the nanoparticle's surface, quantifying the charges. The mean zeta potential of sodium hyaluronate (1 mg/mL) was recorded at −23.2 mV and is shown in FIG. 1A. The mean zeta potential of the compound obtained in example 11 (namely (E)-2-methoxy-4-[(8-methylnon-6-enamido)methyl]phenyl 3-aminopropanoate-introduced sodium hyaluronate) (1 mg/mL) was recorded at −30.4 mV and shown in FIG. 1B. These results prove that the solution of compound example 11 is highly anionic and when compared with sodium hyaluronate, the composition has a higher negative value. A zeta potential of range +/−60 mV indicates stability in the case of colloid systems. In the case of the solution of compound example 11, while conjugation with capsaicin derivative may theoretically be expected to reduce the negative charge, surprisingly the opposite effect was seen to occur. This increased negative value compared to sodium hyaluronate may be due to a compaction of the molecule, leading to increased charge density on the surface. While conjugation with a capsaicin derivative might theoretically be expected to reduce the negative charge, the observed increase in zeta potential suggests that structural rearrangement or compaction could be concentrating the negative charge. The broadening of the zeta potential peak for the compound example 11 likely reflects increased heterogeneity in the surface charge distribution due to the conjugation process, including variations in conjugation efficiency, surface charge shielding, and particle size changes.

FIG. 2. The graph illustrates the release kinetics of exemplary compositions A and B under different conditions (4° C. and 37° C.). Compositions are prepared at 1 mg/mL and the levels of free capsaicin assayed using patch clamp at various timepoints. The results show the compositions to have good stability at 4° C. with no significant capsaicin release over a >7-day time-course. Both Compositions A and B show release of capsaicin over 12.5% of the conjugated load by day 3 at 37° C. with agitation, and over 50% by day 16. Composition A shows faster release kinetics compared to Composition B. Neither composition showed a burst release. The in vitro kinetic release profile aligns with the expected in vivo behaviour, minimizing initial sensitization through an effective lower initial dose compared to a bolus administration. Pain relief is provided by the sustained release of the ion-channel modulator from the composition, maintaining therapeutic levels and extending the duration of action.

FIG. 3. The graph illustrates the release kinetics of exemplary compositions A and D in modelled in vivo conditions. The levels of free capsaicin were assayed using patch clamp at various timepoints up to approximately 10 days. The results show release of capsaicin of over 50% of the conjugated load by day 10 at 37° C. Further, the release kinetics of both compositions are not significantly different.

FIG. 4. The graph illustrates the release kinetics of exemplary composition E in modelled in vivo conditions. The levels of free capsaicin were assayed using patch clamp at various timepoints up to approximately 10 days. The results show release of capsaicin of over 50% of the conjugated load by day 7 at 37° C. with agitation in synovial fluid. The results demonstrate favourable release kinetics under in vivo conditions (37° C. with agitation).

FIG. 5. The graph illustrates the release kinetics of exemplary compositions F and G in modelled in vivo conditions. The levels of free capsaicin were assayed using patch clamp at various timepoints up to approximately 10 days. The results show slower capsaicin release under the same conditions of 37° C. with agitation in synovial fluid than those of Compositions A and D particularly (FIG. 2) over the first 100 hours. This shows the advantage of exploring several derivatives of capsaicin conjugated to HA in tuning and slowing down release of free capsaicin. The results demonstrate favourable release kinetics under in vivo conditions (37° C. with agitation).

FIG. 6. The graph illustrates the duration of immobility (nocifensive behaviour) displayed by the animals for the first 15 minutes following intra-articular treatment administration. The results show that HA+(E)-CAP mixture and exemplary composition A led to a duration of immobility (as an index of pain) equivalent to that of control (vehicle treatment), and less than that of free capsaicin. This result demonstrates the benefit of conjugating capsaicin in reducing the initial acute period of sensitisation.

FIG. 7. The graph illustrates the results for exemplary composition A in the VF test. Following administration of treatments, VF test was performed in naïve rats to measure paw withdrawal threshold (PWT) at the ipsilateral hind paw. The area under the curve (AUC) was calculated over time post-treatment with respect to baseline. The results are shown at +2.5 h (left side columns) and +24 h (right side columns) post-treatment. In each set of columns from left to right test materials are Vehicle, HA (sodium hyaluronate), CAP (free (E)-capsaicin), CAP+HA (free (E)-capsaicin+HA), and exemplary composition A (capsaicin derivative conjugated to HA) at three different concentration levels (×1, ×5, ×10 that of the CAP+HA concentration level). The results show that the exemplary composition A has a higher PWT compared to both free (E)-capsaicin (CAP) and the (E)-capsaicin+HA (CAP+HA) mixture groups, demonstrating the benefit of conjugation of the capsaicin derivative to HA in allowing loading of up to 10 times the levels of free (E)-capsaicin without the same level of acute sensitisation (i.e. pain) as capsaicin alone (CAP) or the mixture group (CAP+HA).

FIG. 8. The graph illustrates the results for number of nociceptive responses (flinching, licking, shaking) displayed by the MIA rats for the first 20 minutes following intra-articular treatment administration. The results show that the exemplary composition A (Table 3) at three different concentration levels (×1, ×3, and ×5 that of free (E)-CAP concentration level) led to reduced pain-related responses that is equivalent to that of control (vehicle treatment) and less than that of free (E)-capsaicin. This result demonstrates the benefit of conjugating capsaicin derivative to HA in lowering the initial acute period of sensitisation (i.e. pain) in the MIA model of knee OA pain.

FIG. 9. The graph illustrates the results for exemplary composition A (Table 1) in the WB test. Following MIA injection, the weight bearing on the ipsilateral (injured) hind limb was reduced indicating weight bearing asymmetry as a measure of spontaneous joint pain. The results show that following intra-articular treatment administration, the exemplary composition A improved this weight bearing deficit in the MIA rats, indicating alleviation of knee OA-related joint pain, for up to 4 weeks compared to free (E)-Capsaicin (CAP) with effect up to 2 weeks post-treatment. This result demonstrates the benefit of conjugating capsaicin derivative to HA in achieving a prolonged, sustained pain-relieving effect that persists longer than capsaicin alone (CAP).

### DETAILED DESCRIPTION OF THE INVENTION

### Definitions

A “gel” is understood to be a semi-solid matrix of polymers, which exhibits no flow when in the steady-state, although the continuous phase may still diffuse through this matrix.

The term “colloid” is understood to mean a dispersion of polymer and peptides in a carrier. The colloid is understood to be liquid in form.

The term “solution” is understood to mean the polymer and/or peptides are dissolved in a solvent carrier.

The term “biocompatible” is understood to include non-toxic to the human or animal body. To be biocompatible, the composition may not cause an immune response.

The term “biodegradable” is understood to include the ability to breakdown over time in the tissue or body of a human or animal, and/or in the environment. The time for complete degradation may be at least 1 week, at least 1 month, at least 2 months, at least 6 months, or at least 12 months. The time for complete degradation may be no more than 12 months. The time for complete degradation may be no more than 6 months.

The term “localised”, “local delivery”, “local administration” or “localised treatment” or similar, is intended to refer to the administration of treatment directly to a site or into the vicinity of the site of the pain or site of relevant pathology, for example by injection into the effected tissue, or directly into the afflicted joint. For delivery into a joint, the composition may be administered into a joint space, such as into the synovial fluid. The treatment may not be administered systemically.

A “covalent bond”, also called a molecular bond, is a chemical bond involving the sharing of electron pairs between atoms. These electron pairs are known as shared pairs or bonding pairs, and the stable balance of attractive and repulsive forces between atoms, when they share electrons, is known as covalent bonding.

The term “inhibition” used herein may be a total or partial inhibition of activity, such as channel activity or electrical activity.

The term “modulation” used herein may be a total or partial inhibition of activity or total or partial upregulation of activity or a change in the pattern of activity such as channel activity or electrical activity.

### EMBODIMENTS

Embodiments of the invention will now be described in more detail in light of non-limiting experiments. The embodiments mentioned in this text relate, where applicable, to all aspects of the invention, even if this is not always separately mentioned. It is understood that optional features of one embodiment or aspect of the invention may be applicable, where appropriate, to other embodiments or aspects of the invention.

This disclosure shows potential for a composition comprising a medically approved biodegradable polymer covalently bonded with ion channel modulators or derivatives provide a reduction of potential safety signals, efficacy in terms of desired neural modulation, and an elongated residency time for the ion channel modulator.

One aspect of the present disclosure comprises taking a medically approved biodegradable and endogenous polymer, such as hyaluronic acid or a salt form thereof, chemically modified to have properties that modulate the activity of excitable cells such as neurons. One method to achieve this is to attach ion channel modulator(s) or derivatives to the polymer chains forming, or comprised in, the biodegradable polymer. Upon conjugation, the ion channel modulator or derivative becomes covalently bound to the polymer, resulting in a single chemical entity. This entity forms a hybrid molecule, where the biodegradable polymer backbone (e.g., hyaluronic acid or a salt form) serves as a carrier and stabilizing matrix. While the modulator or derivative is attached, it loses its functional ability to interact with specific ion channels on excitable cells. The chemical bonding between the polymer and modulator or its derivative creates a stable conjugate that controls the release profile of the modulator in vivo, ensuring it remains within therapeutic levels for an extended period. This single entity, comprised of both the polymer and modulator derivative, retains the biocompatibility and biodegradability of the polymer, while the pharmacological activity of the ion channel modulator is restored once released via hydrolysis.

One group of ion channel modulators capable of modulating nerve activity are TRPV1 agonists, such as capsaicin and resiniferatoxin. It is well reported in the literature that the mechanism by which this group works involves the persistent activation of TRPV1 receptors, leading to an initial excitation followed by a long-term desensitization. This results in an initial increase in pain sensation (experienced as warming, tingling, stinging, or burning) followed by a loss of pain sensation. Advantageously, TRPV1 receptors are primarily found on small pain-sensing fibres, making them an attractive target for candidate analgesics. However, in the treatment of pain, TRPV1 agonists are often limited by the initial excitation, which can necessitate the use of concomitant analgesics, such as opioids, to manage the acute pain response. Furthermore, while the pain relief from TRPV1 agonists is long-lasting, the effect diminishes over time, raising the potential for breakthrough pain. These challenges highlight problems that remain to be solved with this approach. In this application, we have addressed the limitations of the TRPV1 agonists and developed novel compounds and methods that provide the following key advantages:
(1) reduced initial excitation of TRPV1 receptors: by conjugation of the ion channel modulator derivatives to a biocompatible polymer, we have been able to minimize the acute pain response typically associated with TRPV1 agonists. This ensures a more tolerable experience for the patient, reducing the need for concomitant analgesics such as opioids to manage the initial discomfort.
(2) slow release of ion channel modulators in target tissues: through a controlled sustained release mechanism, the ion channel modulators are gradually delivered to the target tissues. This not only extends the duration of therapeutic effects but also maintains the concentration of the modulator within the therapeutic window for a prolonged period, reducing the risks associated with bolus dosing.
(3) pain attenuation through gradual release: the gradual release of the modulator leads to progressive desensitization of TRPV1 receptors, resulting in a steady reduction in pain without the sharp fluctuations that can lead to breakthrough pain. This ensures more consistent pain relief over time and reduces the likelihood of pain returning before the next dose is administered.

The composition according to this invention contains one or more ion channel modulator derivatives either covalently bound and alternatively or additionally mixed with biodegradable polymer(s). In clinical use, the composition may be injected into the target area nearby the nerves of interest, where gradual and controlled release of the ion channel modulator(s) can be tailored in order to achieve:
Sustained exposure of the pain sensing neurons to ion channel modulators such as TRPV1 agonists, but at a level low enough not to illicit the initial excitation and pain.
Sustained release over a prolonged period to ensure therapeutic cover over the entire treatment interval reducing the risk of breakthrough pain.

When the TRPV1 ion channel modulator is derivatized to enable covalent linkage to the polymer via ester bonds, amide, carbamate, or carbonate bonds, this chemical modification results in a total potency loss, leading to a marked decrease in its ability to activate the TRPV1 ion channel. This reduction in activity occurs because the derivatization alters non-permanently the modulator's structure, preventing it from effectively interacting with its binding site on TRPV1. The conjugation of the derivatized modulator to the polymer further inactivates its biological function, resulting in a compound that lacks any appreciable agonist activity on TRPV1. However, when the conjugate is exposed to modelled in vivo or in vivo conditions, such as at physiological pH (above 7), body temperature (37° C.) and in the presence of specific enzymes (e.g., hyaluronidase, peptidase, and esterase), a sustained release of the TRPV1 modulator occurs through the hydrolysis of the covalent bonds connecting it to the polymer. Once liberated into the biological environment, the free TRPV1 modulator regains its full agonist activity, allowing it to efficiently interact with the TRPV1 receptor.

Since the activation of voltage gated channels such as TRPV1 channels on pain sensing neurons is a necessary step for nerve conduction, the composition will reduce, or optionally effectively block, all pain signals from the nerve. Other channels exist that may act as a target and would give a similar overall effect on the nerve or excitable tissue, such as Nav and Cav channels. In one embodiment combination of different ion channel modulators may be used in the composition.

Additional chemical design and optionally modification of the biodegradable polymer to attach ion channel modulator derivative molecules can be achieved by the following method:

The design of the polymer ensures that the ion channel modulators can be released in a controlled manner and diffuse to their active site (i.e., the relevant ion channel) on nerve cells. The controlled release can be influenced by the size of the polymer chains. Typically, polymer chains for biodegradable polymers can range from 1 kDa to several MDa. Control of polymerization chain lengths has been reported in the scientific literature. For example, the polymer chain length of hyaluronic acid can be monitored by methods such as control of temperature with appropriately genetically engineered bacteria, allowing for precise tuning of its biodegradability and release properties.

### Degradation of the Biodegradable Polymer In Vivo

The biodegradable polymer can be designed to degrade fully in vivo from 0.5 months to 5 years, more preferably from 5 months to 3 years, and most preferably from 6 months to one year. Degradation can be controlled and tailored to ensure the required degradation times are achieved. The main factors that determine the degradation process are the chemical structure of the polymer chain in the polymer backbone; hydrophilic/hydrophobic groups; polymer morphology; molecular weight; surface area; catalysts; and additives of resorbable polymers. The chemical structure of the polymer chain in the polymer backbone is the most important parameter. Generally, carbonates degrade faster than the ester group, which in turn degrades faster than the amide group. Based on this, it is possible to predict degradation of a given polymer. The hydrophilic/hydrophobic character is another important parameter in polymer degradation. Poly(glycolic acid) degrades faster than the more hydrophobic poly(lactic acid), although they have the same degradable chain in the polymer backbone. The hydrophilic/hydrophobic character is another important parameter in polymer degradation, as it directly influences the rate of degradation and the interaction with biological tissues. Hyadd is a family of chemically modified hyaluronic acid (HA) derivatives that incorporate hydrophobic alkyl chains, specifically designed to improve the viscoelastic properties and biodegradation profile of pristine hyaluronic acid for applications such as joint lubrication. The chemical modifications of Hyadd with alkyl chains adjust the polymer's balance between hydrophilic and hydrophobic components, improving its mechanical stability, resistance to degradation, and biocompatibility. The viscoelastic properties of Hyadd allow it to retain the beneficial characteristics of HA, such as lubrication and shock absorption, while extending its residence time in tissues like the synovial fluid in joints. The degradation rate is further influenced by polymer morphology. Crosslinkers can be used to slow down the degradation of hyaluronic acid (HA). These crosslinkers bind HA polymer chains to each other, both intra- and inter-molecularly, forming a polymer network. This network is more stable and, physiochemically, appears more viscous at a given concentration. This process transforms a viscous liquid into a gel, creating a physical and chemical barrier against enzymatic activity and free radical breakdown. Since the gel network is connected at numerous points, enzymes and free radicals can only degrade the polymer in much smaller sections at a time. Additionally, due to the large size of enzymes, they may have difficulty penetrating the gel structure, further contributing to the slower degradation of the polymer. This translates into longer persistence of the gel of the present invention at a target site in vivo. Common crosslinkers include 1,4-butanediol diglycidyl ether (BDDE), and di-vinyl sulfone (DVS). Both react with hydroxyl sites on the HA chains and offer similar results in slowing down enzymatic and free radical degradation of HA gels once injected. BDDE may be used as a crosslinker, binding together two HA polymer chains, transforming liquid HA solutions into gels. The primary hydroxyl group (—CH₂OH) within the HA monomeric unit is the main target for reaction with BDDE, though the secondary hydroxyl groups (—CHOH) may also participate under certain conditions.

### Production of Hyaluronic Acid Gel

### a) Crosslinking of Hyaluronic Acid

A representative method for cross-linking of HA:

HA was first dissolved in 1% NaOH at a concentration of 10% w/w, after which BDDE was added to the HA solution with vigorous stirring. The final concentrations of BDDE were 0.4% v/v, 0.6% v/v, 0.8% v/v and 1% v/v, respectively. The solution was then allowed to crosslink at 40° C. for 5 h, followed by being dried at room temperature for 3 days. Phosphate buffered saline (PBS: NaCl, 9 mg·mL⁻¹; KH₂PO₄, 0.03 mg·mL⁻¹; Na₂HPO₄·2H₂O, 0.14 mg·mL⁻¹; pH 7.0) of 500 mL was then added to the above crosslinked HA to make it swell, after which it was put into dialysis bag and dialysed sequentially with excessive deionised (DI) water and PBS to remove the residual BDDE. The resulting gel was adjusted with PBS to obtain a gel with a HA concentration of 20 mg·mL⁻¹ and then used in a homogenizer to obtain gel particles of 0-400 nm in size. Before being used, the obtained injectable gel was sterilized in a high-pressure steam sterilizer set at 120° C., 20 min. When the biodegradable polymer is hyaluronic acid, the hyaluronic acid used for covalent linking with the ion channel modulator derivative(s) can be used in a non-crosslinked form, or can be crosslinked, optionally as disclosed above.

The half-life of hyaluronic acid injected in vivo non-crosslinked is of the order of hours-days, while cross-linking has been shown to increase residence time to months using cross-linking agents like BDDE.

### b) Curing of the Gel In Situ

The gel may be designed to be cured in situ. This gives the added benefit to ensure the gel does not migrate from the target site and would also give the gel additional mechanical strength. It is difficult to inject very viscous materials; however, a low viscosity polymer solution could be injected to the target nerve site and by curing in situ the gel can cross-link and form a strong solid support of high mechanical strength. In situ curing of hyaluronic acid (HA) can be achieved using ionic cross-linkers or through the use of thermo-responsive or pH-sensitive gels, which form a stable gel upon injection under physiological conditions, or via photo-cross-linking, where an external light source induces cross-linking in the presence of a photosensitive agent.

### Administration of the Gel

### a) Targeting the Gel to Different Nerve Fibres or Excitable Cells

The gel may be formulated to selectively target different nerve fibres or other excitable cells. This can be achieved by a concentration effect or by increasing the volume. For example, C-5 type pain fibres are non-myelinated and of 0.2-1.5 micron diameter while A-delta pain fibres are myelinated and of 1-5 micron diameter. These pain fibres are smaller in diameter compared to afferent or motor fibres. This differing anatomy offers the opportunity to design gels or ligands that selectively target pain fibres without interfering with larger nerve types, thereby minimizing potential adverse effects. For instance, TRPV1 channels are selectively expressed on these small-diameter fibres, making them a natural target for the gel formulation.

### b) Delivery of the Gel to Target Site

The gel may be delivered to the site of action (the vicinity of the target nerve/excitable tissue) using a needle attached to a syringe loaded with the gel, or by another system suited to localised delivery. A range of needles are available depending on the intended site of action, viscosity of the gel, and other factors. The injection volume can be dispensed in the usual fashion—i.e., navigation to site of administration and delivery of the gel as a bolus. Alternatively, to facilitate delivery of small amounts of gel in a relatively wide area encompassing the target nerve, the needle can have a mechanism that delivers aliquots of gel as a series of micro-injections. Initially, the clinician delivers the needle to a central area at which point a mechanism is engaged that causes the needle tip to partially retract/move/inject over a specified automated cycle to result in optimal widespread delivery of the gel.

A person skilled in the art will appreciate that different grades of gel with differing types and amount of ion channel modulator derivative(s) can be designed for different target sites and for larger/smaller nerves. For example, the trigeminal nerve branches have quite distinct dermatomes, meaning targeting of a given branch should allow targeting of effect to the respective dermatome. More extensive targeting may require delivery of ion channel modulator derivative(s) containing gel to the trigeminal ganglion for widespread effect, or alternatively separate targeting of each branch.

A similar set of delivery systems could be employed where the target site is not suitable for needle administration. For instance, a cannula or catheter could be used for instillation into the bladder, enabling the targeted delivery of the composition to mucosal surfaces without invasive penetration. Additionally, topical formulations such as paints, lotions, creams, gels, or sprays could be developed for direct application to external areas like the skin or mucous membranes. These forms allow for localized delivery of the active compound, ensuring that the treatment can reach the affected tissues without systemic exposure.

The composition for treating pain as disclosed in this disclosure comprises a TRPV1 ion channel modulator derivative, capable of covalently linking to a polymer, wherein the ion channel modulator derivative is covalently linked to a polymer, and alternatively or additionally the ion channel modulator derivative is mixed with the polymer. The TRPV1 ion channel modulator derivative may be any one of (E)-2-methoxy-4-[(8-methylnon-6-enamido)methyl]phenyl glycinate; (E)-2-methoxy-4-[(8-methylnon-6-enamido)methyl]phenyl 3-aminopropanoate; (E)-2-methoxy-4-[(8-methylnon-6-enamido)methyl]phenyl 2-[2-(2 aminoethoxy)ethoxy]acetate; (E)-2-methoxy-4-[(8-methylnon-6-enamido)methyl]phenyl 3-(2-aminoethoxy)propanoate; (E)-2-methoxy-4-[(8-methylnon-6-enamido)methyl]phenyl 2-(2-aminoethoxy)acetate; (E)-2-methoxy-4-[(8-methylnon-6-enamido)methyl]phenyl (5-aminopentyl)carbamate; (E)-2-methoxy-4-[(8-methylnon-6-enamido)methyl]phenyl [2-(2-aminoethoxy)ethyl]carbamate; 2-methoxy-4-{[(E)-8-methylnon-6-enamido]methyl]phenyl trans-4-aminocyclohexane-1-carboxylate; (E)-2-methoxy-4-[(8-methylnon-6-enamido)methyl]phenyl 3-aminopyrrolidine-1-carboxylate; (E)-2-methoxy-4-[(8-methylnon-6-enamido)methyl]phenyl (2-monoethyl)(isopropyl)carbamate; 2-methoxy-4-{[(E)-8-methylnon-6-enamido]methyl}phenyl L-isoleucinate; 2-methoxy-4-{[(E)-8-methylnon-6-enamido]methyl}phenyl trans-4-(2-aminoacetamido)cyclohexane-1-carboxylate; 2-methoxy-4-{[(E)-8-methylnon-6-enamido]methyl}phenyl trans-4-(2-aminopropanamido)cyclohexane-1-carboxylate; (E)-2-methoxy-4-[(8-methylnon-6-enamido)methyl]phenyl (3-aminopropanoyl)-L-prolinate; (E)-2-methoxy-4-[(8-methylnon-6-enamido)methyl]phenyl piperidine-2-carboxylate; (E)-2-methoxy-4-[(8-methylnon-6-enamido)methyl]phenyl glycyl-L-prolinate; (E)-2-methoxy-4-[(8-methylnon-6-enamido)methyl]phenyl 1-glycylpiperidine-2-carboxylate; (E)-2-methoxy-4-[(8-methylnon-6-enamido)methyl]phenyl 1-(3-aminopropanoyl) piperidine-2-carboxylate; (E)-2-methoxy-4-[(8-methylnon-6-enamido)methyl]phenyl 2-[2-(2-aminoethoxy)ethoxy]acetate; (E)-2-methoxy-4-[(8-methylnon-6-enamido)methyl]phenyl L-valinate; (E)-2-methoxy-4-[(8-methylnon-6-enamido)methyl]phenyl (3-aminopropanoyl)-L-valinate; 2-methoxy-4-{[(E)-8-methylnon-6-enamido]methyl}phenyl (1S,2R)-2-aminocyclopentane-1-carboxylate; (E)-2-methoxy-4-[(8-methylnon-6-enamido)methyl]phenyl (3-aminopropanoyl)-D-prolinate; and (E)-2-methoxy-4-[(8-methylnon-6-enamido)methyl]phenyl (3-aminopropanoyl)-DL-prolinate; as their salts or free forms.

Synthesis of the compounds as well as synthesis of compounds where the ion channel modulator derivative is introduced into a polymer is described in the examples below.

### Examples—Chemistry

### Example 1

### Synthesis of (E)-2-methoxy-4-[(8-methylnon-6-enamido)methyl]phenyl glycinate, HCl Salt

To a solution of N-(tert-Butoxycarbonyl)glycine (172 mg, 0.98 mmol, 1.5 eq.) in dichloromethane (10 mL) cooled at 0° C. was added HBTU (448 mg, 1.18 mmol, 1.8 eq.) and acetonitrile (8 mL). The mixture was stirred for 5 minutes before adding triethylamine (272 μL, 1.96 mmol, 3.0 eq.) and a solution of pure (E)-capsaicin (200 mg, 0.66 mmol, 1.0 eq.) in dichloromethane (4 mL). The reaction mixture was stirred at room temperature for 3 hours under a nitrogen atmosphere. Then the solvents are evaporated under reduced pressure and the crude was taken up in EtOAc and water. The organic layer was successively washed with 0.5 M NaOH, brine, 0.5 M HCl & brine, dried over Na₂SO₄, filtered through a silica gel pad and evaporated under reduced pressure to afford the Boc-protected derivative as an oil. The product was directly used in the next step without further purification. The oil was taken up in dichloromethane (4 mL) and EtOAc (4 mL), the solution cooled down to 0° C. before adding 3M HCl in CPME (4 mL, 12 mmol) under nitrogen. The reaction mixture was stirred at room temperature for 4 hours, still under an inert atmosphere. The precipitate that formed was filtered, washed with dichloromethane and Et₂O, then triturated with Et₂O, acetone and Et₂O again to afford the titled compound (227 mg, 84%) as an off-white solid. ¹H NMR (DMSO-d₆, 500 MHz): δ (ppm): 0.93 (d, J=6.6 Hz, 6H), 1.31 (m, 2H), 1.52 (m, 2H), 1.95 (q, J=6.6 Hz, 2H), 2.15 (t, J=7.2 Hz, 2H), 2.21 (m, 1H), 3.76 (s, 3H), 4.13 (m, 2H), 4.26 (d, J=5.4 Hz, 2H), 5.29-5.40 (m, 2H), 6.85 (d, J=7.8 Hz, 1H), 7.05 (bs, 1H), 7.07 (d, J=7.8 Hz, 1H), 8.35 (m, 1H), 8.45 (bs, 3H)

### Example 2

### Synthesis of (E)-2-methoxy-4-[(8-methylnon-6-enamido)methyl]phenyl 3-aminopropanoate, HCl Salt

To a solution of N-(tert-Butoxycarbonyl) β-alanine (186 mg, 0.99 mmol, 1.5 eq.) in dichloromethane (4 mL) cooled at 0° C. was added EDC·HCl (228 mg, 1.19 mmol, 1.8 eq.) in dichloromethane (2 mL). The mixture was stirred at 0° C. for 5 minutes before adding DMAP (32 mg, 0.26 mmol, 0.4 eq.) and a solution of pure (E)-capsaicin (200 mg, 0.66 mmol, 1.0 eq.) in dichloromethane (4 mL). The reaction mixture was stirred at room temperature for 4 hours under a nitrogen atmosphere. Then the solvents are evaporated under reduced pressure and the crude was taken up in a mixture of EtOAc/Et₂O and water. The organic layer was successively washed with 0.5 M NaOH, brine, 0.5 M HCl & brine, dried over Na₂SO₄, filtered through a silica gel pad and evaporated under reduced pressure to afford the Boc-protected derivative as an oil. The product was directly used in the next step without further purification. The oil was taken up in dichloromethane (4 mL) and EtOAc (4 mL), the solution cooled down to 0° C. before adding 3M HCl in CPME (4 mL, 12 mmol) under nitrogen. The reaction mixture was stirred at room temperature for 5 hours, still under an inert atmosphere. The precipitate that formed was filtered, washed with dichloromethane and Et₂O, then triturated with Et₂O, acetone and Et₂O again to afford the titled compound (217 mg, 80%) as an off-white solid. 1H NMR (DMSO-d₆, 500 MHz): δ (ppm): 0.93 (d, J=6.6 Hz, 6H), 1.31 (m, 2H), 1.52 (m, 2H), 1.95 (q, J=6.6 Hz, 2H), 2.15 (t, J=7.2 Hz, 2H), 2.21 (m, 1H), 2.95 (t, J=7.1 Hz, 2H), 3.11 (m, 2H), 3.74 (s, 3H), 4.25 (d, J=5.4 Hz, 2H), 5.28-5.39 (m, 2H), 6.82 (d, J=8.1 Hz, 1H), 7.01 (bs, 1H), 7.06 (d, J=8.1 Hz, 1H), 8.01 (bs, 3H), 8.34 (m, 1H)

### Example 3

### Synthesis of (E)-2-methoxy-4-[(8-methylnon-6-enamido)methyl]phenyl glycinate-Introduced sodium hyaluronate

Sodium hyaluronate with an average molecular weight of 750 kDa (125 mg, 2.5 eq. disaccharide unit) in a mixture of water (16 mL) and dioxane (16 mL) was stirred at room temperature for 1 hour until fully soluble. A solution of N-hydroxysuccinimide (29 mg, 0.25 mmol, 2.0 eq.) in water (2 mL), then a solution of compound obtained in example 1 (50 mg, 0.125 mmol, 1.0 eq.) in water/dioxane (2 mL/2 mL), and finally a solution of EDC·HCl (24 mg, 0.125 mmol, 1.0 eq.) in water (1 mL) were successfully added in that order with 5 minutes stirring between each addition. The reaction mixture was stirred at room temperature for 14 hours before the addition of NaHCO₃ (100 mg) as a solution in water (2 mL). The reaction mixture was stirred again at room temperature for an additional 6 hours before the successive addition of AcOH (30 μL) in water (1 mL) then NaCl (0.75 g). The reaction mixture was stirred at room temperature for 1 hour and poured into 150 mL of EtOH. The mixture was stirred at room temperature for 15 minutes and filtered. The precipitate was washed with ethanol, twice with 85% ethanol in water, twice with ethanol and several times with Et₂O to afford the titled compound as an off-white solid (90 mg after overnight drying). The content of capsaicin was in the 6-10% weight/weight range as determined by ultraviolet spectroscopy.

### Example 4

### Synthesis of (E)-2-methoxy-4-[(8-methylnon-6-enamido)methyl]phenyl 2-[2-(2-aminoethoxy)ethoxy]acetate, TFA salt

To a solution of 2,2-dimethyl-4-oxo-3,8,11-trioxa-5-azatridecan-13-oic acid (828 mg, 3.14 mmol, 1.6 eq.) in dichloromethane (30 mL) cooled at 0° C. was added EDC·HCl (751 mg, 3.92 mmol, 2.0 eq.) in dichloromethane (2 mL). The mixture was stirred at 0° C. for 5 minutes before adding DMAP (144 mg, 1.18 mmol, 0.6 eq.) in dichloromethane (2 mL) and a solution of pure (E)-capsaicin (600 mg, 1.96 mmol, 1.0 eq.) in dichloromethane (8 mL). The reaction mixture was stirred at room temperature for 16 hours under a nitrogen atmosphere. Then the solvents are evaporated under reduced pressure and the crude was taken up in a mixture of EtOAc/Et₂O and water. The organic layer was successively washed with 0.5 M NaOH, brine, 0.5 M HCl & brine, dried over Na₂SO₄, filtered through a silica gel pad and evaporated under reduced pressure to afford the Boc-protected derivative as a yellow oil. The product was directly used in the next step without further purification. The oil was taken up in dichloromethane (20 mL) and the solution cooled down to 0° C. before adding TFA (2.25 mL, 29.4 mmol). The reaction mixture was stirred at room temperature for 5 hours under a nitrogen atmosphere. The mixture was diluted with methanol, filtered and evaporated. The oil was taken up in methanol and evaporated two additional times to afford the titled compound (1.17 g, quantitative) as light orange oil after further drying under high vacuum. 1H NMR (DMSO-d₆, 500 MHz): δ (ppm): 0.93 (d, J=6.6 Hz, 6H), 1.31 (m, 2H), 1.52 (m, 2H), 1.94 (q, J=6.6 Hz, 2H), 2.14 (t, J=7.3 Hz, 2H), 2.21 (m, 1H), 2.99 (m, 2H), 3.60-3.64 (m, 4H), 3.71 (m, 2H), 3.75 (s, 3H), 4.25 (d, J=5.4 Hz, 2H), 4.42 (s, 2H), 5.29-5.39 (m, 2H), 6.82 (dd, J=8.1 Hz, J=1.7 Hz, 1H), 7.01 (d, J=1.7 Hz, 1H), 7.03 (d, J=8.1 Hz, 1H), 7.75 (bs, 3H), 8.32 (m, 1H).

### Example 5

### Synthesis of (E)-2-methoxy-4-[(8-methylnon-6-enamido)methyl]phenyl 3-aminopropanoate-introduced sodium hyaluronate

Sodium hyaluronate with an average molecular weight of 750 kDa (500 mg, 2.5 eq. disaccharide unit) in a mixture of water (60 mL) and dioxane (60 mL) was stirred at room temperature for 1 hour until fully soluble. A solution of N-hydroxysuccinimide (116 mg, 1.00 mmol, 2.0 eq.) in water (2 mL), then a solution of compound obtained in example 2 (208 mg, 0.5 mmol, 1.0 eq.) in water/dioxane (1 mL/3 mL), and finally a solution of EDC·HCl (96 mg, 0.5 mmol, 1.0 eq.) in water (3 mL) were successfully added in that order with 5 minutes stirring between each addition. The reaction mixture was stirred at room temperature for 16 hours before the addition of NaHCO₃ (400 mg) as a solution in water (6 mL). The reaction mixture was stirred again at room temperature for an additional 6 hours before the successive addition of AcOH (120 μL) in water (4 mL) then NaCl (3.0 g). The reaction mixture was stirred at room temperature for 1 hour and poured into 600 mL of EtOH. The mixture was stirred at room temperature for 15 minutes and filtered. The precipitate was washed with ethanol, twice with 85% ethanol in water, twice with ethanol and several times with Et₂O to afford the titled compound as a white meringue (400 mg after overnight drying). The content of capsaicin was in the 1-5% weight/weight range as determined by ultraviolet spectroscopy.

### Example 6

### Synthesis of (E)-2-methoxy-4-[(8-methylnon-6-enamido)methyl]phenyl 3-(2-aminoethoxy)propanoate, HCl Salt

To a solution of 3-(2-N-Boc-aminoethoxy) propanoic acid (2.30 g, 9.82 mmol, 1.5 eq.) in dichloromethane (60 mL) cooled at 0° C. was added EDC·HCl (2.26 g, 11.79 mmol, 1.8 eq.) and dichloromethane (10 mL). The mixture was stirred at 0° C. for 5 minutes before adding DMAP (320 mg, 2.62 mmol, 0.4 eq.), pure (E)-capsaicin (2.00 g, 6.55 mmol, 1.0 eq.) and dichloromethane (6 mL). The reaction mixture was stirred at room temperature for 16 hours under a nitrogen atmosphere. Then the solvents are evaporated under reduced pressure and the crude was taken up in a mixture of EtOAc/Et₂O and water. The organic layer was successively washed with 0.5 M NaOH, brine, 0.5 M HCl & brine, dried over Na₂SO₄, filtered through a silica gel pad and evaporated under reduced pressure to afford the Boc-protected derivative as an oil. The product was directly used in the next step without further purification. The oil was taken up in dichloromethane (17 mL) and EtOAc (17 mL), the solution cooled down to 0° C. before adding 4M HCl in CPME (22 mL, 88 mmol) under nitrogen. The reaction mixture was stirred at room temperature for 6 hours, still under an inert atmosphere. The precipitate that formed was filtered, washed with a small amount of dichloromethane and triturated with Et₂O to afford the titled compound (2.13 g, 71%) as an off-white solid. 1H NMR (DMSO-d₆, 500 MHZ): δ (ppm): 0.92 (d, J=6.7 Hz, 6H), 1.30 (m, 2H), 1.51 (m, 2H), 1.93 (q, J=6.7 Hz, 2H), 2.15 (t, J=7.4 Hz, 2H), 2.21 (m, 1H), 2.85 (t, J=6.3 Hz, 2H), 2.97 (m, 2H), 3.63 (t, J=5.4 Hz, 2H), 3.73 (s, 3H), 3.76 (t, J=6.2 Hz, 2H), 4.24 (d, J=5.4 Hz, 2H), 5.27-5.39 (m, 2H), 6.81 (dd, J=8.1 Hz, J=1.6 Hz, 1H), 6.99 (bs, 1H), 7.01 (d, J=8.1 Hz, 1H), 8.01 (bs, 3H), 8.39 (m, 1H)

### Example 7

### Synthesis of (E)-2-methoxy-4-[(8-methylnon-6-enamido)methyl]phenyl 3-aminopropanoate-introduced sodium hyaluronate

Sodium hyaluronate with an average molecular weight of 750 kDa (250 mg, 2.5 eq. disaccharide unit) in a mixture of water (30 mL) and dioxane (30 mL) was stirred at room temperature for 1 hour until fully soluble. A solution of N-hydroxysuccinimide (58 mg, 0.50 mmol, 2.0 eq.) in water (2 mL), then a solution of compound obtained in example 2 (104 mg, 0.25 mmol, 1.0 eq.) in water/dioxane (1 mL/3 mL), and finally a solution of EDC (44 μL, 0.25 mmol, 1.0 eq.) in water (2 mL) were successfully added in that order with 5 minutes stirring between each addition. The reaction mixture was stirred at room temperature for 19 hours before the addition of NaHCO₃ (200 mg) as a solution in water (4 mL). The reaction mixture was stirred again at room temperature for an additional 5 hours before the successive addition of AcOH (60 μL) in water (3 mL) then NaCl (1.5 g). The reaction mixture was stirred at room temperature for 1 hour and poured into 250 mL of EtOH. The mixture was stirred at room temperature for 15 minutes and filtered. The precipitate was washed with ethanol, twice with 85% ethanol in water, twice with ethanol and several times with Et₂O to afford the titled compound as a white meringue (233 mg after overnight drying). The content of capsaicin was in the 6-10% weight/weight range as determined by ultraviolet spectroscopy.

### Example 8

### Synthesis of (E)-2-methoxy-4-[(8-methylnon-6-enamido)methyl]phenyl 3-aminopropanoate-introduced sodium hyaluronate

Sodium hyaluronate with an average molecular weight of 750 kDa (250 mg, 2.5 eq. disaccharide unit) in a mixture of water (30 mL) and dioxane (30 mL) was stirred at room temperature for 1 hour until fully soluble. A solution of N-Hydroxysulfosuccinimide (109 mg, 0.50 mmol, 2.0 eq.) in water (3 mL), then a solution of compound obtained in example 2 (104 mg, 0.25 mmol, 1.0 eq.) in water/dioxane (1 mL/3 mL), and finally a solution of EDC·HCl (48 mg, 0.25 mmol, 1.0 eq.) in water (2 mL) were successfully added in that order with 5 minutes stirring between each addition. The reaction mixture was stirred at room temperature for 19 hours before the addition of NaHCO₃ (200 mg) as a solution in water (4 mL). The reaction mixture was stirred again at room temperature for an additional 5 hours before the successive addition of AcOH (60 μL) in water (3 mL) then NaCl (1.5 g). The reaction mixture was stirred at room temperature for 1 hour and poured into 250 mL of EtOH. The mixture was stirred at room temperature for 15 minutes and filtered. The precipitate was washed with ethanol, twice with 85% ethanol in water, twice with ethanol and several times with Et₂O to afford the titled compound as a white meringue (233 mg after overnight drying). The content of capsaicin was in the 6-10% weight/weight range as determined by ultraviolet spectroscopy.

### Example 9

### Synthesis of (E)-2-methoxy-4-[(8-methylnon-6-enamido)methyl]phenyl 3-(2-aminoethoxy)propanoate-Introduced sodium hyaluronate

Sodium hyaluronate with an average molecular weight of 750 kDa (250 mg, 2.5 eq. disaccharide unit) in a mixture of water (30 mL) and dioxane (30 mL) was stirred at room temperature for 1 hour until fully soluble. A solution of N-hydroxysuccinimide (58 mg, 0.50 mmol, 2.0 eq.) in water (2 mL), then a solution of compound obtained in example 6 (115 mg, 0.25 mmol, 1.0 eq.) in water/dioxane (1 mL/3 mL), and finally a solution of EDC·HCl (48 mg, 0.25 mmol, 1.0 eq.) in water (2 mL) were successfully added in that order with 5 minutes stirring between each addition. The reaction mixture was stirred at room temperature for 19 hours before the addition of NaHCO₃ (200 mg) as a solution in water (4 mL). The reaction mixture was stirred again at room temperature for an additional 6 hours before the successive addition of AcOH (60 μL) in water (3 mL) then NaCl (1.5 g). The reaction mixture was stirred at room temperature for 2 hours and poured into 250 mL of EtOH. The mixture was stirred at room temperature for 15 minutes and filtered. The precipitate was washed with ethanol, twice with 85% ethanol in water, twice with ethanol and several times with Et₂O to afford the titled compound as a white meringue (200 mg after overnight drying). The content of capsaicin was in the 1-5% weight/weight range as determined by ultraviolet spectroscopy.

### Example 10

### Synthesis of (E)-2-methoxy-4-[(8-methylnon-6-enamido)methyl]phenyl 3-aminopropanoate-introduced sodium hyaluronate

Sodium hyaluronate with an average molecular weight of 750 kDa (250 mg, 1.25 eq. disaccharide unit) in water (25 mL) was stirred at room temperature for 1 hour until fully soluble. A solution of compound obtained in example 2 (206 mg, 0.50 mmol, 1.0 eq.) in water (10 mL) and a solution of DMTMM chloride (138 mg, 0.50 mmol, 1.0 eq.) in water (4 mL) were successfully added in that order before adjusting the pH to 6-7 with NaOH 1M. Dioxane (8 mL) was added, and the reaction mixture was stirred at room temperature for 65 hours. The slurry was poured into 250 mL of EtOH. The resulting precipitate was filtered, successively washed and triturated with EtOH 100%, EtOH 80% in water, EtOH 100%, acetone and Et₂O to afford the titled compound as a white solid (280 mg after overnight drying). The content of capsaicin was in the 11-15% weight/weight range as determined by ultraviolet spectroscopy.

### Example 11

### Synthesis of (E)-2-methoxy-4-[(8-methylnon-6-enamido)methyl]phenyl 3-aminopropanoate-introduced sodium hyaluronate

Sodium hyaluronate with an average molecular weight of 750 kDa (250 mg, 1.25 eq. disaccharide unit) was dissolved in 32 mL of water at room temperature for 60 minutes before the addition of 6 mL of dioxane and further stirring for 15 minutes. The pH was adjusted with NaOH 1M (2 drops) before the addition of a solution of compound obtained in example 2 (103 mg, 0.25 mmol, 0.5 eq.) in a H₂O/dioxane mixture (2 mL/2 mL). The reaction was stirred for 10 minutes, the pH adjusted again with NaOH 1M (2 drops) before adding a solution of DMTMM chloride (69 mg, 0.25 mmol, 0.5 eq.) in water (4 mL). A new addition of dioxane (2 mL) was performed before a final adjustment of the pH to 6-7 with NaOH 1M (2 drops). The resulting mixture was stirred at room temperature for 19 hours. A second portion of compound obtained in example 2 (103 mg, 0.25 mmol, 0.5 eq.) as a solution in a H₂O/dioxane mixture (2 mL/2 mL) was added, the pH adjusted with NaOH 1M (2 drops) before adding a second portion of DMTMM chloride (69 mg, 0.25 mmol, 0.5 eq.) as a solution in H₂O (4 mL). As before, a last adjustment of the pH to 6-7 was done with NaOH 1M (6 drops). The reaction was stirred for an additional 23 hours. Then, a solution of NaCl (0.5 g) in H₂O (4 mL) was added and the reaction mixture stirred for 15 minutes and poured into 250 mL of EtOH. The resulting precipitate was stirred in the EtOH/H₂O/dioxane mixture at room temperature for 15 minutes, and left for decantation for 15 minutes. The supernatant was discarded and the precipitate filtered. The solid was successively washed and triturated with EtOH 100%, EtOH 85% in water, EtOH 100%, acetone and Et₂O to afford the tithe compound as a white meringue/solid (190 mg after overnight drying). The content of capsaicin was in the 11-15% weight/weight range as determined by ultraviolet spectroscopy.

### Example 12

### Synthesis of (E)-2-methoxy-4-[(8-methylnon-6-enamido)methyl]phenyl 3-(2-aminoethoxy)propanoate-Introduced sodium hyaluronate

Sodium hyaluronate with an average molecular weight of 750 kDa (250 mg, 1.25 eq. disaccharide unit) was dissolved in 30 mL of water at room temperature for 45 minutes before the addition of 6 mL of dioxane and further stirring for 15 minutes. The pH was adjusted with NaOH 1M (2 drops) before the addition of a solution of compound obtained in example 6 (115 mg, 0.25 mmol, 0.5 eq.) in a H₂O/dioxane mixture (2 mL/2 mL). The reaction was stirred for 10 minutes, the pH adjusted again with NaOH 1M (2 drops) before adding a solution of DMTMM chloride (69 mg, 0.25 mmol, 0.5 eq.) in water (4 mL). A new addition of dioxane (2 mL) was performed before a final adjustment of the pH to 6-7 with NaOH 1M (2 drops). The resulting mixture was stirred at room temperature for 21 hours. A second portion of compound obtained in example 6 (115 mg, 0.25 mmol, 0.5 eq.) as a solution in a H₂O/dioxane mixture (2 mL/2 mL) was added, the pH adjusted with NaOH 1M (2 drops) before adding a second portion of DMTMM chloride (69 mg, 0.25 mmol, 0.5 eq.) as a solution in H₂O (4 mL). As before, a last adjustment of the pH to 6-7 was done with NaOH 1M (6 drops). The reaction was stirred for an additional 29 hours. Then, a solution of NaCl (0.75 g) in H₂O (4 mL) was added and the reaction mixture stirred for 15 minutes and poured into 250 mL of EtOH. The resulting precipitate was stirred in the EtOH/H₂O/dioxane mixture at room temperature for 15 minutes and left for decantation for 15 minutes. The supernatant was discarded, and the precipitate filtered. The solid was successively washed and triturated with EtOH 100%, EtOH 85% in water, EtOH 100%, acetone and Et₂O to afford titled compound as a white meringue (220 mg after overnight drying). The content of capsaicin was in the 6-10% weight/weight range as determined by ultraviolet spectroscopy.

### Example 13

### Synthesis of (E)-2-methoxy-4-[(8-methylnon-6-enamido)methyl]phenyl 3-aminopropanoate-introduced sodium hyaluronate

Sodium hyaluronate with an average molecular weight of 880 kDa (500 mg, 1.25 eq. disaccharide unit) was stirred in H₂O/EtOH (50 mL/50 mL) for 90 minutes. A solution of compound obtained in example 2 (206 mg, 0.5 mmol, 1.0 eq.) in a H₂O/EtOH mixture (2 mL/2 mL) was added. The reaction was stirred for 10 minutes before adding a solution of DMTMM chloride (138 mg, 0.5 mmol, 1.0 eq.) in water (4 mL). The pH was adjusted to 6-7 with NaOH 1M (10 drops) and the resulting mixture stirred at room temperature for 29 hours. Then, NaCl (1.0 g) was added. The slurry was stirred at room temperature for 15 minutes until solubilisation of the salt and poured into 500 mL of EtOH. The resulting precipitate was stirred in the EtOH/H₂O mixture at room temperature for 15 minutes, and left for decantation for 45 minutes. The supernatant was discarded and the precipitate filtered. The solid was successively washed and triturated with EtOH 100%, EtOH 85% in water, EtOH 100%, acetone and Et₂O to afford the titled compound as a white meringue (400 mg). The content of capsaicin was in the 11-15% weight/weight range determined by ultraviolet spectroscopy.

### Example 14

### Synthesis of (E)-2-methoxy-4-[(8-methylnon-6-enamido)methyl]phenyl 3-(2-aminoethoxy)propanoate-Introduced sodium hyaluronate

Sodium hyaluronate with an average molecular weight of 880 kDa (500 mg, 2.5 eq. disaccharide unit) was dissolved in 50 mL of water at room temperature for 30 minutes before the addition of 50 mL of absolute ethanol. The mixture was further stirred at room temperature for 20 minutes and a solution of compound obtained in example 6 (229 mg, 1.0 mmol, 1.0 eq.) in a H₂O/EtOH mixture (2 mL/2 mL) was added. The reaction was stirred for 10 minutes before adding a solution of DMTMM chloride (138 mg, 0.5 mmol, 1.0 eq.) in water (4 mL). The pH was adjusted to 6-7 with NaOH 1M (10 drops) and the resulting mixture stirred at room temperature for 24 hours. NaCl (1.0 g) was then added. The slurry was stirred at room temperature for 10 minutes until solubilisation of the salt and poured into 600 mL of EtOH. The resulting precipitate was stirred in the EtOH/H₂O mixture at room temperature for 15 minutes and left for decantation for 30 minutes. The supernatant was discarded and the precipitate filtered. The solid was successively washed and triturated with EtOH 100%, EtOH 85% in water, EtOH 100%, acetone and Et₂O to afford the titled compound as a white meringue (485 mg after overnight drying). The content of capsaicin was in the 6-10% weight/weight range as determined by ultraviolet spectroscopy.

### Example 15

### Synthesis of (E)-2-methoxy-4-[(8-methylnon-6-enamido)methyl]phenyl 2-(2-aminoethoxy)acetate, TFA Salt

To a solution of 5-(t-Butyloxycarbonyl-amino)-3-oxapentanoic acid dicyclohexylamine (506 mg, 1.26 mmol, 1.6 eq.) in dichloromethane (8 mL) cooled at 0° C. was added EDC·HCl (303 mg, 1.58 mmol, 2.0 eq.) as a powder. The mixture was stirred at 0° C. for 5 minutes before adding DMAP (58 mg, 0.47 mmol, 0.6 eq.) and pure (E)-capsaicin (240 mg, 0.79 mmol, 1.0 eq.) as powders. The reaction mixture was further diluted with dichloromethane (6 mL) to help solubilization and was stirred at room temperature for 20 hours under a nitrogen atmosphere. The mixture was diluted with EtOAc and the insoluble filtered. The organic solution was successively washed with water, 0.5 M NaOH, brine, 0.5 M HCl & brine, dried over Na₂SO₄, filtered through a silica gel pad and evaporated under reduced pressure to afford the Boc-protected derivative as a yellow oil. The product was directly used in the next step without further purification. The oil was taken up in dichloromethane (10 mL) and the solution cooled down to 0° C. before adding TFA (0.9 mL, 11.8 mmol). The reaction mixture was stirred at room temperature for 14 hours under a nitrogen atmosphere. The mixture was diluted with methanol, filtered and evaporated. The oil was taken up in methanol and evaporated two additional times to afford the titled compound (440 mg, quantitative) as an orange oil after further drying under high vacuum. 1H NMR (DMSO-d₆, 500 MHz): δ (ppm): 0.92 (d, J=6.7 Hz, 6H), 1.30 (m, 2H), 1.51 (m, 2H), 1.95 (q, J=6.6 Hz, 2H), 2.14 (t, J=7.3 Hz, 2H), 2.20 (m, 1H), 3.04 (m, 2H), 3.75 (s, 3H), 4.25 (d, J=5.9 Hz, 2H), 4.48 (s, 2H), 5.28-5.40 (m, 2H), 6.83 (dd, J=8.1 Hz, J=1.7 Hz, 1H), 7.02 (d, J=1.7 Hz, 1H), 7.05 (d, J=8.1 Hz, 1H), 7.82 (bs, 3H), 8.34 (m, 1H)

### Example 16

### Synthesis of (E)-2-methoxy-4-[(8-methylnon-6-enamido)methyl]phenyl 3-(2-aminoethoxy)propanoate-Introduced sodium hyaluronate

Sodium hyaluronate with an average molecular weight of 880 kDa (250 mg, 1.6 eq. disaccharide unit) was dissolved in 25 mL of water at room temperature for 50 minutes before the addition of 25 mL of absolute ethanol. The mixture was further stirred at room temperature for 40 minutes before a first pH adjustment with the addition of 2 drops of NaOH 1M. A solution of compound obtained in example 6 (172 mg, 0.38 mmol, 1.0 eq.) in H₂O/EtOH (2 mL/2 mL) was added and the reaction was stirred for 5 minutes before a second pH adjustment with the addition of 2 drops of NaOH 1M. Then, a solution of DMTMM chloride (104 mg, 0.38 mmol, 1.0 eq.) in H₂O (4 mL) was added and the pH was again adjusted to 6-7 with 6 drops of NaOH 1M. The reaction was stirred at room temperature for 29 hours. NaCl (0.5 g) was then added. The slurry was stirred at room temperature for 30 minutes until solubilisation of the salt and poured into 300 mL of EtOH. The resulting precipitate was stirred in the EtOH/H₂O mixture at room temperature for 15 minutes, and left for decantation for 40 minutes. The supernatant was discarded and the precipitate filtered. The solid was successively washed and triturated with EtOH 100%, EtOH 85% in water, EtOH 100%, acetone and Et₂O to afford the titled compound as a white meringue (205 mg after overnight drying). The content of capsaicin was in the 6-10% weight/weight range as determined by ultraviolet spectroscopy.

### Example 17

### Synthesis of (E)-2-methoxy-4-[(8-methylnon-6-enamido)methyl]phenyl (5-aminopentyl)carbamate, HCl Salt

To a solution of pure (E)-capsaicin (400 mg, 1.31 mmol, 1.00 eq.) in dichloromethane (20 mL) was added 4-nitrophenyl chloroformate (280 mg, 1.38 mmol, 1.05 eq.) then triethylamine (540 μL, 3.93 mmol, 3.0 eq.) at 0° C. and under a nitrogen atmosphere. The mixture was stirred at room temperature for 6 hours before adding 1-hydroxybenzotriazole hydrate (220 mg, 1.44 mmol, 1.10 eq.) as a powder, dichloromethane (1 mL) then a solution of N-Boc-cadaverine (318 mg, 1.57 mmol, 1.20 eq.) in dichloromethane (3 mL). The reaction mixture was stirred at room temperature for 21 hours, quenched with 1 M HCl, and successively washed with 1 M HCl (×2), a saturated sodium bicarbonate solution (×4) and brine (×2). The organic layer was dried over Na₂SO₄, filtered through a silica gel pad. Further elution was carried out with dichloromethane, diethyl ether then ethyl acetate. The ethyl acetate fraction was collected and evaporated to afford a yellow solid. The solid was taken up in diethyl ether and the mixture was stirred overnight before filtration. The oily residue is solubilized in dichloromethane and evaporated to afford the pure Boc-protected derivative. The product was directly used in the next step and taken up in dichloromethane (15 mL) before adding 2M HCl in Et₂O (15 mL, 30 mmol) under nitrogen and at 0° C. The reaction mixture was stirred at room temperature for 8 hours, still under an inert atmosphere. The precipitate that formed was filtered, washed with a small amount of dichloromethane and triturated with Et₂O, acetone and again Et₂O several times. The residue was resuspended in diethyl ether and left overnight at room temperature without stirring. The supernatant was removed and the solid further dried under reduced pressure to afford the titled compound (370 mg, 74%) as a white solid. 1H NMR (DMSO-d₆, 500 MHz): δ (ppm): 0.93 (d, J=6.6 Hz, 6H), 1.24-1.37 (m, 4H), 1.42-1.58 (m, 6H), 1.94 (q, J=6.6 Hz, 2H), 2.13 (t, J=7.3 Hz, 2H), 2.21 (m, 1H), 2.76 (m, 2H), 3.02 (q, J=6.6 Hz, 2H), 3.72 (s, 3H), 4.23 (d, J=6.0 Hz, 2H), 5.28-5.40 (m, 2H), 6.77 (dd, J=8.0 Hz, J=1.8 Hz, 1H), 6.94 (bs, 1H), 6.96 (d, J=8.0 Hz, 1H), 7.66 (m, 1H), 7.74 (bs, 3H), 8.33 (m, 1H)

### Example 18

### Synthesis of (E)-2-methoxy-4-[(8-methylnon-6-enamido)methyl]phenyl [2-(2-aminoethoxy)ethyl]carbamate, HCl Salt

To a solution of pure (E)-capsaicin (400 mg, 1.31 mmol, 1.00 eq.) in dichloromethane (20 mL) was added 4-nitrophenyl chloroformate (280 mg, 1.38 mmol, 1.05 eq.) then triethylamine (540 μL, 3.93 mmol, 3.0 eq.) at 0° C. and under a nitrogen atmosphere. The mixture was stirred at room temperature for 6 hours before adding 1-hydroxybenzotriazole hydrate (220 mg, 1.44 mmol, 1.10 eq.) as a powder, dichloromethane (1 mL) then a solution of N-Boc-2-(2-aminoethoxy)ethylamine (321 mg, 1.57 mmol, 1.20 eq.) in dichloromethane (3 mL). The reaction mixture was stirred at room temperature for 21 hours, quenched with 1 M HCl, and successively washed with 1 M HCl (×2), a saturated sodium bicarbonate solution (×4) and brine (×2). The organic layer was dried over Na₂SO₄, filtered through a silica gel pad. Further elution was carried out with dichloromethane, diethyl ether then ethyl acetate. The ethyl acetate fraction was collected and evaporated to afford the Boc-protected derivative as a colorless oil or a white solid. The product was directly used in the next step without further purification and taken up in dichloromethane (10 mL) before adding 2M HCl in Et₂O (15 mL, 30 mmol) under nitrogen and at 0° C. The reaction mixture was stirred at room temperature for 6 hours, still under an inert atmosphere. The precipitate that formed was filtered, washed with a small amount of dichloromethane and triturated with Et₂O, acetone and again Et₂O to afford a sticky solid. The residue was resuspended in diethyl ether and left overnight at room temperature without stirring. The supernatant was removed and the solid further dried under reduced pressure to afford the titled compound (456 mg, 74%) as a white solid. 1H NMR (DMSO-d₆, 500 MHz): δ (ppm): 0.93 (d, J=6.6 Hz, 6H), 1.30 (m, 2H), 1.51 (m, 2H), 1.94 (q, J=6.6 Hz, 2H), 2.13 (t, J=7.3 Hz, 2H), 2.21 (m, 1H), 2.98 (m, 2H), 3.24 (m, 2H), 3.50 (t, J=5.5 Hz, 2H), 3.61 (t, J=5.5 Hz, 2H), 3.73 (s, 3H), 4.23 (d, J=6.0 Hz, 2H), 5.28-5.40 (m, 2H), 6.77 (dd, J=8.0 Hz, J=1.6 Hz, 1H), 6.96 (d, J=1.6 Hz, 1H), 6.98 (d, J=8.0 Hz, 1H), 7.75 (m, 1H), 8.02 (bs, 3H), 8.36 (m, 1H)

### Example 19

### Synthesis of (E)-2-methoxy-4-[(8-methylnon-6-enamido)methyl]phenyl 3-(2-aminoethoxy)propanoate-Introduced sodium hyaluronate

Sodium hyaluronate with an average molecular weight of 880 kDa (250 mg, 1.6 eq. disaccharide unit) was dissolved in 25 mL of water at room temperature for 30 minutes before the addition of 25 mL of absolute ethanol. The mixture was further stirred at room temperature for 15 minutes before a first pH adjustment with the addition of 2 drops of NaOH 1M. A solution of compound obtained in example 6 (172 mg, 0.38 mmol, 1.0 eq.) in H₂O/EtOH (2 mL/2 mL) was added and the reaction was stirred for 5 minutes before a second pH adjustment with the addition of 2 drops of NaOH 1M. Then, a solution of DMTMM tetrafluoroborate (128 mg, 0.38 mmol, 1.0 eq.) in acetonitrile (4 mL) was added and the pH was again adjusted to 6-7 with 6 drops of NaOH 1M. The reaction was stirred at room temperature for 29 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation for 45 minutes. The supernatant was discarded and the precipitate filtered. The solid was successively washed and triturated with EtOH 100%, EtOH 85% in water, EtOH 100%, acetone and Et₂O to afford the titled compound as a white meringue (215 mg after overnight drying). The content of capsaicin was in the 16-20% weight/weight range as determined by ultraviolet spectroscopy.

### Example 20

### Synthesis of (E)-2-methoxy-4-[(8-methylnon-6-enamido)methyl]phenyl [2-(2-aminoethoxy)ethyl]carbamate-Introduced sodium hyaluronate

Sodium hyaluronate with an average molecular weight of 880 kDa (250 mg, 1.6 eq. disaccharide unit) was dissolved in 25 mL of water at room temperature for 20 minutes before the addition of 25 mL of absolute ethanol. The mixture was further stirred at room temperature for 10 minutes before a first pH adjustment with the addition of 2 drops of NaOH 1M. A solution of compound obtained in example 18 (179 mg, 0.38 mmol, 1.0 eq.) in H₂O/EtOH (2 mL/2 mL) was added and the reaction was stirred for 5 minutes before a second pH adjustment with the addition of 2 drops of NaOH 1M. Then, a solution of DMTMM chloride (104 mg, 0.38 mmol, 1.0 eq.) in H₂O (4 mL) was added and the pH was again adjusted to 6-7 with 4 drops of NaOH 1M. The reaction was stirred at room temperature for 29 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 10 minutes, and left for decantation for 15 minutes. The supernatant was discarded and the precipitate filtered. The solid was successively washed and triturated with EtOH 100%, EtOH 85% in water, EtOH 100%, acetone and Et₂O to afford the titled compound as a white meringue/solid (195 mg after overnight drying). The content of capsaicin was in the 11-15% weight/weight range as determined by ultraviolet spectroscopy.

### Example 21

### Synthesis of (E)-2-methoxy-4-[(8-methylnon-6-enamido)methyl]phenyl (5-aminopentyl)carbamate-Introduced sodium hyaluronate

Sodium hyaluronate with an average molecular weight of 880 kDa (250 mg, 1.6 eq. disaccharide unit) was dissolved in 25 mL of water at room temperature for 20 minutes before the addition of 25 mL of absolute ethanol. The mixture was further stirred at room temperature for 10 minutes before a first pH adjustment with the addition of 2 drops of NaOH 1M. A solution of compound obtained in example 17 (179 mg, 0.38 mmol, 1.0 eq.) in H₂O/EtOH (2 mL/2 mL) was added and the reaction was stirred for 5 minutes before a second pH adjustment with the addition of 2 drops of NaOH 1M. Then, a solution of DMTMM chloride (104 mg, 0.38 mmol, 1.0 eq.) in H₂O (4 mL) was added and the pH was again adjusted to 6-7 with 2 drops of NaOH 1M. The reaction was stirred at room temperature for 29 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 10 minutes, and left for decantation for 15 minutes. The supernatant was discarded and the precipitate filtered. The solid was successively washed and triturated with EtOH 100%, EtOH 85% in water, EtOH 100%, acetone and Et₂O to afford the titled compound as a white meringue/solid (230 mg after overnight drying). The content of capsaicin was in the 11-15% weight/weight range determined by ultraviolet spectroscopy.

### Example 22

### Synthesis of 2-methoxy-4-{[(E)-8-methylnon-6-enamido]methyl]phenyl trans-4-aminocyclohexane-1-carboxylate, HCl Salt

To a solution of trans-4-(tert-butoxycarbonylamino)cyclohexanecarboxylic acid (3.2 g, 13.10 mmol, 2.0 eq.) in dichloromethane (80 mL) cooled at 0° C. was added EDC·HCl (3.77 g, 19.65 mmol, 3.0 eq.), dichloromethane (4 mL) then N-methylimidazole (520 μL, 6.55 mmol, 1.0 eq.). The mixture was stirred at 0° C. for 5 minutes before adding pure (E)-capsaicin (2.0 g, 6.55 mmol, 1.0 eq.) and dichloromethane (4 mL). The reaction mixture was stirred at room temperature for 22 hours under a nitrogen atmosphere. Then the solvents are evaporated under reduced pressure (water bath at 30° C.) and the crude was taken up in a mixture of EtOAc/Et₂O/DCM and water. The organic layer was successively washed with water, 0.5 M NaOH, 0.5 M HCl & brine, dried over Na₂SO₄, filtered through a silica gel pad, eluted with EtOAc/DCM and evaporated under reduced pressure to afford the Boc-protected derivative as a solid. The product was directly used in the next step without further purification. The solid was taken up in dichloromethane (30 mL) and EtOAc (20 mL), the solution cooled down to 0° C. before adding 4M HCl in CPME (30 mL, 120 mmol) under nitrogen. The reaction mixture was stirred at room temperature for 8 hours, still under an inert atmosphere. The precipitate that formed was filtered, washed with a small amount of dichloromethane and triturated with Et₂O. The residue was resuspended in diethyl ether and left overnight at room temperature without stirring. The supernatant was removed, the solid filtered and washed with diethyl ether to afford the titled compound (3.0 g, 98%) as a white solid. 1H NMR (DMSO-d₆, 500 MHz): δ (ppm): 0.92 (d, J=6.7 Hz, 6H), 1.29 (m, 3H), 1.41 (m, 2H), 1.49-1.57 (m, 4H), 1.93 (m, 3H), 2.02 (bd, 2H), 2.10 (bd, 2H), 2.13 (t, J=7.3 Hz, 2H), 2.20 (m, 1H), 2.53 (m, 1H), 3.03 (m, 1H), 3.72 (s, 3H), 4.24 (d, J=5.9 Hz, 2H), 5.28-5.39 (m, 2H), 6.80 (dd, J=8.1 Hz, J=1.2 Hz, 1H), 6.97 (d, J=8.1 Hz, 1H), 6.98 (d, J=1.2 Hz, 1H), 8.03 (bs, 3H), 8.33 (m, 1H)

### Example 23

### Synthesis of (E)-2-methoxy-4-[(8-methylnon-6-enamido)methyl]phenyl 3-aminopyrrolidine-1-carboxylate, HCl Salt

To a solution of pure (E)-capsaicin (800 mg, 2.62 mmol, 1.0 eq.) in dichloromethane (80 mL) was added bis(4-nitrophenyl) carbonate (876 mg, 2.88 mmol, 1.1 eq.) then triethylamine (1.09 mL, 7.86 mmol, 3.0 eq.) at 0° C. and under a nitrogen atmosphere. The mixture was stirred at room temperature for 5 hours before adding 1-hydroxybenzotriazole hydrate (481 mg, 3.14 mmol, 1.2 eq.) as a powder, dichloromethane (4 mL) then 3-(tert-Butoxycarbonylamino) pyrrolidine (635 mg, 3.41 mmol, 1.3 eq.) also as a powder, and again dichloromethane (2 mL). The reaction mixture was stirred at room temperature for 23 hours, quenched with 1 M HCl, and successively washed with 1 M HCl (×2), a saturated sodium bicarbonate solution (×4) and brine (×2). The organic layer was dried over Na₂SO₄, filtered through a silica gel pad. Further elution was carried out with dichloromethane, diethyl ether then ethyl acetate. The ethyl acetate fraction was collected and evaporated to afford the Boc-protected derivative. The product was directly used in the next step without further purification and taken up in dichloromethane (10 mL) before adding 2M HCl in Et₂O (20 mL, 40 mmol) under nitrogen and at 0° C. The reaction mixture was stirred at room temperature for 6 hours, still under an inert atmosphere. The heterogeneous mixture was degassed and partially evaporated under reduced pressure. The residue was triturated with Et₂O and the mixture stirred at room temperature overnight. The supernatant was discarded, and the residue taken up and further triturated with Et₂O. The mixture was evaporated and dried under reduced pressure to afford the titled compound (1.1 g, 81%) as a white solid. 1H NMR (DMSO-d₆, 500 MHz): δ (ppm): 0.93 (d, J=6.6 Hz, 6H), 1.30 (m, 2H), 1.52 (m, 2H), 1.94 (q, J=6.6 Hz, 2H), 1.97-2.07 (m, 1H), 2.14 (t, J=7.3 Hz, 2H), 2.21 (m, 1H), 2.22-2.31 (m, 1H), 3.50-3.70 (m, 2H), 3.74 (s, 3H), 3.77 (m, 1H), 3.86 (m, 1H), 4.24 (d, J=5.9 Hz, 2H), 5.28-5.39 (m, 2H), 6.79 (dd, J=8.0 Hz, 1H), 6.96 (bs, 1H), 6.99 (d, J=8.0 Hz, 1H), 7.75 (m, 1H), 8.27 (bs, 3H), 8.32 (m, 1H)

### Example 24

### Synthesis of (E)-2-methoxy-4-[(8-methylnon-6-enamido)methyl]phenyl 3-aminopyrrolidine-1-carboxylate-Introduced sodium hyaluronate

Sodium hyaluronate with an average molecular weight of 880 kDa (250 mg, 1.6 eq. disaccharide unit) was dissolved in 25 mL of water at room temperature for 20 minutes before the addition of 25 mL of absolute ethanol. The mixture was further stirred at room temperature for 5 minutes before a first pH adjustment with the addition of 2 drops of NaOH 1M. A solution of compound obtained in example 23 (197 mg, 0.38 mmol, 1.0 eq.) in H₂O/EtOH (2 mL/2 mL) was added and the reaction was stirred for 5 minutes before a second pH adjustment with the addition of 2 drops of NaOH 1M. Then, a solution of DMTMM chloride (104 mg, 0.38 mmol, 1.0 eq.) in H₂O (4 mL) was added and the pH was again adjusted to 6-7 with 4 drops of NaOH 1M. The reaction was stirred at room temperature for 50 hours. NaCl (0.5 g) was then added. The slurry was stirred at room temperature for 20 minutes until solubilisation of the salt and poured into 300 mL of EtOH. The resulting precipitate was stirred in the EtOH/H₂O mixture at room temperature for 20 minutes, and left for decantation for 60 minutes. The supernatant was discarded and the precipitate filtered. The solid was successively washed and triturated with EtOH 100%, EtOH 85% in water, EtOH 100%, acetone and Et₂O to afford the titled compound as a white solid (260 mg after overnight drying). The content of capsaicin was in the 16-20% weight/weight range as determined by ultraviolet spectroscopy.

### Example 25

### Synthesis of 2-methoxy-4-{[(E)-8-methylnon-6-enamido]methyl]phenyl trans-4-aminocyclohexane-1-carboxylate-Introduced sodium hyaluronate

Sodium hyaluronate with an average molecular weight of 880 kDa (250 mg, 1.4 eq. disaccharide unit) was dissolved in 27 mL of water at room temperature for 30 minutes before the addition of 25 mL of absolute ethanol. The mixture was further stirred at room temperature for 15 minutes before a first pH adjustment with the addition of 2 drops of NaOH 1M. A solution of compound obtained in example 22 (207 mg, 0.44 mmol, 1.0 eq.) in H₂O/EtOH (2 mL/2 mL) was added and the reaction was stirred for 5 minutes before a second pH adjustment with the addition of 2 drops of NaOH 1M. Then, a solution of DMTMM chloride (123 mg, 0.44 mmol, 1.0 eq.) in H₂O (4 mL) was added and the pH was again adjusted to 6-7 with 6 drops of NaOH 1M. The reaction was stirred at room temperature for 46 hours. NaCl (0.5 g) was then added. The slurry was stirred at room temperature for 25 minutes until solubilisation of the salt and poured into 300 mL of EtOH. The resulting precipitate was stirred in the EtOH/H₂O mixture at room temperature for 25 minutes, and left for decantation for 30 minutes. The supernatant was discarded and the precipitate filtered. The solid was successively washed and triturated with EtOH 100%, EtOH 85% in water, EtOH 100%, acetone and Et₂O to afford the titled compound as a white meringue (180 mg after overnight drying). The content of capsaicin was in the 1-5% weight/weight range as determined by ultraviolet spectroscopy.

### Example 26

### 2-methoxy-4-{[(E)-8-methylnon-6-enamido]methyl]phenyl trans-4-aminocyclohexane-1-carboxylate-Introduced sodium hyaluronate

Sodium hyaluronate with an average molecular weight of 880 kDa (250 mg, 1.4 eq. disaccharide unit) was dissolved in 27 mL of water at room temperature for 30 minutes before the addition of 25 mL of absolute ethanol. The mixture was further stirred at room temperature for 15 minutes before a first pH adjustment with the addition of 2 drops of NaOH 1M. A solution of obtained in example 22 (207 mg, 0.44 mmol, 1.0 eq.) in H₂O/EtOH (2 mL/2 mL) was added and the reaction was stirred for 5 minutes before a second pH adjustment with the addition of 2 drops of NaOH 1M. Then, a solution of DMTMM tetrafluoroborate (145 mg, 0.44 mmol, 1.0 eq.) in H₂O/acetonitrile (1 mL/3 mL) was added and the pH was again adjusted to 6-7 with 6 drops of NaOH 1M. The reaction was stirred at room temperature for 46 hours. NaCl (0.5 g) was then added. The slurry was stirred at room temperature for 25 minutes until solubilisation of the salt and poured into 300 mL of EtOH. The resulting precipitate was stirred in the EtOH/H₂O mixture at room temperature for 25 minutes, and left for decantation for 30 minutes. The supernatant was discarded and the precipitate filtered. The solid was successively washed and triturated with EtOH 100%, EtOH 85% in water, EtOH 100%, acetone and Et₂O to afford the titled compound as a white meringue (230 mg after overnight drying). The content of capsaicin was in the 1-5% weight/weight range as determined by ultraviolet spectroscopy.

### Example 27

### Synthesis of (E)-2-methoxy-4-[(8-methylnon-6-enamido)methyl]phenyl (2-aminoethyl)(isopropyl)carbamate, HCl Salt

To a solution of pure (E)-capsaicin (1.2 g, 3.93 mmol, 1.0 eq.) in dichloromethane (100 mL) was added bis(4-nitrophenyl) carbonate (1.31 g, 4.32 mmol, 1.1 eq.) then triethylamine (1.64 mL, 11.79 mmol, 3.0 eq.) at 0° C. and under a nitrogen atmosphere. The mixture was stirred at room temperature for 6 hours before adding 2-Pyridinol 1-oxide (HOPO, 524 mg, 4.72 mmol, 1.2 eq.) as a powder, dichloromethane (4 mL) then N′-Boc-N-isopropylethylenediamine (1.04 g, 5.12 mmol, 1.3 eq.) also as a powder. The reaction mixture was stirred at room temperature for 21 hours, quenched with 1 M HCl, and successively washed with 1 M HCl (×2), a saturated sodium bicarbonate solution (×4) and brine (×2). The organic layer was dried over Na₂SO₄, filtered through a silica gel pad. Further elution was carried out with dichloromethane, diethyl ether then ethyl acetate. The ether and ethyl acetate fractions were collected and evaporated to afford the Boc-protected derivative. The product was directly used in the next step without further purification and taken up in dichloromethane (15 mL) before adding 2M HCl in Et₂O (30 mL, 60 mmol) under nitrogen and at 0° C. The reaction mixture was stirred at room temperature for 5 hours, still under an inert atmosphere. The heterogeneous mixture was degassed and partially evaporated under reduced pressure. The residue was triturated with Et₂O, and the mixture stirred at room temperature overnight. The supernatant was discarded, and the residue taken up and further triturated with Et₂O. The mixture was evaporated and dried under reduced pressure to afford the titled compound (1.3 g, 71%) as a white solid. 1H NMR (DMSO-d₆, 500 MHZ): δ (ppm): 0.93 (d, J=6.6 Hz, 6H), 1.15-1.23 (m, 6H), 1.30 (m, 2H), 1.52 (m, 2H), 1.94 (q, J=6.6 Hz, 2H), 2.13 (t, J=7.3 Hz, 2H), 2.21 (m, 1H), 2.97 (m, 2H), 3.46 (m, 2H), 3.73 (s, 3H), 4.21 (m, 1H), 4.24 (d, J=5.9 Hz, 2H), 5.29-5.39 (m, 2H), 6.79 (dd, J=8.1 Hz, J=1.5 Hz, 1H), 6.97 (d, J=1.5 Hz, 1H), 7.04 (d, J=8.1 Hz, 1H), 7.75 (m, 1H), 7.98 (bs, 3H), 8.33 (m, 1H)

### Example 28

### Synthesis of 2-methoxy-4-{[(E)-8-methylnon-6-enamido]methyl}phenyl L-isoleucinate, HCl Salt

To a solution of N-(tert-Butoxycarbonyl) isoleucine (3.0 g, 13.10 mmol, 2.0 eq.) in dichloromethane (80 mL) cooled at 0° C. was added EDC·HCl (3.77 g, 19.65 mmol, 3.0 eq.) then N-methylimidazole (520 μL, 6.55 mmol, 1.0 eq.). The mixture was stirred at 0° C. for 10 minutes before adding pure (E)-capsaicin (2.0 g, 6.55 mmol, 1.0 eq.) and dichloromethane (4 mL). The reaction mixture was stirred at room temperature for 22 hours under a nitrogen atmosphere. Then the solvents are evaporated under reduced pressure (water bath at 30° C.) and the crude was taken up in a mixture of EtOAc/Et₂O and water. The organic layer was successively washed with water, 0.5 M NaOH, 0.5 M HCl & brine, dried over Na₂SO₄, filtered through a silica gel pad, eluted with Et₂O and evaporated under reduced pressure to afford the Boc-protected derivative as an oil. The product was directly used in the next step without further purification. The oil was taken up in dichloromethane (12 mL) and EtOAc (12 mL), the solution cooled down to 0° C. before adding 4M HCl in CPME (25 mL, 100 mmol) under nitrogen. The reaction mixture was stirred at room temperature for 6 hours, still under an inert atmosphere. The heterogeneous mixture was degassed and partially evaporated under reduced pressure. The supernatant was discarded and the residue triturated with Et₂O. The mixture was stirred at room temperature for 2 hours and left overnight without agitation. The ether was removed under reduced pressure to afford the titled compound (2.10 g, 70%) as a white solid. 1H NMR (DMSO-d₆, 500 MHz): δ (ppm): 0.92 (d, J=6.7 Hz, 6H), 0.96 (m, 3H), 1.07 (d, J=6.8 Hz, 3H), 1.30 (m, 2H), 1.40 (m, 1H), 1.53 (m, 2H), 1.63 (m, 1H), 1.94 (q, J=6.7 Hz, 2H), 2.07 (m, 1H), 2.15 (t, J=7.4 Hz, 2H), 2.20 (m, 1H), 3.75 (s, 3H), 4.26 (d, J=5.4 Hz, 2H), 5.28-5.39 (m, 2H), 6.86 (dd, J=8.3 Hz, J=1.2 Hz, 1H), 7.05 (bs, 1H), 7.06 (d, J=8.3 Hz, 1H), 8.38 (m, 1H), 8.66 (bs, 3H)

### Example 29

### Synthesis of 2-methoxy-4-{[(E)-8-methylnon-6-enamido]methyl}phenyl L-isoleucinate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 20 minutes before a first pH adjustment with the addition of 1 drop of NaOH 1M. A solution of compound obtained in example 28 (114 mg, 0.25 mmol, 1.0 eq.) in H₂O/EtOH (2 mL/2 mL) was added and the reaction was stirred for 5 minutes before adding a solution of DMTMM chloride (69 mg, 0.25 mmol, 1.0 eq.) in H₂O (4 mL). The pH was again adjusted to 6-7 with 4 drops of NaOH 1M. The reaction was stirred at room temperature for 44 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation for 15 minutes. The supernatant was discarded and the precipitate filtered. The solid was successively washed and triturated with EtOH 100%, EtOH 85% in water, EtOH 100%, acetone and Et₂O to afford the titled compound as a white meringue (200 mg after overnight drying). The content of capsaicin was in the 11-15% weight/weight range as determined by ultraviolet spectroscopy.

### Example 30

### Synthesis of (E)-2-methoxy-4-[(8-methylnon-6-enamido)methyl]phenyl (2-aminoethyl)(isopropyl)carbamate-Introduced sodium hyaluronate

Sodium hyaluronate with an average molecular weight of 880 kDa (250 mg, 1.6 eq. disaccharide unit) was dissolved in 25 mL of water at room temperature for 20 minutes before the addition of 25 mL of absolute ethanol. The mixture was further stirred at room temperature for 15 minutes before a first pH adjustment with the addition of 2 drops of NaOH 1M. A solution of compound obtained in example 27 (179 mg, 0.38 mmol, 1.0 eq.) in H₂O/EtOH (2 mL/2 mL) was added and the reaction was stirred for 5 minutes before a second pH adjustment with the addition of 2 drops of NaOH 1M. Then, a solution of DMTMM chloride (104 mg, 0.38 mmol, 1.0 eq.) in H₂O (4 mL) was added and the pH was again adjusted to 6-7 with 2 drops of NaOH 1M. The reaction was stirred at room temperature for 48 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation for 15 minutes. The supernatant was discarded and the precipitate filtered. The solid was successively washed and triturated with EtOH 100%, EtOH 85% in water, EtOH 100%, acetone and Et₂O to afford the titled compound as a white solid (210 mg after overnight drying). The content of capsaicin was in the 21-25% weight/weight range as determined by ultraviolet spectroscopy.

### Example 31

### Synthesis of 2-methoxy-4-{[(E)-8-methylnon-6-enamido]methyl}phenyl trans-4-(2-aminoacetamido)cyclohexane-1-carboxylate, HCl Salt

To a suspension of compound obtained in example 22 (800 mg, 1.71 mmol, 1.0 eq.) in dichloromethane (40 mL) was added triethylamine (948 μL, 6.84 mmol, 4.0 eq.). The mixture was stirred at room temperature for 10 minutes before adding Boc-Gly-OSu (513 mg, 1.88 mmol, 1.1 eq.) and dichloromethane (12 mL). The reaction mixture was stirred at room temperature for 22 hours. A new addition of triethylamine (237 μL, 1.71 mmol, 1.0 eq.) and dichloromethane (8 mL) was performed after 16 hours stirring for further solubilization of the reaction mixture. The solvents were evaporated under reduced pressure, and the residue taken up in a mixture of EtOAc and water. The organic layer was successively washed with water, 1M NaOH, 1M HCl & brine, dried over Na₂SO₄, filtered through a silica gel pad, eluted with EtOAc and evaporated under reduced pressure to afford the Boc-protected derivative. The product was directly used in the next step without further purification. The solid was taken up in dichloromethane (20 mL), the solution cooled down to 0° C. before adding 4M HCl in CPME (8 mL, 32 mmol) under nitrogen. The reaction mixture was stirred at room temperature for 5 hours, still under an inert atmosphere. The heterogeneous mixture was degassed and evaporated under reduced pressure. The residue was taken up and triturated in Et₂O. The mixture was stirred at room temperature for 2 hours and left overnight without agitation. The supernatant was discarded and the residual ether removed under reduced pressure to afford the titled compound (790 mg, 88%) as a white solid. 1H NMR (DMSO-d₆, 500 MHZ): δ (ppm): 0.92 (d, J=6.7 Hz, 6H), 1.30 (m, 4H), 1.53 (m, 4H), 1.88-1.96 (m, 4H), 2.06 (bd, 2H), 2.14 (t, J=7.3 Hz, 2H), 2.21 (m, 1H), 2.57 (m, 1H), 3.49 (q, J=5.9 Hz, 2H), 3.59 (m, 1H), 3.72 (s, 3H), 4.24 (d, J=5.9 Hz, 2H), 5.28-5.39 (m, 2H), 6.80 (dd, J=8.1 Hz, J=1.5 Hz, 1H), 6.97 (d, J=8.1 Hz, 1H), 6.98 (d, J=1.5 Hz, 1H), 8.07 (bs, 3H), 8.34 (m, 1H), 8.37 (m, 1H)

### Example 32

### Synthesis of 2-methoxy-4-{[(E)-8-methylnon-6-enamido]methyl}phenyl trans-4-(2-aminopropanamido)cyclohexane-1-carboxylate, HCl Salt

To a suspension of compound obtained in example 22 (800 mg, 1.71 mmol, 1.0 eq.) in dichloromethane (40 mL) was added triethylamine (948 μL, 6.84 mmol, 4.0 eq.). The mixture was stirred at room temperature for 10 minutes before adding Boc-B-Ala-OSu (538 mg, 1.88 mmol, 1.1 eq.) and dichloromethane (12 mL). The reaction mixture was stirred at room temperature for 22 hours. A new addition of triethylamine (237 μL, 1.71 mmol, 1.0 eq.), dichloromethane (8 mL) and acetonitrile (6 mL) was performed after 16 hours stirring for further solubilisation of the reaction mixture. The solvents were evaporated under reduced pressure, and the residue taken up in a mixture of dichloromethane and water. The organic layer was successively washed with water, 1M NaOH, 1M HCl & brine, dried over Na₂SO₄ and evaporated. The residue was taken up in a 90/10 DCM/MeOH mixture, filtered through a silica gel pad and eluted with the same mixture. The solvents were evaporated to afford the Boc-protected derivative. The product was directly used in the next step without further purification. The solid was taken up in dichloromethane (20 mL), the solution cooled down to 0° C. before adding 4M HCl in CPME (8 mL, 32 mmol) under nitrogen. The reaction mixture was stirred at room temperature for 5 hours, still under an inert atmosphere. The heterogeneous mixture was degassed and evaporated under reduced pressure. The residue was taken up and triturated in Et₂O. The mixture was stirred at room temperature for 2 hours and left overnight without agitation. The supernatant was discarded and the residual ether removed under reduced pressure to afford the titled compound (690 mg, 75%) as a white solid. ¹H NMR (DMSO-d₆, 500 MHz): δ (ppm): 0.93 (d, J=6.7 Hz, 6H), 1.28 (m, 4H), 1.51 (m, 4H), 1.88 (bd, 2H), 1.93 (m, 2H), 2.06 (bd, 2H), 2.14 (t, J=7.3 Hz, 2H), 2.21 (m, 1H), 2.45 (t, J=6.8 Hz, 2H), 2.54 (m, 1H), 2.97 (m, 2H), 3.55 (m, 1H), 3.72 (s, 3H), 4.23 (d, J=5.9 Hz, 2H), 5.28-5.39 (m, 2H), 6.80 (dd, J=8.1 Hz, J=1.5 Hz, 1H), 6.96 (d, J=8.1 Hz, 1H), 6.98 (d, J=1.5 Hz, 1H), 7.86 (bs, 3H), 8.07 (m, 1H), 8.33 (m, 1H)

### Example 33

### Synthesis of (E)-2-methoxy-4-[(8-methylnon-6-enamido)methyl]phenyl (3-aminopropanoyl)-L-prolinate, HCl Salt

To a solution of {3-[(tert-butoxycarbonyl)amino]propanoyl}-L-proline (1.79 g, 6.25 mmol, 1.9 eq.) in dichloromethane (20 mL) cooled at 0° C. was added EDC·HCl (1.89 g, 19.65 mmol, 3.0 eq.) and dichloromethane (4 mL). N-methylimidazole (260 μL, 3.27 mmol, 1.0 eq.) was added and the mixture was stirred at room temperature for 5 minutes before adding pure (E)-capsaicin (1.0 g, 3.27 mmol, 1.0 eq.) and dichloromethane (2 mL). The reaction mixture was stirred at room temperature for 20 hours under a nitrogen atmosphere. Then the solvents are evaporated under reduced pressure (water bath at 30° C.) and the crude was taken up in a mixture of Et₂O/EtOAc and water. The organic layer was successively washed with water, 1M NaOH, 1M HCl & brine, dried over Na₂SO₄, filtered through a silica gel pad, eluted with Et₂O then EtOAc and evaporated under reduced pressure to afford the Boc-protected derivative. The product was directly used in the next step without further purification. The oil was taken up in dichloromethane (12 mL) and EtOAc (8 mL), the solution cooled down to 0° C. before adding 4M HCl in CPME (12 mL, 48 mmol) under nitrogen. The reaction mixture was stirred at room temperature for 5 hours, still under an inert atmosphere. The heterogeneous mixture was degassed and evaporated under reduced pressure. The residue was taken up and triturated in Et₂O. The mixture was stirred at room temperature for 2 hours and left overnight without agitation. The supernatant was discarded and the ether removed under reduced pressure to afford the titled compound (1.18 g, 71%) as a white solid. 1H NMR (DMSO-d₆, 500 MHz): δ (ppm) main rotamer: 0.93 (d, J=6.7 Hz, 6H), 1.30 (m, 2H), 1.51 (m, 2H), 1.94 (m, 2H), 2.02 (m, eq. 2H main rotamer), 2.13 (t, J=7.3 Hz, 2H), 2.20 (m, 1H), 2.32 (m, 1H), 2.69 (m, eq. 2H main rotamer), 3.00 (m, 2H), 3.51-3.62 (m, 2H), 3.73 (s, eq. 3H main rotamer), 4.24 (d, J=5.9 Hz, eq. 2H main rotamer), 4.58 (dd, J=8.8 Hz, J=3.7 Hz, eq. 1H main rotamer), 5.28-5.39 (m, 2H), 6.81 (dd, J=8.3 Hz, J=1.5 Hz, eq. 1H main rotamer), 6.96 (d, J=8.3 Hz, eq. 1H main rotamer), 6.99 (d, J=1.2 Hz, eq. 1H main rotamer), 7.80 (bs, 3H), 8.34 (m, 1H)

### Example 34

### Synthesis of (E)-2-methoxy-4-[(8-methylnon-6-enamido)methyl]phenyl piperidine-2-carboxylate, HCl Salt

To a solution of 1-tert-Butoxycarbonylpiperidine-2-carboxylic acid (3.0 g, 13.10 mmol, 2.0 eq.) in dichloromethane (60 mL) cooled at 0° C. was added EDC·HCl (3.77 g, 19.65 mmol, 3.0 eq.), dichloromethane (10 mL) then N-methylimidazole (520 μL, 6.55 mmol, 1.0 eq.). The mixture was stirred at 0° C. for 5 minutes before adding pure (E)-capsaicin (2.0 g, 6.55 mmol, 1.0 eq.) and dichloromethane (16 mL). The reaction mixture was stirred at room temperature for 20 hours under a nitrogen atmosphere. Then the solvents are evaporated under reduced pressure (water bath at 30° C.) and the crude was taken up in a mixture of Et₂O/EtOAc and water. The organic layer was successively washed with water, 1 M NaOH, 1 M HCl & brine, dried over Na₂SO₄, filtered through a silica gel pad, eluted with Et₂O and evaporated under reduced pressure to afford the Boc-protected derivative. The product was directly used in the next step without further purification. The solid was taken up in dichloromethane (12 mL) and EtOAc (12 mL), the solution cooled down to 0° C. before adding 4M HCl in CPME (26 mL, 104 mmol) under nitrogen. The reaction mixture was stirred at room temperature for 6 hours, still under an inert atmosphere. The heterogeneous mixture was degassed, and the precipitate was filtered, then washed with a small amount of dichloromethane and triturated with Et₂O. The residue was resuspended in diethyl ether, stirred overnight at room temperature then left for decantation. The supernatant was removed, the solid filtered and washed with diethyl ether to afford the titled compound (3.0 g, 99%) as an off-white solid. 1H NMR (DMSO-d₆, 500 MHZ): δ (ppm): 0.93 (d, J=6.7 Hz, 6H), 1.30 (m, 3H), 1.52 (m, 2H), 1.66 (m, 2H), 1.72-1.88 (m, 3H), 1.94 (m, 2H), 2.15 (t, J=7.3 Hz, 2H), 2.21 (m, 1H), 2.26 (m, 1H), 3.76 (s, 3H), 4.26 (d, J=5.9 Hz, 2H), 4.42 (m, 1H), 5.28-5.39 (m, 2H), 6.86 (dd, J=8.1 Hz, J=1.2 Hz, 1H), 7.6 (d, J=1.2 Hz, 1), 7.09 (d, J=8.1 Hz, 1H), 6.98 (d, J=1.2 Hz, 1H), 8.39 (m, 1H), 9.30 (m, 1H), 9.65 (m, 1H)

### Example 35

### Synthesis of 2-methoxy-4-{[(E)-8-methylnon-6-enamido]methyl}phenyl trans-4-(2-aminoacetamido)cyclohexane-1-carboxylate-Introduced sodium hyaluronate

Sodium hyaluronate with an average molecular weight of 880 kDa (250 mg, 2.5 eq. disaccharide unit) was dissolved in 25 mL of water at room temperature for 30 minutes before the addition of 25 mL of absolute ethanol. The mixture was further stirred at room temperature for 15 minutes before a first pH adjustment with the addition of 1 drop of NaOH 1M. A solution of compound obtained in example 31 (131 mg, 0.25 mmol, 1.0 eq.) in H₂O/EtOH (2 mL/4 mL) was added and the reaction was stirred for 5 minutes before adding a solution of DMTMM chloride (69 mg, 0.25 mmol, 1.0 eq.) in H₂O (4 mL). The pH was again adjusted to 6-7 with 4 drops of NaOH 1M and the reaction was stirred at room temperature for 26 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation for 30 minutes. The supernatant was discarded and the precipitate filtered. The solid was successively washed and triturated with EtOH 100%, EtOH 85% in water, EtOH 100%, acetone and Et₂O to afford the titled compound as a white meringue (180 mg after overnight drying). The content of capsaicin was in the 11-15% weight/weight range as determined by ultraviolet spectroscopy.

### Example 36

### Synthesis of 2-methoxy-4-{[(E)-8-methylnon-6-enamido]methyl}phenyl trans-4-(2-aminopropanamido)cyclohexane-1-carboxylate-Introduced sodium hyaluronate

Sodium hyaluronate with an average molecular weight of 880 kDa (250 mg, 2.5 eq. disaccharide unit) was dissolved in 25 mL of water at room temperature for 30 minutes before the addition of 25 mL of absolute ethanol. The mixture was further stirred at room temperature for 15 minutes before a first pH adjustment with the addition of 1 drop of NaOH 1M. A solution of example 32 (135 mg, 0.25 mmol, 1.0 eq.) in H₂O/EtOH (2 mL/2 mL) was added and the reaction was stirred for 5 minutes before adding a solution of DMTMM chloride (69 mg, 0.25 mmol, 1.0 eq.) in H₂O (4 mL). The pH was again adjusted to 6-7 with 4 drops of NaOH 1M and the reaction was stirred at room temperature for 26 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation for 30 minutes. The supernatant was discarded and the precipitate filtered. The solid was successively washed and triturated with EtOH 100%, EtOH 85% in water, EtOH 100%, acetone and Et₂O to afford the titled compound as a white meringue (200 mg after overnight drying). The content of capsaicin was in the 11-15% weight/weight range as by ultraviolet spectroscopy.

### Example 37

### Synthesis of (E)-2-methoxy-4-[(8-methylnon-6-enamido)methyl]phenyl glycyl-L-prolinate, HCl Salt

To a solution of (tert-butoxycarbonyl)glycyl-L-proline (870 mg, 3.20 mmol, 1.6 eq.) in dichloromethane (20 mL) cooled at 0° C. was added EDC·HCl (1.00 g, 5.20 mmol, 2.6 eq.) and dichloromethane (4 mL). N-methylimidazole (160 μL, 2.00 mmol, 1.0 eq.) was added and the mixture was stirred at room temperature for 5 minutes before adding pure (E)-capsaicin (611 mg, 2.00 mmol, 1.0 eq.) and dichloromethane (2 mL). The reaction mixture was stirred at room temperature for 15 hours under a nitrogen atmosphere. Then the solvents are evaporated under reduced pressure (water bath at 30° C.) and the crude was taken up in a mixture of Et₂O and water. The organic layer was successively washed with water, 1M NaOH, 1M HCl & brine, dried over Na₂SO₄, filtered through a silica gel pad, eluted with Et₂O then EtOAc. The Et₂O fraction was discarded and the EtOAc was evaporated under reduced pressure to afford the Boc-protected derivative. The product was directly used in the next step without further purification. The residue was taken up in dichloromethane (12 mL) and EtOAc (4 mL), the solution cooled down to 0° C. before adding 4M HCl in CPME (7 mL, 28 mmol) under nitrogen. The reaction mixture was stirred at room temperature for 2 hours, still under an inert atmosphere. The heterogeneous mixture was degassed and evaporated under reduced pressure. The residue was taken up and triturated in Et₂O. The mixture was stirred at room temperature for 2 hours and left overnight without agitation. The supernatant was discarded and the ether removed under reduced pressure to afford the titled compound (820 mg, 83%) as a white solid. 1H NMR (DMSO-d₆, 500 MHz): δ (ppm) main rotamer: 0.93 (d, J=6.7 Hz, 6H), 1.30 (m, 2H), 1.52 (m, 2H), 1.94 (m, 2H), 2.02 (m, eq. 2H main rotamer), 2.13 (t, J=7.3 Hz, 2H), 2.20 (m, 2H), 2.32 (m, 1H), 3.54 (m, 1H), 3.62 (m, 1H), 3.74 (s, eq. 3H main rotamer), 3.89 (m, eq. 2H main rotamer), 4.24 (d, J=5.9 Hz, eq. 2H main rotamer), 4.67 (dd, J=8.8 Hz, J=3.7 Hz, eq. 1H main rotamer), 5.28-5.39 (m, 2H), 6.82 (dd, J=8.3 Hz, J=1.5 Hz, eq. 1H main rotamer), 7.00 (d, J=1.2 Hz, eq. 1H main rotamer), 8.19 (bs, 3H), 8.34 (m, 1H)

### Example 38

### Synthesis of (E)-2-methoxy-4-[(8-methylnon-6-enamido)methyl]phenyl 1-glycylpiperidine-2-carboxylate, HCl Salt

To a suspension of compound obtained in example 34 (800 mg, 1.77 mmol, 1.0 eq.) in dichloromethane (20 mL) was added triethylamine (981 μL, 7.08 mmol, 4.0 eq.). The mixture was stirred at room temperature for 5 minutes before adding Boc-Gly-OSu (513 mg, 1.88 mmol, 1.1 eq.) and dichloromethane (10 mL). The reaction mixture was stirred at room temperature for 12 hours. The solvents were evaporated under reduced pressure, and the residue taken up in a mixture of Et₂O and water. The organic layer was successively washed with water, 1M NaOH, 1M HCl & brine, dried over Na₂SO₄ and filtered through a silica gel pad. Further elution was carried out with Et₂O and EtOAc, and the solvents were evaporated to afford the Boc-protected derivative. The product was directly used in the next step without further purification. The solid was taken up in dichloromethane (12 mL) and anhydrous ethyl acetate (4 mL), the solution cooled down to 0° C. before adding 4M HCl in CPME (7 mL, 28 mmol) under nitrogen. The reaction mixture was stirred at room temperature for 2 hours. The heterogeneous mixture was degassed and evaporated under reduced pressure. The residue was taken up and triturated in Et₂O. The mixture was stirred at room temperature for 1 hour and left overnight without agitation. The supernatant was discarded and the residual ether removed under reduced pressure to afford the titled compound (660 mg, 73%) as a white solid. ¹H NMR (DMSO-d₆, 500 MHZ): δ (ppm) main rotamer: 0.92 (d, J=6.7 Hz, 6H), 1.29 (m, 2H), 1.51 (m, 4H), 1.66-1.81 (m, 3H), 1.94 (m, 2H), 2.14 (t, J=7.3 Hz, 2H), 2.20 (m, 1H), 2.30 (m, 1H), 3.22 (m, eq. 1H main rotamer), 3.69 (m, 1H), 3.75 (s, eq. 3H main rotamer), 3.88 (m, eq. 1H main rotamer), 4.07 (m, eq. 1H main rotamer), 4.25 (d, J=5.9 Hz, 2H), 5.28-5.39 (m, 2H), 5.43 (bd, eq. 1H main rotamer), 6.82 (dd, J=8.3 Hz, eq. 1H main rotamer), 7.01 (d, J=8.3 Hz, eq. 1H main rotamer), 7.02 (bs, eq. 1H main rotamer), 8.16 (bs, 3H), 8.37 (m, 1H)

### Example 39

### Synthesis of (E)-2-methoxy-4-[(8-methylnon-6-enamido)methyl]phenyl 1-(3-aminopropanoyl) piperidine-2-carboxylate, HCl Salt

To a suspension of compound obtained in example 34 (800 mg, 1.77 mmol, 1.0 eq.) in dichloromethane (20 mL) was added triethylamine (981 μL, 7.08 mmol, 4.0 eq.). The mixture was stirred at room temperature for 5 minutes before adding Boc-B-Ala-OSu (707 mg, 2.47 mmol, 1.4 eq.) and dichloromethane (10 mL). The reaction mixture was stirred at room temperature for 12 hours. The solvents were evaporated under reduced pressure, and the residue taken up in a mixture of Et₂O and water. The organic layer was successively washed with water, 1M NaOH, 1M HCl & brine, dried over Na₂SO₄ and filtered through a silica gel pad. Further elution was carried out with Et₂O, and the solvent was evaporated to afford the Boc-protected derivative. The product was directly used in the next step without further purification. The solid was taken up in dichloromethane (12 mL) and anhydrous ethyl acetate (4 mL), the solution cooled down to 0° C. before adding 4M HCl in CPME (7 mL, 28 mmol) under nitrogen. The reaction mixture was stirred at room temperature for 2 hours. The heterogeneous mixture was degassed and evaporated under reduced pressure. The residue was taken up and triturated in Et₂O. The mixture was stirred at room temperature for 1 hour and left overnight without agitation. The supernatant was discarded and the residual ether removed under reduced pressure to afford the titled compound (550 mg, 59%) as a white solid. 1H NMR (DMSO-d₆, 500 MHZ): δ (ppm) main rotamer: 0.92 (d, J=6.7 Hz, 6H), 1.30 (m, 2H), 1.45 (m, 2H), 1.52 (m, 2H), 1.63-1.78 (m, 3H), 1.94 (m, 2H), 2.14 (t, J=7.3 Hz, 2H), 2.21 (m, 1H), 2.29 (m, 1H), 2.67-2.72 (m, eq. 1H main rotamer), 2.84 (m, eq. 1H main rotamer), 3.00 (m, 2H), 3.22 (m, eq. 1H main rotamer), 3.75 (s, eq. 3H main rotamer), 3.78 (m, eq. 1H main rotamer), 4.24 (d, J=5.9 Hz, 2H), 5.28-5.39 (m, 2H), 5.45 (bd, eq. 1H main rotamer), 6.82 (dd, J=8.3 Hz, eq. 1H main rotamer), 7.00 (d, J=8.3 Hz, eq. 1H main rotamer), 7.02 (bs, eq. 1H main rotamer), 7.84 (bs, 3H), 8.36 (m, 1H)

### Example 40

### Synthesis of (E)-2-methoxy-4-[(8-methylnon-6-enamido)methyl]phenyl piperidine-2-carboxylate-Introduced sodium hyaluronate

Sodium hyaluronate with an average molecular weight of 880 kDa (250 mg, 1.6 eq. disaccharide unit) was dissolved in 25 mL of water at room temperature for 20 minutes before the addition of 25 mL of absolute ethanol. The mixture was further stirred at room temperature for 10 minutes before a first pH adjustment with the addition of 2 drops of NaOH 1M. A solution of compound obtained in example 34 (172 mg, 0.38 mmol, 1.0 eq.) in H₂O/EtOH (2 mL/2 mL) was added and the reaction was stirred for 5 minutes before adding a solution of DMTMM chloride (105 mg, 0.38 mmol, 1.0 eq.) in H₂O (4 mL). The pH was again adjusted to 6-7 with 5 drops of NaOH 1M. The reaction was stirred at room temperature for 42 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation for 60 minutes. The supernatant was discarded and the precipitate filtered. The solid was successively washed and triturated with EtOH 100%, EtOH 85% in water, EtOH 100%, acetone and Et₂O to afford the titled compound as a white meringue (230 mg after overnight drying). The content of capsaicin was in the 6-10% weight/weight range as determined by ultraviolet spectroscopy.

### Example 41

### Synthesis of (E)-2-methoxy-4-[(8-methylnon-6-enamido)methyl]phenyl (3-aminopropanoyl)-L-prolinate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 10 minutes before a first pH adjustment with the addition of 1 drop of NaOH 1M. A solution of compound obtained in example 33 (128 mg, 0.25 mmol, 1.0 eq.) in H₂O/EtOH (2 mL/2 mL) was added and the reaction was stirred for 5 minutes before adding a solution of DMTMM chloride (69 mg, 0.25 mmol, 1.0 eq.) in H₂O (4 mL). The pH was again adjusted to 6-7 with 4 drops of NaOH 1M. The reaction was stirred at room temperature for 32 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation for 60 minutes. The supernatant was discarded and the precipitate filtered. The solid was successively washed and triturated with EtOH 100%, EtOH 85% in water, EtOH 100%, acetone and Et₂O to afford the titled compound as a white meringue (260 mg after overnight drying). The content of capsaicin was in the 6-10% weight/weight range as determined by ultraviolet spectroscopy.

### Example 42

### Synthesis of (E)-2-methoxy-4-[(8-methylnon-6-enamido)methyl]phenyl glycyl-L-prolinate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 15 minutes before a first pH adjustment with the addition of 1 drop of NaOH 1M. A solution of compound obtained in example 37 (124 mg, 0.25 mmol, 1.0 eq.) in H₂O/EtOH (2 mL/2 mL) was added and the reaction was stirred for 5 minutes before adding a solution of DMTMM chloride (69 mg, 0.25 mmol, 1.0 eq.) in H₂O (4 mL). The pH was again adjusted to 6-7 with 4 drops of NaOH 1M. The reaction was stirred at room temperature for 25 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation for 30 minutes. The supernatant was discarded and the precipitate filtered. The solid was successively washed and triturated with EtOH 100%, EtOH 85% in water, EtOH 100%, acetone and Et₂O to afford the titled compound as a white cotton-like meringue (260 mg after overnight drying). The content of capsaicin was in the 1-5% weight/weight range as determined by ultraviolet spectroscopy.

### Example 43

### Synthesis of (E)-2-methoxy-4-[(8-methylnon-6-enamido)methyl]phenyl 1-glycylpiperidine-2-carboxylate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 15 minutes before a first pH adjustment with the addition of 1 drop of NaOH 1M. A solution of compound obtained in example 38 (128 mg, 0.25 mmol, 1.0 eq.) in H₂O/EtOH (2 mL/2 mL) was added and the reaction was stirred for 5 minutes before adding a solution of DMTMM chloride (69 mg, 0.25 mmol, 1.0 eq.) in H₂O (4 mL). The pH was again adjusted to 6-7 with 4 drops of NaOH 1M. The reaction was stirred at room temperature for 25 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation for 30 minutes. The supernatant was discarded and the precipitate filtered. The solid was successively washed and triturated with EtOH 100%, EtOH 85% in water, EtOH 100%, acetone and Et₂O to afford the titled compound as a white meringue (230 mg after overnight drying). The content of capsaicin was in the 1-5% weight/weight range as determined by ultraviolet spectroscopy.

### Example 44

### Synthesis of (E)-2-methoxy-4-[(8-methylnon-6-enamido)methyl]phenyl 1-(3-aminopropanoyl) piperidine-2-carboxylate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 10 minutes before a first pH adjustment with the addition of 1 drop of NaOH 1M. A solution of compound obtained in example 39 (131 mg, 0.25 mmol, 1.0 eq.) in H₂O/EtOH (2 mL/2 mL) was added and the reaction was stirred for 5 minutes before adding a solution of DMTMM chloride (69 mg, 0.25 mmol, 1.0 eq.) in H₂O (4 mL). The pH was again adjusted to 6-7 with 4 drops of NaOH 1M. The reaction was stirred at room temperature for 25 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation for 30 minutes. The supernatant was discarded and the precipitate filtered. The solid was successively washed and triturated with EtOH 100%, EtOH 85% in water, EtOH 100%, acetone and Et₂O to afford the titled compound as a white cotton-like meringue (230 mg after overnight drying). The content of capsaicin was in the 6-10% weight/weight range as determined by ultraviolet spectroscopy.

### Example 45

### Synthesis of (E)-2-methoxy-4-[(8-methylnon-6-enamido)methyl]phenyl 2-[2-(2-aminoethoxy) ethoxy]acetate, TFA salt

To a solution of N-(tert-Butoxycarbonyl)glycine (191 mg, 1.09 mmol, 1.5 eq.) in N-methylpyrrolidinone (10 mL) cooled at 0° C. was added COMU (560 mg, 1.30 mmol, 1.8 eq.) then DIPEA (379 μL, 2.18 mmol, 3.0 eq.). The mixture was stirred at 0° C. for 5 minutes before adding a solution of compound obtained in example 2 (300 mg, 0.73 mmol, 1.0 eq.) in N-methylpyrrolidinone (10 mL). The resulting mixture was stirred at room temperature for 6 hours. The reaction was quenched with water and extracted with EtOAc. The organic layer was successively washed with saturated NH₄Cl, 0.5 M HCl, saturated Na₂CO₃ & brine, dried over Na₂SO₄, filtered through a silica gel pad and evaporated under reduced pressure to afford the Boc-protected derivative as an orange oil. The oil was taken up in dichloromethane (5 mL) and the solution cooled down to 0° C. before adding TFA (452 μL, 5.91 mmol). The reaction mixture was stirred at room temperature for 4 hours under a nitrogen atmosphere. The mixture was diluted with methanol, filtered and evaporated. The oil was taken up in methanol and evaporated three additional times to afford the titled compound (190 mg, 88%) as a clear oil after further drying under high vacuum. 1H NMR (DMSO-d₆, 500 MHz): δ (ppm): 0.93 (d, J=6.6 Hz, 6H), 1.29 (m, 2H), 1.52 (m, 2H), 1.94 (q, J=6.6 Hz, 2H), 2.14 (t, J=7.3 Hz, 2H), 2.21 (m, 1H), 2.77 (t, J=6.7 Hz, 2H), 3.45 (m, 2H), 3.54 (m, 2H), 3.74 (s, 3H), 4.25 (d, J=5.9 Hz, 2H), 5.27-5.40 (m, 2H), 6.81 (dd, J=8.1 Hz, J=1.6 Hz, 1H), 6.99 (d, J=1.6 Hz, 1H), 7.02 (d, J=8.1 Hz, 1H), 7.99 (bs, 3H), 8.32 (m, 1H), 8.53 (m, 1H)

### Example 46

### Synthesis of (E)-2-methoxy-4-[(8-methylnon-6-enamido)methyl]phenyl 2-[2-(2-aminoethoxy)ethoxy]acetate-Introduced sodium hyaluronate

Sodium hyaluronate with an average molecular weight of 880 kDa (146 mg, 2.5 eq. disaccharide unit) was dissolved in 15 mL of water at room temperature for 15 minutes before the addition of 15 mL of absolute ethanol. The mixture was further stirred at room temperature for 10 minutes before a first pH adjustment with the addition of 1 drop of NaOH 1M. A solution of compound obtained in example 45 (30 mg, 0.15 mmol, 1.0 eq.) in H₂O/EtOH (1 mL/1 mL) was added and the reaction was stirred for 10 minutes. A solution of DMTMM chloride (40 mg, 0.15 mmol, 1.0 eq.) in H₂O (2 mL) was added and the pH was again adjusted to 6-7 with 2 drops of NaOH 1M. The reaction was stirred at room temperature for 25 hours. NaCl (0.29 g) was then added. The slurry was stirred at room temperature for 30 minutes until solubilisation of the salt and poured into 200 mL of EtOH. The resulting precipitate was stirred in the EtOH/H₂O mixture at room temperature for 15 minutes, and left for decantation. The supernatant was discarded and the precipitate filtered. The solid was successively washed and triturated with EtOH 100%, EtOH 85% in water, EtOH 100%, acetone and Et₂O to afford the titled compound as a white meringue (145 mg after overnight drying). The content of capsaicin was in the 11-15% weight/weight range as determined by ultraviolet spectroscopy.

### Example 47

### Synthesis of (E)-2-methoxy-4-[(8-methylnon-6-enamido)methyl]phenyl glycinate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 10 minutes before a first pH adjustment with the addition of 1 drop of NaOH 1M. A solution of compound obtained in example 1 (100 mg, 0.25 mmol, 1.0 eq.) in H₂O/EtOH (1 mL/1 mL) was added and the reaction was stirred for 10 minutes. A solution of DMTMM chloride (70 mg, 0.25 mmol, 1.0 eq.) in H₂O (2 mL) was added and the pH was again adjusted to 6-7 with 7 drops of NaOH 1M. The reaction was stirred at room temperature for 25 hours. NaCl (0.5 g) was then added. The slurry was stirred at room temperature for 30 minutes until solubilisation of the salt and poured into 300 mL of EtOH. The resulting precipitate was stirred in the EtOH/H₂O mixture at room temperature for 15 minutes, and left for decantation. The supernatant was discarded and the precipitate filtered. The solid was successively washed and triturated with EtOH 100%, EtOH 85% in water, EtOH 100%, acetone and Et₂O to afford the titled compound as a white meringue (265 mg after overnight drying). The content of capsaicin was in the 6-10% weight/weight range as determined by ultraviolet spectroscopy.

### Example 48

### Synthesis of (E)-2-methoxy-4-[(8-methylnon-6-enamido)methyl]phenyl L-valinate, HCl Salt

To a solution of N-(tert-Butoxycarbonyl) valine (2.8 g, 13.10 mmol, 2.0 eq.) in dichloromethane (50 mL) cooled at 0° C. was added EDC·HCl (3.77 g, 19.65 mmol, 3.0 eq.) then DMAP (800 mg, 6.55 mmol, 1.0 eq.). The mixture was stirred at 0° C. for 10 minutes before adding pure (E)-capsaicin (2.0 g, 6.55 mmol, 1.0 eq.) and dichloromethane (10 mL). The reaction mixture was stirred at room temperature for 20 hours under a nitrogen atmosphere. Then the solvents are evaporated under reduced pressure and the crude was taken up in a mixture of EtOAc and water. The organic layer was successively washed with water, 0.5 M NaOH, 0.5 M HCl & brine, dried over Na₂SO₄, filtered through a silica gel pad, eluted with EtOAc and evaporated under reduced pressure to afford the Boc-protected derivative as an oil. The product was directly used in the next step without further purification. The oil was taken up in dichloromethane (15 mL) and EtOAc (15 mL), the solution cooled down to 0° C. before adding 1M HCl in Et₂O (65 mL, 65 mmol) under nitrogen. The reaction mixture was stirred at room temperature for 8 hours, still under an inert atmosphere. The heterogeneous mixture was degassed and partially evaporated under reduced pressure. The supernatant was discarded and the residue triturated with Et₂O. The mixture was stirred at room temperature for 2 hours and left overnight without agitation. The ether was removed under reduced pressure to afford the title compound (2.50 g, 86%) as a white solid. 1H NMR (DMSO-d₆, 500 MHz): 1H NMR (DMSO-d₆, 500 MHZ): δ (ppm): 0.92 (d, J=6.7 Hz, 6H), 1.11 (dd, J=12.7 Hz, J=6.8 Hz, 6H), 1.30 (m, 2H), 1.52 (m, 2H), 1.63 (m, 1H), 1.94 (m, 2H), 2.15 (t, J=7.3 Hz, 2H), 2.20 (m, 1H), 2.36 (m, 1H), 3.74 (s, 3H), 4.17 (m, 1H), 4.26 (d, J=5.4 Hz, 2H), 5.28-5.40 (m, 2H), 6.86 (d, J=8.3 Hz, 1H), 7.05 (bs, 1H), 7.09 (d, J=8.3 Hz, 1H), 8.40 (m, 1H), 8.74 (bs, 3H)

### Example 49

### Synthesis of (E)-2-methoxy-4-[(8-methylnon-6-enamido)methyl]phenyl L-valinate-Introduced sodium hyaluronate

Sodium hyaluronate with an average molecular weight of 880 kDa (250 mg, 1.25 eq. disaccharide unit) was dissolved in 25 mL of water at room temperature for 20 minutes before the addition of 25 mL of absolute ethanol. The mixture was further stirred at room temperature for 10 minutes before a first pH adjustment with the addition of 2 drops of NaOH 1M. A solution of compound obtained in example 48 (220 mg, 0.50 mmol, 1.0 eq.) in H₂O/EtOH (1 mL/1 mL) was added and the reaction was stirred for 10 minutes before a second pH adjustment with the addition of 2 drops of NaOH 1M. Then, a solution of DMTMM chloride (138 mg, 0.50 mmol, 1.0 eq.) in H₂O (2 mL) was added and the pH was again adjusted to 6-7 with 4 drops of NaOH 1M. The reaction was stirred at room temperature for 24 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation. The supernatant was discarded and the precipitate filtered. The solid was successively washed and triturated with EtOH 100%, EtOH 85% in water, EtOH 100%, acetone and Et₂O to afford the titled compound as a white meringue (260 mg after overnight drying). The content of capsaicin was in the 16-20% weight/weight range as determined by ultraviolet spectroscopy.

### Example 50

### Synthesis of (E)-2-methoxy-4-[(8-methylnon-6-enamido)methyl]phenyl (3-aminopropanoyl)-L-valinate, HCl Salt

To a solution of {3-[(tert-butoxycarbonyl)amino]propanoyl}-L-valine (1.34 g, 4.64 mmol, 2.0 eq.) in dichloromethane (30 mL) cooled at 0° C. was added EDC·HCl (1.30 g, 6.97 mmol, 3.0 eq.). DMAP (190 mg, 2.32 mmol, 1.0 eq.) was added and the mixture was stirred at room temperature for 5 minutes before adding pure (E)-capsaicin (708 mg, 2.32 mmol, 1.0 eq.) and dichloromethane (5 mL). The reaction mixture was stirred at room temperature for 6 hours under a nitrogen atmosphere. Then the solvents are evaporated under reduced pressure and the crude was taken up in a mixture of EtOAc and water. The organic layer was successively washed with water, 0.5M NaOH, 0.5M HCl & brine, dried over Na₂SO₄, filtered through a silica gel pad, eluted with EtOAc and evaporated under reduced pressure to afford the Boc-protected derivative. The product was directly used in the next step without further purification. The oil was taken up in dichloromethane (15 mL), the solution cooled down to 0° C. before adding TFA (1.5 mL, 20.0 mmol). The reaction mixture was stirred at room temperature for 4 hours under a nitrogen atmosphere. The mixture was diluted with methanol, filtered and evaporated. The oil was taken up in methanol and evaporated two additional times to afford the titled compound (730 mg, 53%) as an oil after further drying under high vacuum. ¹H NMR (DMSO-d₆, 500 MHZ): δ (ppm): 0.92 (d, J=6.7 Hz, 6H), 1.01 (t, J=7.1 Hz, 6H), 1.30 (m, 2H), 1.51 (m, 2H), 1.94 (m, 2H), 2.13 (t, J=7.3 Hz, 2H), 2.20 (m, 1H), 2.26 (m, 1H), 2.60 (m, 2H), 3.01 (m, 2H), 3.73 (s, 3H), 4.24 (d, J=5.9 Hz, 2H), 4.49 (dd, J=8.1 Hz, J=5.6 Hz, 1H), 5.28-5.39 (m, 2H), 6.82 (dd, J=8.1 Hz, 1H), 6.96 (d, J=8.1 Hz, 1H), 7.01 (bs, 1H), 7.71 (bs, 3H), 8.32 (m, 1H), 8.55 (m, 1H)

### Example 51

### Synthesis of (E)-2-methoxy-4-[(8-methylnon-6-enamido)methyl]phenyl (3-aminopropanoyl)-L-valinate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 10 minutes before a first pH adjustment with the addition of 1 drop of NaOH 1M. A solution of compound obtained in example 50 (147 mg, 0.25 mmol, 1.0 eq.) in H₂O/EtOH (1 mL/1 mL) was added and the reaction was stirred for 10 minutes. A solution of DMTMM chloride (69 mg, 0.25 mmol, 1.0 eq.) in H₂O (2 mL) was added and the pH was again adjusted to 6-7 with 5 drops of NaOH 1M. The reaction was stirred at room temperature for 24 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation. The supernatant was discarded and the precipitate filtered. The solid was successively washed and triturated with EtOH 100%, EtOH 85% in water, EtOH 100%, acetone and Et₂O to afford the titled compound as a white meringue (260 mg after overnight drying). The content of capsaicin was in the 6-10% weight/weight range as determined by ultraviolet spectroscopy.

### Example 52

### Synthesis of 2-methoxy-4-{[(E)-8-methylnon-6-enamido]methyl}phenyl (1S,2R)-2-aminocyclopentane-1-carboxylate, HCl Salt

To a solution of (1S,2R)-2-[(tert-Butoxycarbonyl)amino]cyclopentanecarboxylic acid (2.7 g, 11.80 mmol, 1.8 eq.) in dichloromethane (40 mL) cooled at 0° C. was added EDC·HCl (3.00 g, 15.72 mmol, 2.4 eq.). DMAP (640 mg, 5.24 mmol, 0.8 eq.) was added and the mixture was stirred at room temperature for 5 minutes before adding pure (E)-capsaicin (2.0 g, 6.55 mmol, 1.0 eq.) and dichloromethane (10 mL). The reaction mixture was stirred at room temperature for 20 hours under a nitrogen atmosphere. Then the solvents are evaporated under reduced pressure and the crude was taken up in a mixture of EtOAc and water. The organic layer was successively washed with water, 0.5M NaOH, 0.5M HCl & brine, dried over Na₂SO₄, and filtered through a silica gel pad. Ethyl acetate was evaporated under reduced pressure to afford the Boc-protected derivative as a white solid. The product was directly used in the next step without further purification. The residue was taken up in dichloromethane (10 mL) and EtOAc (10 mL), the solution cooled down to 0° C. before adding 4M HCl in CPME (20 mL, 80 mmol) under nitrogen. The reaction mixture was stirred at room temperature for 5 hours, still under an inert atmosphere. The heterogeneous mixture was degassed and evaporated under reduced pressure. The residue was taken up and triturated in Et₂O. The mixture was stirred at room temperature for 2 hours and left overnight without agitation. The supernatant was discarded and the ether removed under reduced pressure to afford the titled compound (1.64 g, 55%) as a white solid. ¹H NMR (DMSO-d₆, 500 MHZ): δ (ppm): 0.93 (d, J=6.7 Hz, 6H), 1.30 (m, 2H), 1.52 (m, 2H), 1.68 (m, 1H), 1.78-1.86 (m, 2H), 1.95 (m, 2H), 2.03 (m, 1H), 2.10 (m, 2H), 2.15 (m, 2H), 2.20 (m, 1H), 3.36 (m, 1H), 3.75 (s, 3H), 3.78 (m, 1H), 4.25 (d, J=5.9 Hz, 2H), 5.29-5.39 (m, 2H), 6.84 (dd, J=8.1 Hz, 1H), 7.01 (bs, 1H), 7.13 (d, J=8.1 Hz, 1H), 8.05 (bs, 3H), 8.35 (m, 1H)

### Example 53

### 2-methoxy-4-{[(E)-8-methylnon-6-enamido]methyl}phenyl (1S,2R)-2-aminocyclopentane-1-carboxylate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 10 minutes before a first pH adjustment with the addition of 1 drop of NaOH 1M. A solution of compound obtained in example 52 (113 mg, 0.25 mmol, 1.0 eq.) in H₂O/EtOH (1 mL/1 mL) was added and the reaction was stirred for 10 minutes. A solution of DMTMM chloride (69 mg, 0.25 mmol, 1.0 eq.) in H₂O (2 mL) was added and the pH was again adjusted to 6-7 with 5 drops of NaOH 1M. The reaction was stirred at room temperature for 24 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes, and left for decantation. The supernatant was discarded and the precipitate filtered. The solid was successively washed and triturated with EtOH 100%, EtOH 85% in water, EtOH 100%, acetone and Et₂O to afford the titled compound as a white meringue (250 mg after overnight drying). The content of capsaicin was in the 6-10% weight/weight range as determined by ultraviolet spectroscopy.

### Example 54

### Synthesis of (E)-2-methoxy-4-[(8-methylnon-6-enamido)methyl]phenyl 3-aminopropanoate-introduced sodium hyaluronate

Sodium hyaluronate with an average molecular weight of 880 kDa (500 mg, 1.25 eq. disaccharide unit) was stirred in H₂O/EtOH (50 mL/50 mL) for 90 minutes. A solution of compound obtained in example 2 (206 mg, 0.5 mmol, 1.0 eq.) in a H₂O/EtOH mixture (2 mL/2 mL) was added. The reaction was stirred for 10 minutes before adding a solution of DMTMM chloride (138 mg, 0.5 mmol, 1.0 eq.) in water (4 mL). The resulting mixture was stirred at room temperature for 29 hours. Then, NaCl (1.0 g) was added. The slurry was stirred at room temperature for 15 minutes until solubilisation of the salt and poured into 500 mL of EtOH. The resulting precipitate was stirred in the EtOH/H₂O mixture at room temperature for 15 minutes and left for decantation for 45 minutes. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 11-15% weight/weight range determined by ultraviolet spectroscopy.

### Example 55

### Synthesis of (E)-2-methoxy-4-[(8-methylnon-6-enamido)methyl]phenyl 3-(2-aminoethoxy)propanoate-Introduced sodium hyaluronate

Sodium hyaluronate with an average molecular weight of 880 kDa (500 mg, 2.5 eq. disaccharide unit) was dissolved in 50 mL of water at room temperature for 30 minutes before the addition of 50 mL of absolute ethanol. The mixture was further stirred at room temperature for 20 minutes and a solution of compound obtained in example 6 (229 mg, 1.0 mmol, 1.0 eq.) in a H₂O/EtOH mixture (2 mL/2 mL) was added. The reaction was stirred for 10 minutes before adding a solution of DMTMM chloride (138 mg, 0.5 mmol, 1.0 eq.) in water (4 mL). The resulting mixture was stirred at room temperature for 24 hours. NaCl (1.0 g) was then added. The slurry was stirred at room temperature for 10 minutes until solubilisation of the salt and poured into 600 mL of EtOH. The resulting precipitate was stirred in the EtOH/H₂O mixture at room temperature for 15 minutes and left for decantation for 30 minutes. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 6-10% weight/weight range as determined by ultraviolet spectroscopy.

### Example 56

### Synthesis of (E)-2-methoxy-4-[(8-methylnon-6-enamido)methyl]phenyl 3-(2-aminoethoxy)propanoate-Introduced sodium hyaluronate

Sodium hyaluronate with an average molecular weight of 880 kDa (250 mg, 1.6 eq. disaccharide unit) was dissolved in 25 mL of water at room temperature for 50 minutes before the addition of 25 mL of absolute ethanol. The mixture was further stirred at room temperature for 40 minutes. A solution of compound obtained in example 6 (172 mg, 0.38 mmol, 1.0 eq.) in H₂O/EtOH (2 mL/2 mL) was added and the reaction was stirred for 5 minutes. Then, a solution of DMTMM chloride (104 mg, 0.38 mmol, 1.0 eq.) in H₂O (4 mL) was added. The reaction was stirred at room temperature for 29 hours. NaCl (0.5 g) was then added. The slurry was stirred at room temperature for 30 minutes until solubilisation of the salt and poured into 300 mL of EtOH. The resulting precipitate was stirred in the EtOH/H₂O mixture at room temperature for 15 minutes and left for decantation for 40 minutes. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 6-10% weight/weight range as determined by ultraviolet spectroscopy.

### Example 57

### Synthesis of (E)-2-methoxy-4-[(8-methylnon-6-enamido)methyl]phenyl 3-(2-aminoethoxy)propanoate-Introduced sodium hyaluronate

Sodium hyaluronate with an average molecular weight of 880 kDa (250 mg, 1.6 eq. disaccharide unit) was dissolved in 25 mL of water at room temperature for 30 minutes before the addition of 25 mL of absolute ethanol. The mixture was further stirred at room temperature for 15 minutes. A solution of compound obtained in example 6 (172 mg, 0.38 mmol, 1.0 eq.) in H₂O/EtOH (2 mL/2 mL) was added and the reaction was stirred for 5 minutes. Then, a solution of DMTMM tetrafluoroborate (128 mg, 0.38 mmol, 1.0 eq.) in acetonitrile (4 mL) was added. The reaction was stirred at room temperature for 29 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation for 45 minutes. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 16-20% weight/weight range as determined by ultraviolet spectroscopy.

### Example 58

### Synthesis of (E)-2-methoxy-4-[(8-methylnon-6-enamido)methyl]phenyl [2-(2-aminoethoxy)ethyl]carbamate-Introduced sodium hyaluronate

Sodium hyaluronate with an average molecular weight of 880 kDa (250 mg, 1.6 eq. disaccharide unit) was dissolved in 25 mL of water at room temperature for 20 minutes before the addition of 25 mL of absolute ethanol. The mixture was further stirred at room temperature for 10 minutes. A solution of compound obtained in example 18 (179 mg, 0.38 mmol, 1.0 eq.) in H₂O/EtOH (2 mL/2 mL) was added and the reaction was stirred for 5 minutes. Then, a solution of DMTMM chloride (104 mg, 0.38 mmol, 1.0 eq.) in H₂O (4 mL) was added. The reaction was stirred at room temperature for 29 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 10 minutes and left for decantation for 15 minutes. The supernatant was discarded, and the precipitate filtered. The solid was successively washed and triturated with EtOH 100%, EtOH 85% in water, EtOH 100%, acetone and Et₂O to afford the titled compound as a white meringue/solid. The content of capsaicin was in the 11-15% weight/weight range as determined by ultraviolet spectroscopy.

### Example 59

### Synthesis of (E)-2-methoxy-4-[(8-methylnon-6-enamido)methyl]phenyl (5-aminopentyl)carbamate-Introduced sodium hyaluronate

Sodium hyaluronate with an average molecular weight of 880 kDa (250 mg, 1.6 eq. disaccharide unit) was dissolved in 25 mL of water at room temperature for 20 minutes before the addition of 25 mL of absolute ethanol. The mixture was further stirred at room temperature for 10 minutes. A solution of compound obtained in example 17 (179 mg, 0.38 mmol, 1.0 eq.) in H₂O/EtOH (2 mL/2 mL) was added and the reaction was stirred for 5 minutes. Then, a solution of DMTMM chloride (104 mg, 0.38 mmol, 1.0 eq.) in H₂O (4 mL) was added. The reaction was stirred at room temperature for 29 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 10 minutes and left for decantation for 15 minutes. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 11-15% weight/weight range determined by ultraviolet spectroscopy.

### Example 60

### Synthesis of (E)-2-methoxy-4-[(8-methylnon-6-enamido)methyl]phenyl 3-aminopyrrolidine-1-carboxylate-Introduced sodium hyaluronate

Sodium hyaluronate with an average molecular weight of 880 kDa (250 mg, 1.6 eq. disaccharide unit) was dissolved in 25 mL of water at room temperature for 20 minutes before the addition of 25 mL of absolute ethanol. The mixture was further stirred at room temperature for 5 minutes. A solution of compound obtained in example 23 (197 mg, 0.38 mmol, 1.0 eq.) in H₂O/EtOH (2 mL/2 mL) was added and the reaction was stirred for 5 minutes. Then, a solution of DMTMM chloride (104 mg, 0.38 mmol, 1.0 eq.) in H₂O (4 mL) was added. The reaction was stirred at room temperature for 50 hours. NaCl (0.5 g) was then added. The slurry was stirred at room temperature for 20 minutes until solubilisation of the salt and poured into 300 mL of EtOH. The resulting precipitate was stirred in the EtOH/H₂O mixture at room temperature for 20 minutes and left for decantation for 60 minutes. The supernatant was discarded, and the precipitate filtered. The solid was successively washed and triturated with EtOH 100%, EtOH 85% in water, EtOH 100%, acetone and Et₂O to afford the titled compound as a white solid. The content of capsaicin was in the 16-20% weight/weight range as determined by ultraviolet spectroscopy.

### Example 61

### Synthesis of 2-methoxy-4-{[(E)-8-methylnon-6-enamido]methyl]phenyl trans-4-aminocyclohexane-1-carboxylate-Introduced sodium hyaluronate

Sodium hyaluronate with an average molecular weight of 880 kDa (250 mg, 1.4 eq. disaccharide unit) was dissolved in 27 mL of water at room temperature for 30 minutes before the addition of 25 mL of absolute ethanol. The mixture was further stirred at room temperature for 15 minutes. A solution of compound obtained in example 22 (207 mg, 0.44 mmol, 1.0 eq.) in H₂O/EtOH (2 mL/2 mL) was added and the reaction was stirred for 5 minutes. Then, a solution of DMTMM tetrafluoroborate (145 mg, 0.44 mmol, 1.0 eq.) in H₂O/acetonitrile (1 mL/3 mL) was added. The reaction was stirred at room temperature for 46 hours. NaCl (0.5 g) was then added. The slurry was stirred at room temperature for 25 minutes until solubilisation of the salt and poured into 300 mL of EtOH. The resulting precipitate was stirred in the EtOH/H₂O mixture at room temperature for 25 minutes and left for decantation for 30 minutes. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 1-5% weight/weight range as determined by ultraviolet spectroscopy.

### Example 62

### Synthesis of 2-methoxy-4-{[(E)-8-methylnon-6-enamido]methyl}phenyl L-isoleucinate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 20 minutes. A solution of compound obtained in example 28 (114 mg, 0.25 mmol, 1.0 eq.) in H₂O/EtOH (2 mL/2 mL) was added and the reaction was stirred for 5 minutes before adding a solution of DMTMM chloride (69 mg, 0.25 mmol, 1.0 eq.) in H₂O (4 mL). The reaction was stirred at room temperature for 44 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation for 15 minutes. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 11-15% weight/weight range as determined by ultraviolet spectroscopy.

### Example 63

### Synthesis of (E)-2-methoxy-4-[(8-methylnon-6-enamido)methyl]phenyl (2-aminoethyl)(isopropyl)carbamate-Introduced sodium hyaluronate

Sodium hyaluronate with an average molecular weight of 880 kDa (250 mg, 1.6 eq. disaccharide unit) was dissolved in 25 mL of water at room temperature for 20 minutes before the addition of 25 mL of absolute ethanol. The mixture was further stirred at room temperature for 15 minutes. A solution of compound obtained in example 27 (179 mg, 0.38 mmol, 1.0 eq.) in H₂O/EtOH (2 mL/2 mL) was added and the reaction was stirred for 5 minutes. Then, a solution of DMTMM chloride (104 mg, 0.38 mmol, 1.0 eq.) in H₂O (4 mL) was added. The reaction was stirred at room temperature for 48 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation for 15 minutes. The supernatant was discarded, and the precipitate filtered. The solid was successively washed and triturated with EtOH 100%, EtOH 85% in water, EtOH 100%, acetone and Et₂O to afford the titled compound as a white solid. The content of capsaicin was in the 21-25% weight/weight range as determined by ultraviolet spectroscopy.

### Example 64

### Synthesis of 2-methoxy-4-{[(E)-8-methylnon-6-enamido]methyl}phenyl trans-4-(2-aminoacetamido)cyclohexane-1-carboxylate-Introduced sodium hyaluronate

Sodium hyaluronate with an average molecular weight of 880 kDa (250 mg, 2.5 eq. disaccharide unit) was dissolved in 25 mL of water at room temperature for 30 minutes before the addition of 25 mL of absolute ethanol. The mixture was further stirred at room temperature for 15 minutes. A solution of compound obtained in example 31 (131 mg, 0.25 mmol, 1.0 eq.) in H₂O/EtOH (2 mL/4 mL) was added and the reaction was stirred for 5 minutes before adding a solution of DMTMM chloride (69 mg, 0.25 mmol, 1.0 eq.) in H₂O (4 mL). The reaction was stirred at room temperature for 26 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation for 30 minutes. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 11-15% weight/weight range as determined by ultraviolet spectroscopy.

### Example 65

### Synthesis of 2-methoxy-4-{[(E)-8-methylnon-6-enamido]methyl}phenyl trans-4-(2-aminopropanamido)cyclohexane-1-carboxylate-Introduced sodium hyaluronate

Sodium hyaluronate with an average molecular weight of 880 kDa (250 mg, 2.5 eq. disaccharide unit) was dissolved in 25 mL of water at room temperature for 30 minutes before the addition of 25 mL of absolute ethanol. The mixture was further stirred at room temperature for 15 minutes. A solution of example 32 (135 mg, 0.25 mmol, 1.0 eq.) in H₂O/EtOH (2 mL/2 mL) was added and the reaction was stirred for 5 minutes before adding a solution of DMTMM chloride (69 mg, 0.25 mmol, 1.0 eq.) in H₂O (4 mL). The reaction was stirred at room temperature for 26 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation for 30 minutes. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 11-15% weight/weight range as by ultraviolet spectroscopy.

### Example 66

### Synthesis of (E)-2-methoxy-4-[(8-methylnon-6-enamido)methyl]phenyl piperidine-2-carboxylate-Introduced sodium hyaluronate

Sodium hyaluronate with an average molecular weight of 880 kDa (250 mg, 1.6 eq. disaccharide unit) was dissolved in 25 mL of water at room temperature for 20 minutes before the addition of 25 mL of absolute ethanol. The mixture was further stirred at room temperature for 10 minutes. A solution of compound obtained in example 34 (172 mg, 0.38 mmol, 1.0 eq.) in H₂O/EtOH (2 mL/2 mL) was added and the reaction was stirred for 5 minutes before adding a solution of DMTMM chloride (105 mg, 0.38 mmol, 1.0 eq.) in H₂O (4 mL). The reaction was stirred at room temperature for 42 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation for 60 minutes. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 6-10% weight/weight range as determined by ultraviolet spectroscopy.

### Example 67

### Synthesis of (E)-2-methoxy-4-[(8-methylnon-6-enamido)methyl]phenyl (3-aminopropanoyl)-L-prolinate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 10 minutes. A solution of compound obtained in example 33 (128 mg, 0.25 mmol, 1.0 eq.) in H₂O/EtOH (2 mL/2 mL) was added and the reaction was stirred for 5 minutes before adding a solution of DMTMM chloride (69 mg, 0.25 mmol, 1.0 eq.) in H₂O (4 mL). The reaction was stirred at room temperature for 32 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation for 60 minutes. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 6-10% weight/weight range as determined by ultraviolet spectroscopy.

### Example 68

### Synthesis of (E)-2-methoxy-4-[(8-methylnon-6-enamido)methyl]phenyl glycyl-L-prolinate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 15 minutes. A solution of compound obtained in example 37 (124 mg, 0.25 mmol, 1.0 eq.) in H₂O/EtOH (2 mL/2 mL) was added and the reaction was stirred for 5 minutes before adding a solution of DMTMM chloride (69 mg, 0.25 mmol, 1.0 eq.) in H₂O (4 mL). The reaction was stirred at room temperature for 25 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation for 30 minutes. The supernatant was discarded, and the precipitate filtered. The solid was successively washed and triturated with EtOH 100%, EtOH 85% in water, EtOH 100%, acetone and Et₂O to afford the titled compound as a white cotton-like meringue. The content of capsaicin was in the 1-5% weight/weight range as determined by ultraviolet spectroscopy.

### Example 69

### Synthesis of (E)-2-methoxy-4-[(8-methylnon-6-enamido)methyl]phenyl 1-glycylpiperidine-2-carboxylate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 15 minutes. A solution of compound obtained in example 38 (128 mg, 0.25 mmol, 1.0 eq.) in H₂O/EtOH (2 mL/2 mL) was added and the reaction was stirred for 5 minutes before adding a solution of DMTMM chloride (69 mg, 0.25 mmol, 1.0 eq.) in H₂O (4 mL). The reaction was stirred at room temperature for 25 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation for 30 minutes. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 1-5% weight/weight range as determined by ultraviolet spectroscopy.

### Example 70

### Synthesis of (E)-2-methoxy-4-[(8-methylnon-6-enamido)methyl]phenyl 1-(3-aminopropanoyl) piperidine-2-carboxylate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 10 minutes. A solution of compound obtained in example 39 (131 mg, 0.25 mmol, 1.0 eq.) in H₂O/EtOH (2 ml/2 mL) was added and the reaction was stirred for 5 minutes before adding a solution of DMTMM chloride (69 mg, 0.25 mmol, 1.0 eq.) in H₂O (4 mL). The reaction was stirred at room temperature for 25 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation for 30 minutes. The supernatant was discarded, and the precipitate filtered. The solid was successively washed and triturated with EtOH 100%, EtOH 85% in water, EtOH 100%, acetone and Et₂O to afford the titled compound as a white cotton-like meringue. The content of capsaicin was in the 6-10% weight/weight range as determined by ultraviolet spectroscopy.

### Example 71

### Synthesis of (E)-2-methoxy-4-[(8-methylnon-6-enamido)methyl]phenyl 2-[2-(2-aminoethoxy)ethoxy]acetate-Introduced sodium hyaluronate

Sodium hyaluronate with an average molecular weight of 880 kDa (146 mg, 2.5 eq. disaccharide unit) was dissolved in 15 mL of water at room temperature for 15 minutes before the addition of 15 mL of absolute ethanol. The mixture was further stirred at room temperature for 10 minutes. A solution of compound obtained in example 45 (30 mg, 0.15 mmol, 1.0 eq.) in H₂O/EtOH (1 mL/1 mL) was added and the reaction was stirred for 10 minutes. A solution of DMTMM chloride (40 mg, 0.15 mmol, 1.0 eq.) in H₂O (2 mL) was added. The reaction was stirred at room temperature for 25 hours. NaCl (0.29 g) was then added. The slurry was stirred at room temperature for 30 minutes until solubilisation of the salt and poured into 200 mL of EtOH. The resulting precipitate was stirred in the EtOH/H₂O mixture at room temperature for 15 minutes and left for decantation. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 11-15% weight/weight range as determined by ultraviolet spectroscopy.

### Example 72

### Synthesis of (E)-2-methoxy-4-[(8-methylnon-6-enamido)methyl]phenyl glycinate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 10 minutes. A solution of compound obtained in example 1 (100 mg, 0.25 mmol, 1.0 eq.) in H₂O/EtOH (1 mL/1 mL) was added and the reaction was stirred for 10 minutes. A solution of DMTMM chloride (70 mg, 0.25 mmol, 1.0 eq.) in H₂O (2 mL) was added. The reaction was stirred at room temperature for 25 hours. NaCl (0.5 g) was then added. The slurry was stirred at room temperature for 30 minutes until solubilisation of the salt and poured into 300 mL of EtOH. The resulting precipitate was stirred in the EtOH/H₂O mixture at room temperature for 15 minutes and left for decantation. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 6-10% weight/weight range as determined by ultraviolet spectroscopy.

### Example 73

### Synthesis of (E)-2-methoxy-4-[(8-methylnon-6-enamido)methyl]phenyl L-valinate-Introduced sodium hyaluronate

Sodium hyaluronate with an average molecular weight of 880 kDa (250 mg, 1.25 eq. disaccharide unit) was dissolved in 25 mL of water at room temperature for 20 minutes before the addition of 25 mL of absolute ethanol. The mixture was further stirred at room temperature for 10 minutes. A solution of compound obtained in example 48 (220 mg, 0.50 mmol, 1.0 eq.) in H₂O/EtOH (1 mL/1 mL) was added and the reaction was stirred for 10 minutes. Then, a solution of DMTMM chloride (138 mg, 0.50 mmol, 1.0 eq.) in H₂O (2 mL) was added. The reaction was stirred at room temperature for 24 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 16-20% weight/weight range as determined by ultraviolet spectroscopy.

### Example 74

### Synthesis of (E)-2-methoxy-4-[(8-methylnon-6-enamido)methyl]phenyl (3-aminopropanoyl)-L-valinate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 10 minutes. A solution of compound obtained in example 50 (147 mg, 0.25 mmol, 1.0 eq.) in H₂O/EtOH (1 mL/1 mL) was added and the reaction was stirred for 10 minutes. A solution of DMTMM chloride (69 mg, 0.25 mmol, 1.0 eq.) in H₂O (2 mL) was added. The reaction was stirred at room temperature for 24 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 6-10% weight/weight range as determined by ultraviolet spectroscopy.

### Example 75

### 2-methoxy-4-{[(E)-8-methylnon-6-enamido]methyl}phenyl (1S,2R)-2-aminocyclopentane-1-carboxylate-Introduced sodium hyaluronate

Sodium hyaluronate with an average molecular weight of 880 kDa (250 mg, 2.5 eq. disaccharide unit) was dissolved in 25 mL of water at room temperature for 20 minutes before the addition of 25 mL of absolute ethanol. The mixture was further stirred at room temperature for 10 minutes. A solution of compound obtained in example 52 (113 mg, 0.25 mmol, 1.0 eq.) A solution of DMTMM chloride (69 mg, 0.25 mmol, 1.0 eq.) in H₂O (2 mL) was added. The reaction was stirred at room temperature for 24 hours. NaCl (0.5 g) was then added. The slurry was stirred at room temperature for 15 minutes until solubilisation of the salt and poured into 300 mL of EtOH. The resulting precipitate was stirred in the EtOH/H₂O mixture at room temperature for 15 minutes and left for decantation. The supernatant was discarded, and the precipitate filtered. The solid was successively washed and triturated with EtOH 100%, EtOH 85% in water, EtOH 100%, acetone and Et₂O to afford the titled compound as a white meringue. The content of capsaicin was in the 6-10% weight/weight range as determined by ultraviolet spectroscopy.

### Example 76

### Synthesis of (E)-2-methoxy-4-[(8-methylnon-6-enamido)methyl]phenyl (3-aminopropanoyl)-D-prolinate, HCl Salt

Title compound was prepared according to example 33 using {3-[(tert-butoxycarbonyl)amino]propanoyl}-D-proline.

### Example 77

### Synthesis of (E)-2-methoxy-4-[(8-methylnon-6-enamido)methyl]phenyl (3-aminopropanoyl)-D-prolinate-Introduced sodium hyaluronate

Title compound was prepared according to example 41 using compound obtained in example 33.

### Example 78

### Synthesis of (E)-2-methoxy-4-[(8-methylnon-6-enamido)methyl]phenyl (3-aminopropanoyl)-DL-prolinate, HCl Salt

Title compound was prepared according to example 33 using {3-[(tert-butoxycarbonyl)amino]propanoyl}-DL-proline.

### Example 79

### Synthesis of (E)-2-methoxy-4-[(8-methylnon-6-enamido)methyl]phenyl (3-aminopropanoyl)-DL-prolinate-Introduced sodium hyaluronate

Title compound was prepared according to example 41 using compound obtained in example 78.

### Physicochemical Characterisation of the Polymer-Ion Channel Modulator Conjugate

This details some of the characterisation tests for an exemplary final product described in example 11 which is hyaluronic acid (750 kDa molecular weight) with a content of capsaicin in the 11-15% weight/weight range. Similar characterisation was done for the other examples listed above.

### a) Zeta-Potential Analysis

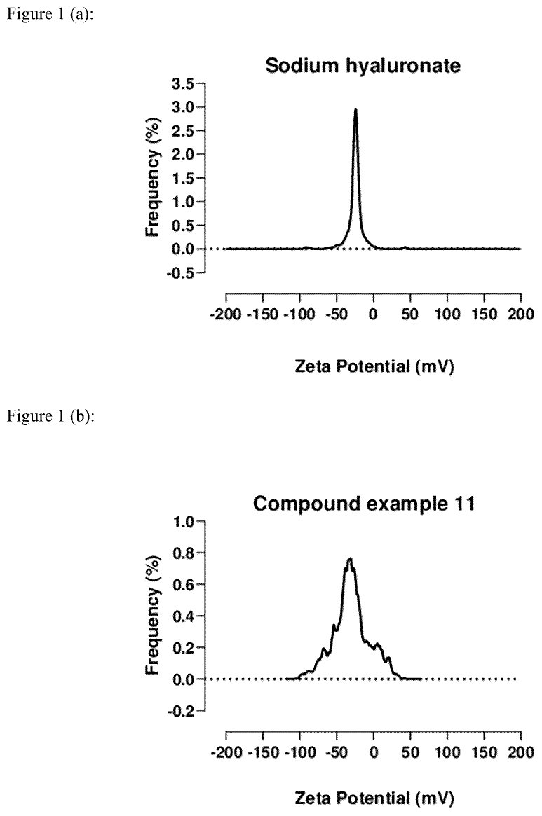

Zeta-potential is measure of the effective electric charge on the nanoparticle's surface, quantifying the charges. The mean zeta potential of sodium hyaluronate (1 mg/mL) was recorded at −23.2 mV and is shown in FIG. 1A. The mean zeta potential of the compound obtained in example 11 (namely (E)-2-methoxy-4-[(8-methylnon-6-enamido)methyl]phenyl 3-aminopropanoate-introduced sodium hyaluronate) (1 mg/mL) was recorded at −30.4 mV and shown in FIG. 1B. These results prove that the solution of compound example 11 is highly anionic and when compared with sodium hyaluronate, the composition has a higher negative value. A zeta potential of range +/−60 mV indicates stability in the case of colloid systems. In the case of the solution of compound example 11, while conjugation with capsaicin derivative may theoretically be expected to reduce the negative charge, surprisingly the opposite effect was seen to occur. This increased negative value compared to sodium hyaluronate may be due to a compaction of the molecule, leading to increased charge density on the surface. While conjugation with a capsaicin derivative might theoretically be expected to reduce the negative charge, the observed increase in zeta potential suggests that structural rearrangement or compaction could be concentrating the negative charge. The broadening of the zeta potential peak for the compound example 11 likely reflects increased heterogeneity in the surface charge distribution due to the conjugation process, including variations in conjugation efficiency, surface charge shielding, and particle size changes.

### b) Fourier Transform Infrared Spectroscopy (FTIR)

FTIR spectra of hyaluronic acid (HA), capsaicin, and the conjugate obtained in example 11 (in powder form) were recorded using a SHIMADZU-FTIR-8300 instrument over a spectral range of 4000-400 cm 1. All spectra were collected with 20-scan data accumulation at a resolution of 4 cm 1. This analysis enabled the identification of functional groups in the tested materials. The spectra for the conjugate in Example 11 displayed a distinct fingerprint, showing shared signals from both pristine HA and capsaicin, while demonstrating novelty through the presence and absence of specific characteristic peaks, in comparison to the simple mixture of HA and capsaicin.

### c) ¹H Nuclear Magnetic Resonance (NMR)

NMR spectroscopy was performed using an Agilent DD2 NMR 600 MHz instrument. This method was used to confirm the synthesis by identifying distinct signals, such as those for the aromatic protons of capsaicin. Additionally, NMR spectroscopy was employed quantitatively to confirm the conjugation percentage by comparing specific signals from both hyaluronic acid (HA) and capsaicin. Finally, NMR was also used to assess the purity of the manufactured conjugate.

### d) UV-Vis Spectroscopy

UV-Vis measurements were conducted using the NanoDrop 2000 Spectrophotometer from Thermo Fisher Scientific, specifically designed for small sample volumes. The maximum absorbance for capsaicinoids may vary depending on the capsaicinoid present and the solvent, and is described to be approximately 275-300 nm. UV-Vis was used to determine the content of capsaicinoid in our conjugate.

### Formulation of the Polymer-Ion Channel Modulator Conjugate

The conjugates prepared in the previous examples were assessed for solubility in various buffer systems, including phosphate buffered saline (PBS), 2-(N-morpholino) ethanesulfonic acid (MES) buffer, histidine buffer, citrate buffer, and phosphate buffer. Each buffer was carefully selected based on its compatibility with both the hyaluronic acid (HA) backbone and the TRPV1 agonist, aiming to optimize the solubility, stability, and delivery of the final formulation. Solubility tests revealed varying degrees of solubility across the buffers, influenced by both pH and ionic strength. Notably, PBS provided a relatively stable medium for most conjugates, but at concentrations of 10 mg/mL, solubility issues were encountered. To maintain the osmolarity of the formulation while overcoming solubility limitations, the sodium chloride component of PBS was replaced with PEG300, PEG400, propylene glycol, Tween 20, and Tween 80. Among these alternatives, PEG400 yielded the best results in terms of maintaining solubility, stability, and osmolarity within the desired range. MES and histidine buffers, with buffering capacities in the pH range of 5.5-6.5, were also explored as more acidic environments appeared to be preferable for the conjugates' stability. However, some caution was required, as TRPV1 channels are known to be sensitive to low pH; activation of TRPV1 has been reported in environments with pH values lower than 5.0, making it critical to maintain an optimal pH range. Citrate buffer was also considered, but it has been documented to activate the TRPV1 channel, leading to pain and discomfort when used in formulations intended for local injection, particularly at acidic pH levels. Similarly, histidine at certain concentrations has been shown to activate TRPV1, requiring careful concentration management. Phosphate buffer, with its broad pH range and buffering capacity, was tested in the same pH range as MES and histidine buffers. Although phosphate buffer demonstrated good solubility and stability for the conjugates, it also posed a potential risk for TRPV1 activation at higher concentrations, particularly when used in high molarity formulations. In addition to solubility, the viscosity of the conjugates was a key consideration, as it directly impacts the injectability and residence time at the target site. The conjugates formulated in different buffers exhibited increased viscosity compared to pristine HA, with values up to five times higher depending on the specific formulation. This increase in viscosity is attributed to the structural modifications introduced by conjugating the TRPV1 agonist to HA, as well as interactions between the conjugate and the buffer system.

For in vivo studies, the conjugates were primarily prepared in phosphate buffer at concentrations of 10 mg/mL, with a buffer concentration of 10-50 mM and a pH range of 5.0 to 7.5 (example 11). The osmolarity was carefully adjusted to 280-320 mOsm/kg using PEG400 or PEG300 to ensure physiological compatibility. The preferred pH range for these formulations was 6-7, offering an optimal balance between minimizing injection site pain (ISP), maintaining drug stability, and ensuring a controlled release of the TRPV1 agonist. By avoiding extreme pH values, the formulations were able to prevent undesirable TRPV1 activation, thus enhancing patient comfort while achieving the desired therapeutic effects.

### Sterilisation of the Polymer—Ion Channel Modulator Conjugate

The conjugates prepared in the previous examples were subjected to sterilization studies using dry heat sterilization, steam heat sterilization, and filtration sterilization. Each method was carefully evaluated to assess sterility as well as the impact on the structural integrity of the hyaluronic acid (HA) backbone and the TRPV1 agonist conjugate. Dry heat sterilization (160° C. for 120 minutes) was applied to the conjugates in powder form. NMR analysis revealed that the covalent bond between the TRPV1 agonist derivative and the HA backbone remained stable after treatment. However, the hyaluronic acid polymer base exhibited some degradation, evidenced by increased resolution in the NMR peaks, indicating a reduction in the molecular weight of HA. This reduction correlates with a smaller polymer size, affecting the overall structural integrity of the conjugate. Steam heat sterilization (121° C. for 15 minutes) was conducted on both powdered and solution forms of the conjugates (10 mg/mL in various buffers). In powder form, NMR and UV-Vis analysis confirmed that the capsaicin structure was preserved, and the covalent bond between the TRPV1 agonist derivative and the HA backbone remained stable. However, in solution form, some precipitation occurred, attributed to capsaicin release and subsequent precipitation. Importantly, the conjugate sterilized in powder form demonstrated the same in vitro release kinetics as the non-sterilized conjugate (example 11). Additional assessments confirmed a bioburden of <1 CFU/g and endotoxin levels <0.5 EU/mg, validating the steam heat sterilization method. Filtration sterilization was explored using polyethersulfone (PES), cellulose acetate (CA), and polyvinylidene fluoride (PVDF) membrane filters (0.22 μm) at varying conjugate concentrations (1-10 mg/mL). At higher concentrations (10 mg/mL), the filtration process encountered challenges with some conjugates. For the preferred conjugates formulated at 10 mg/mL, the filtrates were analysed by both UV-Vis and NMR to confirm acceptable to good recovery rates. PES filters proved more effective than CA and PVDF filters in maintaining the structural integrity of the conjugate and achieving optimal filtration performance. The filtered conjugates were also assessed in vitro for release kinetics.

### Examples—In Vitro Biology

Release Kinetics of Ion Channel Modulator from Polymer

The concentration of free capsaicin released was measured using Single Cell Patch Clamp. Compounds synthesized in the previous examples were solubilised in various buffers and aqueous solutions, covering a range of pH. These preparations are referred to as compositions in the subsequent section. Samples were taken periodically over a 28-day period, and capsaicin concentrations were measured using Single Cell Patch Clamp to capture the release kinetics. The release of capsaicin was gradual and sustained throughout the experiment, with the concentration in the samples increasing progressively over time.

### In Vitro Assessment of Selected Exemplary Compounds-Efficacy in Single Cell Patch Clamp

### a) Materials and Methods

All chemical, cell culture media and reagents are purchased from Sigma-Aldrich (Ireland), Fisher Scientific, Lonza, Abcam, and Alomone labs unless otherwise stated.

### b) Cell Culture

CHO-hTRPV1 cells are cultured in DMEM medium supplemented with 10% foetal bovine serum (FBS), 2 mM glutamine, 1% Pen/strep and 1% non-essential amino acids. Cells are fed every two days and cultured at a density of 0.5 million/mL in a petri dish one day before the experiment. Exemplary compositions A, B D, E, and F (Table 1) were solubilised in phosphate buffer containing PEG 400 and mixed 1:1 v/v with synovial fluid at 37° C. with agitation (to model in vivo conditions). Exemplary compositions A and B were solubilised in phosphate buffer containing PEG 400 and were additionally tested at 4° C. Samples were taken periodically out for approximately 10 days and tested using a patch clamp.

### c) Single-Cell Patch Clamp

The efficacy of some exemplary compositions (Table 1) was tested using an automated patch-clamp system to measure and screen the effect of control materials and test compositions on the modulation of current in CHO-hTRPV1 cells. TRPV-1 activation elicits an initial increase in current flow into cells, followed by desensitization and reduction in current. The former is associated with an acute increase in pain in vivo, while the reduction in current translates into a reduction in pain in vivo. Single-cell patch-clamp recording on Patchliner was performed using an extracellular solution containing 140 mM NaCl, 4 mM KCL, 2 mM CaCl₂), 1 mM D-glucose monohydrate, 10 mM Hepes at pH 7.4 and osmolarity of 298 mOsm, and an intracellular solution containing 10 mM EGTA, 10 mM Hepes, 10 mM KCL, 10 mM NaCl, 110 mM KF at pH of 7.2 and osmolarity of over 280 mOsm. The Patchliner system is a fully automated, robust whole-cell patch clamp device, ideal for ion channel biophysics and mechanism of action and more sophisticated assays, including heat activation of TRPV1 channels. The recordings were made using an EPC10 patch clamp amplifier from HEKA. Whole-cell-induced currents were recorded in the voltage clamp mode in a holding potential of −60 mV at the baseline and in the presence of each test composition. Free capsaicin release from the compositions were calculated and quantified using the capsaicin standard curve.

TABLE 1
Compositions tested in the in vitro patch clamp study

| Composition | |
|---|---|
| code | Description |
| A | HA (MW: 500-1000 kDa) conjugated to Capsaicin |
| | derivative (CAP) at 11-15% w/w (example 10 above) |
| D | HA (MW: 500-1000 kDa) conjugated to Capsaicin |
| | derivative (CAP) at 6-10% w/w (example 12 above) |
| E | HA (MW: 500-1000 kDa) conjugated to Capsaicin |
| | derivative(CAP) at 11-15% w/w, (example 20 above) |
| F | HA (MW: 500-1000 kDa) conjugated to Capsaicin |
| | derivative(CAP) at 6-10% w/w, (example 14 above) |
| G | HA (MW: 500-1000 kDa) conjugated to Capsaicin |
| | -derivative (CAP) at 6-10% w/w, (example 16 above) |

### d) Results

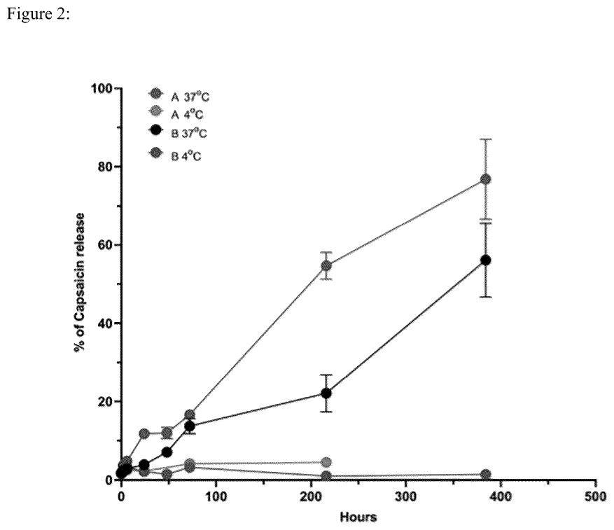

FIG. 2 illustrates the release kinetics of exemplary compositions A and B under different conditions (4° C. and 37° C.). Compositions were prepared at 1 mg/mL and the levels of free capsaicin assayed using patch clamp at various timepoints. The results show the compositions to have good stability at 4° C. with no significant capsaicin release over a >7-day time-course. Both Compositions A and B show release of capsaicin over 12.5% of the conjugated load by day 3 at 37° C. with agitation, and over 50% by day 16. Composition A shows faster release kinetics compared to Composition B. Neither composition showed a burst release. The in vitro kinetic release profile aligns with the expected in vivo behaviour, minimizing initial sensitization through an effective lower initial dose compared to a bolus administration. Pain relief is provided by the sustained release of the ion-channel modulator from the composition, maintaining therapeutic levels and extending the duration of action.

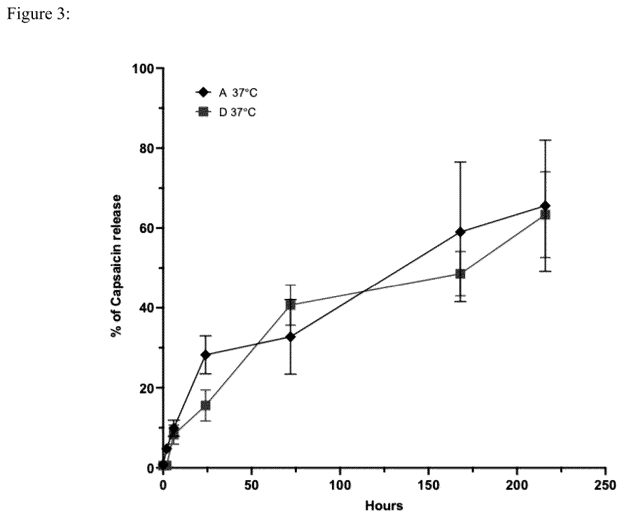

FIG. 3 illustrates the release kinetics of exemplary compositions A and D in modelled in vivo conditions. The levels of free capsaicin were assayed using patch clamp at various timepoints up to approximately 10 days. The results show release of capsaicin of over 50% of the conjugated load by day 10 at 37° C. Further, the release kinetics of both compositions are not significantly different.

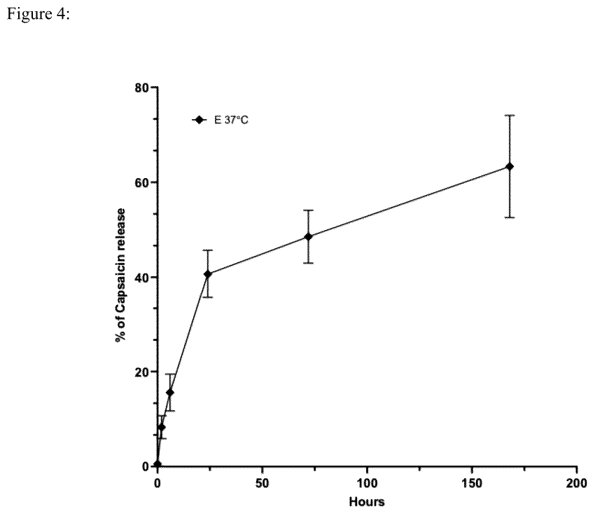

FIG. 4 illustrates the release kinetics of exemplary composition E in modelled in vivo conditions. The levels of free capsaicin were assayed using patch clamp at various timepoints up to approximately 10 days. The results show release of capsaicin of over 50% of the conjugated load by day 7 at 37° C. with agitation in synovial fluid. The results demonstrate favourable release kinetics under in vivo conditions (37° C. with agitation).

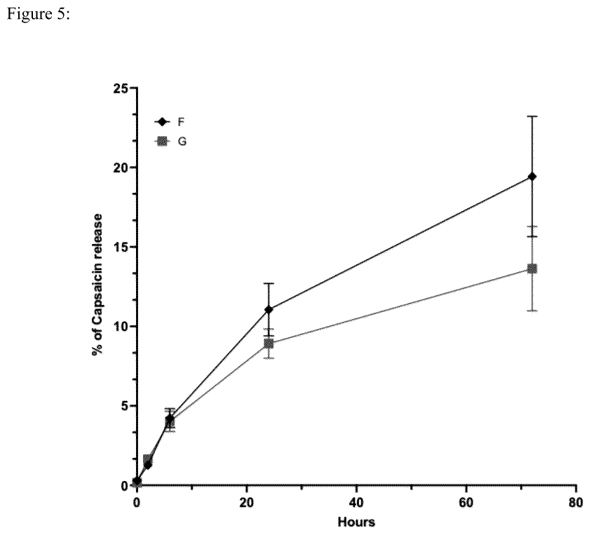

FIG. 5 illustrates the release kinetics of exemplary compositions F and G in modelled in vivo conditions. The levels of free capsaicin were assayed using patch clamp at various timepoints up to approximately 10 days. The results show slower capsaicin release under the same conditions of 37° C. with agitation in synovial fluid than those of Compositions A and D particularly (FIG. 2) over the first 100 hours. This shows the advantage of exploring several derivatives of capsaicin conjugated to HA in tuning and slowing down release of free capsaicin. The results demonstrate favourable release kinetics under in vivo conditions (37° C. with agitation).

### Examples—In Vivo Biology

In Vivo Assessment—näive Animals

### a) Objective

The objective was to investigate the safety of exemplary composition A (Table 1) at different doses in näive animals. Composition A has a TRPV1 agonist (capsaicin) as the ion channel modulator and the present invention minimised the initial excitation phase characteristic of these modulators. This demonstrates the superiority of the conjugation chemistry in lowering acute sensitisation. This initial excitation phase was most acute in the first hours after intra-articular administration of free TRPV1 agonist in rats.

### b) Materials and Methods

Details on the compositions, chemicals, induction agents, and treatments used in this study are summarised in Table 2.

TABLE 2
Treatments used in the in vivo study

| | Ion | | | |
|---|---|---|---|---|
| Treatment | Channel | | | |
| Name | Modulator | Solvent | Concentration | Notes |
| Vehicle | N/A | PBS | N/A | / |
| CAP | Free (E)- | PBS | 10 μg/50 μL | / |
| | Capsaicin | | | |
| HA | | PBS | 50 μg/50 μL | / |
| CAP + HA | Free (E)- | PBS | 1.25 μg/50 μL | Unconjugated |
| | Capsaicin | | | components of |
| | | | | Composition A |
| Composition | (E)- | PBS | 1.25 μg/50 μL | Tested at three |
| A | Capsaicin | | 6.25 μg/50 μL | strengths with |
| | | | 12.5 μg/50 μL | loading of 10 μg, |
| | | | | 50 μg, 100 μg |
| | | | | of capsaicin. |

### Treatment Administration

Under brief isoflurane anaesthesia, naïve rats (Sprague Dawley, female, 8 weeks old, n=4 per group, supplier: Charles River UK) received an intra-articular injection of the assigned treatment (50 μL) into the left knee using a 30 G needle-tipped insulin syringe.

### Evaluation of Pain-Related Behaviour

Pain Related Behaviours were Assessed Using:
Ethovision scored behavioural observation in the first 15 minutes after treatment-nocifensive behaviours including pain and immobility were measured in terms of duration (seconds) and frequency of response for each treatment.
Von Frey (VF) test-secondary hypersensitivity at the ipsilateral hind paw were measured using an electronic von Frey anaesthesiometer. Pressure was applied on the plantar surface until a response (withdrawal, flinching, licking) observed indicative of mechanical hypersensitivity. The measurement was performed once at baseline and up to 2 days following treatment administration.

### c) Results

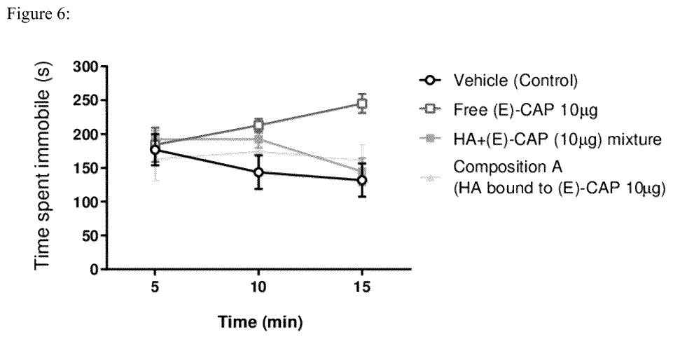

FIG. 6 shows the duration of immobility (nocifensive behaviour) displayed by the animals for the first 15 minutes following intra-articular treatment administration. The results show that the HA+ (E)-CAP mixture and exemplary composition A led to a duration of immobility (as an index of pain) equivalent to that of control (vehicle treatment), and less than that of free capsaicin. This result demonstrates the benefit of conjugating capsaicin in reducing the initial acute period of sensitisation.

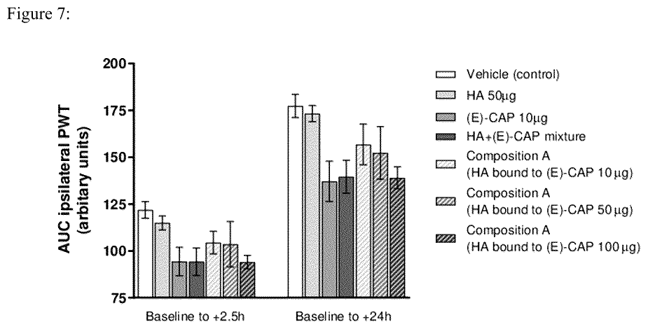

FIG. 7 shows the results for exemplary composition A in the VF test. Following administration of treatments, VF test was performed in naïve rats to measure paw withdrawal threshold (PWT) at the ipsilateral hind paw. The area under the curve (AUC) was calculated over time post-treatment with respect to baseline. The results are shown at +2.5 h (left side columns) and +24 h (right side columns) post-treatment. In each set of columns from left to right test materials are Vehicle, HA (sodium hyaluronate), CAP (free (E)-capsaicin), CAP+HA (free (E)-capsaicin+HA), and exemplary composition A (capsaicin derivative conjugated to HA) at three different concentration levels (×1, ×5, ×10 that of the CAP+HA concentration level). The results show that the exemplary composition A has a higher PWT compared to both free (E)-capsaicin (CAP) and the (E)-capsaicin+HA (CAP+HA) mixture groups, demonstrating the benefit of conjugation of the capsaicin derivative to HA in allowing loading of up to 10 times the levels of free (E)-capsaicin without the same level of acute sensitisation (i.e. pain) as capsaicin alone (CAP) or the mixture group (CAP+HA).

### In Vivo Assessment-MIA Model of Osteoarthritis

### a) Objective

The objective was to investigate the efficacy and safety of different compositions and at different doses in the rat sodium monoiodoacetate (MIA) model of knee osteoarthritis (OA). The MIA model is a standard preclinical model for mimicking joint disruption in OA and assessing of subsequent OA-related chronic pain behaviours.

### b) Materials and Methods

Details on the compositions, chemicals, induction agents, and treatments to be used in this study are summarised in Tables 3, 4, 5 and 6 respectively.

TABLE 3
Composition to be tested in the in vivo study

| Composition | |
|---|---|
| code | Composition |
| A | HA (MW: 500-1000 kDa) conjugated to Capsaicin |
| | derivative (CAP) at 11-15% w/w (example 10 above) |

TABLE 4
Chemicals to be used in the in vivo study.

| | Abbreviated | | | Batch |
|---|---|---|---|---|
| Chemical Name | Name | Constituents | Company | Number |
| Phosphate Buffered | PBS | Sodium chloride 9 mg; | Sigma | N/A |
| Saline | | Potassium Dihydrogen | Aldrich | |
| | | Phosphate 0.03 mg; | (Merck) | |
| | | Disodium Hydrogen | unless stated | |
| | | Phosphate Dihydrate (Merck) | | |
| | | 0.14 mg; Water for Injection | | |
| | | q.s. 1 ml | | |
| Sodium | MIA | Sodium iodoacetate ≥ 98% | Sigma | SLBZ7569 |
| monoiodoacetate | | | Aldrich | |
| | | | (Merck) | |

TABLE 5
Induction agents to be used in the in vivo study

| Induction Agent Name | Solute | Solvent | Concentration |
|---|---|---|---|
| MIA | MIA | PBS | 2 mg/50 μL |

TABLE 6
Treatments to be used in the in vivo study

| Treatment | Ion Channel | | | |
|---|---|---|---|---|
| Name | Modulator | Solvent | Concentration | Notes |
| Vehicle | | PBS/DMSO | N/A | |
| CAP | Free (E)- | PBS/DMSO | 0.2 mg/ml | Dose administered = 10 |
| | Capsaicin | (1:1) | | μg/50 μL |
| A | (E)-Capsaicin | PBS | 2.5, 5, and | Three different |
| (HA conjugated | | | 10 mg/ml | concentrations used - |
| to Capsaicin | | | | compare dose effects |
| derivative) | | | | |

### Animals

The study was conducted using Sprague-Dawley rats (8-10 weeks old, female, n=6-11 per group, supplier: Charles River UK). The animals were maintained under standard laboratory conditions and housed in groups of 2-3 rats per cage in standard plastic bottom cages containing woodchip bedding with nesting material and a plastic tube as enrichment. Food and water will be available ad libitum throughout the study.

### Induction of the MIA Model

To induce OA-like lesion in the knee joint, rats received a single intra-articular injection of MIA 2 mg (Table 5) into the left knee, using a sterile 30 G needle-tipped insulin syringe at a volume of 50 μL under brief anaesthesia (2-3% isoflurane, 0.8 Lmin⁻¹ O₂) on Day 0.

### Treatment Administration

On Day 18, rats were randomly allocated to treatment groups and 50 μL of the assigned treatment (Table 6) was administered under brief isoflurane anaesthesia into the left knee joint via intra-articular injection using a 30 G needle-tipped insulin syringe.

### Evaluation of Pain-Related Behaviour

Pain-Related Behaviours were Assessed Using:
Ethovision scored behavioural observation in the first 20 minutes after treatment administration-nocifensive behaviours including pain and immobility were measured in terms of duration (seconds) and frequency of response for each treatment.
Weight bearing (WB) test-primary hypersensitivity at the ipsilateral/injured knee joint (spontaneous knee joint pain at rest) was measured using an incapacitance tester (Linton Instrumentation, UK). The animal was introduced into the chamber and the pressure exerted (in grams) by each hind limb on the force plates was recorded over a period of 3 seconds. The weight distribution on the ipsilateral hind limb was calculated as a percentage of total weight exerted by both hind limbs. The measurement was performed once a day at baseline and up to 42 days (6 weeks) post-treatment (up to 60 days post-MIA).

### c) Results

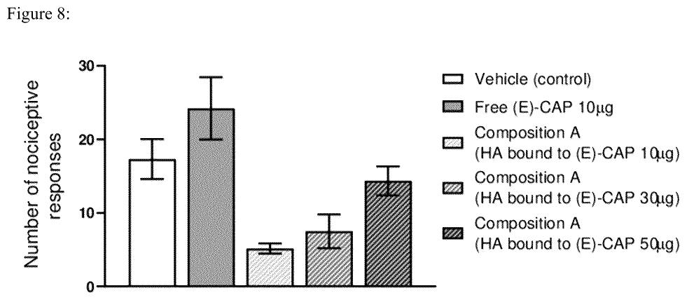

FIG. 8 illustrates the results for number of nociceptive responses (flinching, licking, shaking) displayed by the MIA rats for the first 20 minutes following intra-articular treatment administration. The results show that the exemplary composition A (Table 3) at three different concentration levels (×1, ×3, and ×5 that of free (E)-CAP concentration level) led to reduced pain-related responses that is equivalent to that of control (vehicle treatment) and less than that of free (E)-capsaicin. This result demonstrates the benefit of conjugating capsaicin derivative to HA in lowering the initial acute period of sensitisation (i.e. pain) in the MIA model of knee OA pain.

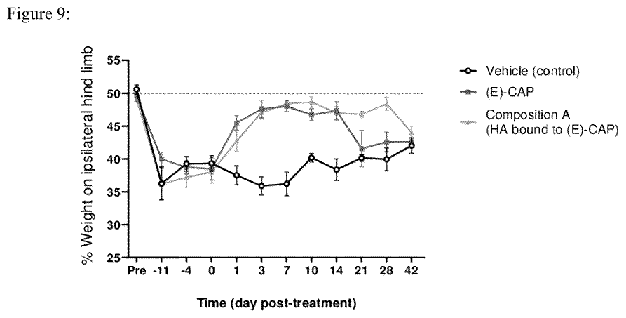

FIG. 9 shows the results for exemplary composition A (Table 1) in the WB test. Following MIA injection, the weight bearing on the ipsilateral (injured) hind limb was reduced indicating weight bearing asymmetry as a measure of spontaneous joint pain. The results show that following intra-articular treatment administration, the exemplary composition A improved this weight bearing deficit in the MIA rats, indicating alleviation of knee OA-related joint pain, for up to 4 weeks compared to free (E)-Capsaicin (CAP) with effect up to 2 weeks post-treatment. This result demonstrates the benefit of conjugating capsaicin derivative to HA in achieving a prolonged, sustained pain-relieving effect that persists longer than capsaicin alone (CAP).

1. A composition for treatment of pain, wherein the composition comprises a TRPV1 ion channel modulator derivative, capable of covalently linking to a polymer, wherein the ion channel modulator derivative is covalently linked to a polymer, and alternatively or additionally the ion channel modulator derivative is mixed with the polymer.

2. The composition of claim 1, wherein the TRPV1 channel modulator derivative is a capsaicinoid.

3. The composition of claim 2, wherein the capsaicinoid is characterized by a modification to allow covalent linking of the capsaicinoid to a polymer via ester bonds, amide bonds, carbamate bonds, carbonate bonds, or cleavable ether bonds with building blocks selected from:
aliphatic or aromatic dicarboxylic acids preferably succinic acid, adipic acid, or glutaric acid;
hydroxy acids preferably lactic acid, glycolic acid, or citric acid;
amino-acids peptides preferably beta-alanine, glycine, lysine, or glutamic acid;
oligopeptides or polypeptides;
diamines preferably 1,6-diaminohexane or ethylene diamine;
aminoalcohols preferably serinol or 1-amino-2-propanol;
diols preferably ethylene glycol, 1,6-hexanediol, or glycerol;
alkyloxycarbonyloxymethyl; and/or
N-alkyl-N-alkyloxycarbonylaminomethyl.

4. The composition of claim 3, wherein the TRPV1 ion channel modulator derivative is selected from the group consisting of:
(E)-2-methoxy-4-[(8-methylnon-6-enamido)methyl]phenyl glycinate;
(E)-2-methoxy-4-[(8-methylnon-6-enamido)methyl]phenyl 3-aminopropanoate;
(E)-2-methoxy-4-[(8-methylnon-6-enamido)methyl]phenyl 2-[2-(2 aminoethoxy)ethoxy]acetate;
(E)-2-methoxy-4-[(8-methylnon-6-enamido)methyl]phenyl 3-(2-aminoethoxy)propanoate;
(E)-2-methoxy-4-[(8-methylnon-6-enamido)methyl]phenyl 2-(2-aminoethoxy)acetate;
(E)-2-methoxy-4-[(8-methylnon-6-enamido)methyl]phenyl (5-aminopentyl)carbamate;
(E)-2-methoxy-4-[(8-methylnon-6-enamido)methyl]phenyl [2-(2-aminoethoxy)ethyl]carbamate;
2-methoxy-4-{[(E)-8-methylnon-6-enamido]methyl]phenyl trans-4-aminocyclohexane-1-carboxylate;
(E)-2-methoxy-4-[(8-methylnon-6-enamido)methyl]phenyl 3-aminopyrrolidine-1-carboxylate;
(E)-2-methoxy-4-[(8-methylnon-6-enamido)methyl]phenyl (2-aminoethyl)(isopropyl)carbamate;
2-methoxy-4-{[(E)-8-methylnon-6-enamido]methyl}phenyl L-isoleucinate;
2-methoxy-4-{[(E)-8-methylnon-6-enamido]methyl}phenyl trans-4-(2-aminoacetamido)cyclohexane-1-carboxylate;
2-methoxy-4-{[(E)-8-methylnon-6-enamido]methyl}phenyl trans-4-(2-aminopropanamido)cyclohexane-1-carboxylate;
(E)-2-methoxy-4-[(8-methylnon-6-enamido)methyl]phenyl (3-aminopropanoyl)-L-prolinate;
(E)-2-methoxy-4-[(8-methylnon-6-enamido)methyl]phenyl piperidine-2-carboxylate;
(E)-2-methoxy-4-[(8-methylnon-6-enamido)methyl]phenyl glycyl-L-prolinate;
(E)-2-methoxy-4-[(8-methylnon-6-enamido)methyl]phenyl 1-glycylpiperidine-2-carboxylate;
(E)-2-methoxy-4-[(8-methylnon-6-enamido)methyl]phenyl 1-(3-aminopropanoyl) piperidine-2-carboxylate;
(E)-2-methoxy-4-[(8-methylnon-6-enamido)methyl]phenyl 2-[2-(2-aminoethoxy)ethoxy]acetate;
(E)-2-methoxy-4-[(8-methylnon-6-enamido)methyl]phenyl L-valinate;
(E)-2-methoxy-4-[(8-methylnon-6-enamido)methyl]phenyl (3-aminopropanoyl)-L-valinate;
2-methoxy-4-{[(E)-8-methylnon-6-enamido]methyl}phenyl (1S,2R)-2-aminocyclopentane-1-carboxylate;
(E)-2-methoxy-4-[(8-methylnon-6-enamido)methyl]phenyl (3-aminopropanoyl)-D-prolinate;
(E)-2-methoxy-4-[(8-methylnon-6-enamido)methyl]phenyl (3-aminopropanoyl)-DL-prolinate; and
their salts or free forms thereof.

5. The composition of claim 1, wherein the composition comprises one or more of:
E)-2-methoxy-4-[(8-methylnon-6-enamido)methyl]phenyl glycinate-introduced sodium hyaluronate;
(E)-2-methoxy-4-[(8-methylnon-6-enamido)methyl]phenyl 3-aminopropanoate-introduced sodium hyaluronate;
(E)-2-methoxy-4-[(8-methylnon-6-enamido)methyl]phenyl 3-aminopropanoate-introduced sodium hyaluronate;
(E)-2-methoxy-4-[(8-methylnon-6-enamido)methyl]phenyl 3-(2-aminoethoxy)propanoate-introduced sodium hyaluronate;
(E)-2-methoxy-4-[(8-methylnon-6-enamido)methyl]phenyl 3-aminopropanoate-introduced sodium hyaluronate;
(E)-2-methoxy-4-[(8-methylnon-6-enamido)methyl]phenyl 3-aminopropanoate-introduced sodium hyaluronate;
(E)-2-methoxy-4-[(8-methylnon-6-enamido)methyl]phenyl 3-(2-aminoethoxy)propanoate-introduced sodium hyaluronate;
(E)-2-methoxy-4-[(8-methylnon-6-enamido)methyl]phenyl 3-aminopropanoate-introduced sodium hyaluronate;
(E)-2-methoxy-4-[(8-methylnon-6-enamido)methyl]phenyl 3-(2-aminoethoxy)propanoate-introduced sodium hyaluronate;
(E)-2-methoxy-4-[(8-methylnon-6-enamido)methyl]phenyl 3-(2-aminoethoxy)propanoate-introduced sodium hyaluronate;
(E)-2-methoxy-4-[(8-methylnon-6-enamido)methyl]phenyl 3-(2-aminoethoxy)propanoate-introduced sodium hyaluronate;
(E)-2-methoxy-4-[(8-methylnon-6-enamido)methyl]phenyl [2-(2-aminoethoxy)ethyl]carbamate-introduced sodium hyaluronate;
(E)-2-methoxy-4-[(8-methylnon-6-enamido)methyl]phenyl (5-aminopentyl)carbamate-introduced sodium hyaluronate;
(E)-2-methoxy-4-[(8-methylnon-6-enamido)methyl]phenyl 3-aminopyrrolidine-1-carboxylate-introduced sodium hyaluronate;
2-methoxy-4-{[(E)-8-methylnon-6-enamido]methyl]phenyl trans-4-aminocyclohexane-1-carboxylate-introduced sodium hyaluronate;
2-methoxy-4-{[(E)-8-methylnon-6-enamido]methyl}phenyl L-isoleucinate-introduced sodium hyaluronate;
(E)-2-methoxy-4-[(8-methylnon-6-enamido)methyl]phenyl (2-aminoethyl)(isopropyl)carbamate-introduced sodium hyaluronate;
2-methoxy-4-{[(E)-8-methylnon-6-enamido]methyl}phenyl trans-4-(2-aminoacetamido)cyclohexane-1-carboxylate-introduced sodium hyaluronate;
2-methoxy-4-{[(E)-8-methylnon-6-enamido]methyl}phenyl trans-4-(2-aminopropanamido)cyclohexane-1-carboxylate-introduced sodium hyaluronate;
(E)-2-methoxy-4-[(8-methylnon-6-enamido)methyl]phenyl piperidine-2-carboxylate-introduced sodium hyaluronate;
(E)-2-methoxy-4-[(8-methylnon-6-enamido)methyl]phenyl (3-aminopropanoyl)-L-prolinate-introduced sodium hyaluronate;
(E)-2-methoxy-4-[(8-methylnon-6-enamido)methyl]phenyl glycyl-L-prolinate-introduced sodium hyaluronate;
(E)-2-methoxy-4-[(8-methylnon-6-enamido)methyl]phenyl 1-glycylpiperidine-2-carboxylate-introduced sodium hyaluronate;
(E)-2-methoxy-4-[(8-methylnon-6-enamido)methyl]phenyl 1-(3 aminopropanoyl) piperidine-2-carboxylate-introduced sodium hyaluronate;
(E)-2-methoxy-4-[(8-methylnon-6-enamido)methyl]phenyl 2-[2-(2-aminoethoxy)ethoxy]acetate-introduced sodium hyaluronate;
(E)-2-methoxy-4-[(8-methylnon-6-enamido)methyl]phenyl glycinate-introduced sodium hyaluronate;
(E)-2-methoxy-4-[(8-methylnon-6-enamido)methyl]phenyl L-valinate-introduced sodium hyaluronate;
(E)-2-methoxy-4-[(8-methylnon-6-enamido)methyl]phenyl (3-aminopropanoyl)-L-valinate-introduced sodium hyaluronate; and
2-methoxy-4-{[(E)-8-methylnon-6-enamido]methyl}phenyl (1S,2R)-2-aminocyclopentane-1-carboxylate-introduced sodium hyaluronate.

6. The composition of claim 1, wherein the polymer is selected from the group consisting of hyaluronic acid (HA), derivatives of hyaluronic acid, sodium hyaluronate, derivatives of sodium hyaluronate, polysaccharides, and any combination thereof.

7. The composition of claim 1, wherein the mass:mass ratio of polymer to covalently bound ion channel modulator is in a range 1:0.001 to 1:0.5, preferably in range 1:0.01 to 1:0.3.

8. The composition of claim 7, wherein the polymer is hyaluronic acid or sodium hyaluronate having a molecular weight between 500 and 3000 kDa.

9. The composition of claim 1, wherein a TRPV1 ion channel modulator is released from the TRPV1 ion channel modulator derivative within 0 to 12 months.

10. The composition of claim 1, wherein the composition is suitable for administration as an injection.

11. The composition of claim 1, wherein the composition is in a phosphate buffered solution with an osmolality physiologically compatible and that is heat or filtered sterilized.

12. A method to release a TRPV1 ion channel modulator from a TRPV1 ion channel modulator derivative contained in the composition of claim 1 via hydrolysis in a sustained and prolonged manner at a location of pain and/or in vicinity of the pain, said method comprising injecting the composition of any of the previous claims at a location of pain and/or in vicinity of the pain.

13. The method of claim 12, wherein an injection volume is 1-10 mL and the injection volume comprises from 1 μg to 100 mg of ion channel modulator derivative.

14. The method of claim 12 for the treatment of chronic pain conditions.

15. The method of claim 12, wherein the pain is joint pain.

16. The method of claim 12, wherein the pain is osteoarthritic joint pain.

17. The composition of claim 1 for use as an injectable composition for treating chronic pain conditions, including joint pain and osteoarthritic joint pain.

18. The method of claim 13 for the treatment of chronic pain conditions.

19. The method of claim 13, wherein the pain is joint pain.

20. The method of claim 13, wherein the pain is osteoarthritic joint pain.
